SEQUENCE LISTING (62 sequences)

SEQ ID NO: 1 - protein, 206 aa, Artificial Sequence
  Synthetic Peptide
EGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 2 - protein, 155 aa, Artificial Sequence
  Synthetic Peptide
TKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 3 - protein, 155 aa, Artificial Sequence
  Synthetic Peptide
TKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 4 - protein, 208 aa, Artificial Sequence
  Synthetic Peptide
STINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 5 - protein, 113 aa, Artificial Sequence
  Synthetic Peptide
PIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 6 - protein, 156 aa, Artificial Sequence
  Synthetic Peptide
NQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 7 - protein, 203 aa, Artificial Sequence
  Synthetic Peptide
MEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 8 - protein, 156 aa, Artificial Sequence
  Synthetic Peptide
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 9 - protein, 176 aa, Artificial Sequence
  Synthetic Peptide
FTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 10 - protein, 156 aa, Artificial Sequence
  Synthetic Peptide
NQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 11 - protein, 200 aa, Artificial Sequence
  Synthetic Peptide
GHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 12 - protein, 236 aa, Artificial Sequence
  Synthetic Peptide
YEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 13 - protein, 137 aa, Artificial Sequence
  Synthetic Peptide
GMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 14 - protein, 153 aa, Artificial Sequence
  Synthetic Peptide
KYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 15 - protein, 163 aa, Artificial Sequence
  Synthetic Peptide
AVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 16 - protein, 174 aa, Artificial Sequence
  Synthetic Peptide
GANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 17 - protein, 207 aa, Artificial Sequence
  Synthetic Peptide
TDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 18 - protein, 127 aa, Artificial Sequence
  Synthetic Peptide
ITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 19 - protein, 234 aa, Artificial Sequence
  Synthetic Peptide
GSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 20 - protein, 161 aa, Artificial Sequence
  Synthetic Peptide
SGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 21 - protein, 156 aa, Artificial Sequence
  Synthetic Peptide
GDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 22 - protein, 156 aa, Artificial Sequence
  Synthetic Peptide
ILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 23 - protein, 155 aa, Artificial Sequence
  Synthetic Peptide
TKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 24 - protein, 208 aa, Artificial Sequence
  Synthetic Peptide
DDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 25 - protein, 113 aa, Artificial Sequence
  Synthetic Peptide
PIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 26 - protein, 113 aa, Artificial Sequence
  Synthetic Peptide
PIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 27 - protein, 156 aa, Artificial Sequence
  Synthetic Peptide
NQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 28 - protein, 156 aa, Artificial Sequence
  Synthetic Peptide
NQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 29 - protein, 202 aa, Artificial Sequence
  Synthetic Peptide
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 30 - protein, 202 aa, Artificial Sequence
  Synthetic Peptide
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 31 - protein, 202 aa, Artificial Sequence
  Synthetic Peptide
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 32 - protein, 156 aa, Artificial Sequence
  Synthetic Peptide
NQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 33 - protein, 156 aa, Artificial Sequence
  Synthetic Peptide
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 34 - protein, 156 aa, Artificial Sequence
  Synthetic Peptide
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 35 - protein, 155 aa, Artificial Sequence
  Synthetic Peptide
  VARIANT (69)..(69): Xaa is A or K
TKKVGIVDTTFARVDMASAAILTLKMESPNIKIIRKTVPGIKDLPVACKKLLEEEGCDIVMALGMPGKXEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 208 aa, Artificial Sequence
  Synthetic Peptide
  VARIANT (1)..(1): Xaa is S or D
  VARIANT (2)..(2): Xaa is T or D
  VARIANT (9)..(9): Xaa is A or R
  VARIANT (84)..(84): Xaa is T or D
  VARIANT (118)..(118): Xaa is A or Q
  VARIANT (162)..(162): Xaa is S or D
  VARIANT (188)..(188): Xaa is T or R
XXINNQLKXLKVIPVIAIDNAEDIIPLGKVLAENGLPAAEITFRSSAAVKAIMLLRSAQPEMLIGAGTILNGVQALAAKEAGAXFVVSPGFNPNTVRACQIIGIDIVPGVNNPSTVEXALEMGLTTLKFFPAEASGGISMVKSLVGPYGDIRLMPTGGITPXNIDNYLAIPQVLACGGTWMVDKKLVXNGEWDEIARLTREIVEQVNP

SEQ ID NO: 37 - protein, 113 aa, Artificial Sequence
  Synthetic Peptide
  VARIANT (12)..(12): Xaa is T or D
  VARIANT (32)..(32): Xaa is K or E
  VARIANT (70)..(70): Xaa is S or D
  VARIANT (73)..(73): Xaa is R or E
  VARIANT (100)..(100): Xaa is N or D
  VARIANT (102)..(102): Xaa is N or D
PIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 38 - protein, 156 aa, Artificial Sequence
  Synthetic Peptide
  VARIANT (8)..(8): Xaa is Y or H
  VARIANT (81)..(81): Xaa is N or D
  VARIANT (86)..(86): Xaa is R or D
  VARIANT (104)..(104): Xaa is S or D
  VARIANT (118)..(118): Xaa is R or E
  VARIANT (120)..(120): Xaa is R or E
  VARIANT (123)..(123): Xaa is A or D
  VARIANT (125)..(125): Xaa is H or D
  VARIANT (127)..(127): Xaa is R or E
  VARIANT (149)..(149): Xaa is A or N
NQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 39 - protein, 202 aa, Artificial Sequence
  Synthetic Peptide
  VARIANT (123)..(123): Xaa is T or D
  VARIANT (136)..(136): Xaa is Q or E
  VARIANT (139)..(139): Xaa is K or E
  VARIANT (157)..(157): Xaa is N or D
  VARIANT (160)..(160): Xaa is N or D
  VARIANT (163)..(163): Xaa is E or K
  VARIANT (166)..(166): Xaa is D or K
  VARIANT (176)..(176): Xaa is S, D or K
  VARIANT (180)..(180): Xaa is K or E
  VARIANT (182)..(182): Xaa is T, D, or K
  VARIANT (189)..(189): Xaa is D or K
  VARIANT (192)..(192): Xaa is A, E, or K
  VARIANT (195)..(195): Xaa is E or K
  VARIANT (196)..(196): Xaa is E or K
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 40 - protein, 156 aa, Artificial Sequence
  Synthetic Peptide
  VARIANT (8)..(8): Xaa is Y or H
  VARIANT (37)..(37): Xaa is A or R
  VARIANT (81)..(81): Xaa is N or D
  VARIANT (86)..(86): Xaa is R or D
  VARIANT (96)..(96): Xaa is N or D
  VARIANT (104)..(104): Xaa is S, N, or D
  VARIANT (118)..(118): Xaa is R, E, or N
  VARIANT (120)..(120): Xaa is R, E, or D
  VARIANT (121)..(121): Xaa is K or D
  VARIANT (123)..(123): Xaa is K or D
  VARIANT (125)..(125): Xaa is H or D
NQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 41 - protein, 158 aa, Artificial Sequence
  Synthetic Peptide
GEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 42 - protein, 183 aa, Artificial Sequence
  Synthetic Peptide
SQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 43 - protein, 102 aa, Artificial Sequence
  Synthetic Peptide
EEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 44 - protein, 121 aa, Artificial Sequence
  Synthetic Peptide
VRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 45 - protein, 176 aa, Artificial Sequence
  Synthetic Peptide
SKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 46 - protein, 121 aa, Artificial Sequence
  Synthetic Peptide
VRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 47 - protein, 171 aa, Artificial Sequence
  Synthetic Peptide
RITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 48 - protein, 122 aa, Artificial Sequence
  Synthetic Peptide
PHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 49 - protein, 157 aa, Artificial Sequence
  Synthetic Peptide
ESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 50 - protein, 112 aa, Artificial Sequence
  Synthetic Peptide
PVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 51 - protein, 102 aa, Artificial Sequence
  Synthetic Peptide
EEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 52 - protein, 119 aa, Artificial Sequence
  Synthetic Peptide
NLAEKMYKAGNAMYRKGQYTIAIIAYTLALLKDPNNAEAWYNLGNAAYKKGEYDEAIEAYQKALELDPNNAEAWYNLGNAYYKQGDYDEAIEYYKKALRLDPRNVDAIENLIEAEEKQG

SEQ ID NO: 53 - protein, 119 aa, Artificial Sequence
  Synthetic peptide
EEAELAYLLGELAYKLGEYRIAIRAYRIALKRDPNNAEAWYNLGNAYYKQGDYREAIRYYLRALKLDPENAEAWYNLGNALYKQGKYDLAIIAYQAALEEDPNNAEAKQNLGNAKQKQG

SEQ ID NO: 54 - protein, 119 aa, Artificial Sequence
  Synthetic Peptide
NSAEAMYKMGNAAYKQGDYILAIIAYLLALEKDPNNAEAWYNLGNAAYKQGDYDEAIEYYQKALELDPNNAEAWYNLGNAYYKQGDYDEAIEYYEKALELDPNNAEALKNLLEAIAEQD

SEQ ID NO: 55 - protein, 187 aa, Artificial Sequence
  Synthetic Peptide
TDPLAVILYIAILKAEKSIARAKAAEALGKIGDERAVEPLIKALKDEDALVRAAAADALGQIGDERAVEPLIKALKDEEGLVRASAAIALGQIGDERAVQPLIKALTDERDLVRVAAAVALGRIGDEKAVRPLIIVLKDEEGEVREAAAIALGSIGGERVRAAMEKLAERGTGFARKVAVNYLETHK

SEQ ID NO: 56 - protein, 119 aa, Artificial Sequence
  Synthetic Peptide
EEAELAYLLGELAYKLGEYRIAIRAYRIALKRDPNNAEAWYNLGNAYYKQGDYDEAIEYYQKALELDPNNAEAWYNLGNAYYKQGDYDEAIEYYEKALELDPENLEALQNLLNAMDKQG

SEQ ID NO: 57 - protein, 279 aa, Artificial Sequence
  Synthetic Peptide
IEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEKFMARAIMAIAILAAKAIEAIYRLADNHTSPTYIEKAIEAIEKIARKAIKAIEMLAKNITTEEYKEKAKKIIDIIRKLAKMAIKKLEDNRT

SEQ ID NO: 58 - protein, 153 aa, Artificial Sequence
  Synthetic Peptide
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 59 - protein, 119 aa, Artificial Sequence
  Synthetic Peptide
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 60 - protein, 157 aa, Artificial Sequence
  Synthetic Peptide
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 61 - protein, 205 aa, Artificial Sequence
  Synthetic Peptide
MEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 62 - protein, 12 aa, Artificial Sequence
  Synthetic Peptide
EKAAKAEEAARK

(19) United States
(12) Patent Application Publication
(10) Pub. No.: US 2024/0050559 A1
(43) Pub. Date: Feb. 15, 2024

(54) METHOD OF MAKING VIRUS-LIKE PARTICLE

(71) Applicant: Icosavax, Inc., Seattle, WA (US)

(72) Inventors: Charles RICHARDSON, Seattle, WA (US); Hans R. LIEN, Seattle, WA (US)

(21) Appl. No.: 18/000,546

(22) PCT Filed: Jun. 9, 2021
(86) PCT No.: PCT/US2021/036688

§ 371 Date: Dec. 2, 2022

Related U.S. Application Data
(60) Provisional application No. 63/036,535, filed on Jun. 9, 2020.

(51) Int. Cl. A61K 39/385; C12N 7/00

(52) U.S. Cl. CPC A61K 39/385; C12N 7/00; A61K 2039/5258

(57) ABSTRACT

Disclosed is a method of making a nanostructure by solubilizing a recombinant component B (compB) protein from inclusion bodies with a solubilization solution, thereby generating a product sample comprising product compB protein.

### RELATED APPLICATIONS

This application is a national stage entry of International Patent Application No. PCT/US2021/036688, filed Jun. 9, 2021, which claims priority to U.S. provisional application No. 63/036,535, filed Jun. 9, 2020, each of which is incorporated herein by reference in its entirety for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ICVX_008_01IWO_ST25.txt. The text file is 94 KB, created on Jun. 9, 2021, and is being submitted electronically via EFS-Web.

### FIELD OF INVENTION

The present disclosure relates generally to self-assembling protein nanostructures, in particular methods of making nanostructures, including nanostructure-based vaccines.

### BACKGROUND

Protein-based Virus-Like Particles (pbVLPs) provide a useful platform to present proteins or other macromolecules symmetrically. They can be distinguished from conventional VLPs made from viral capsid proteins (e.g., from a non-enveloped virus) or lipid-embedded proteins (e.g., extracted from an enveloped virus or made using recombinant membrane proteins mixed with lipids). The later do not generally have defined symmetry. The former are generally limited in their ability to display proteins, due to challenges in attaching proteins to viral capsids.

One application for VLPs generally and for pbVLPs in particular is as vaccines. Studies have demonstrated experimentally that antigens displayed on pbVLPs elicit stronger antibody responses than conventional subunit vaccines and non-symmetric VLPs.

Bale et al., Science 353:389-394 (2016) discloses various two-component icosahedral pbVLPs, including a set of pbVLPs made from protein components designated component A (compA) and component B (compB).

There remains a need in the art for methods of expressing, purifying, and assembling protein-based Virus-Like Particles. The present disclosure fulfills this need.

### SUMMARY OF THE INVENTION

After extensive experimentation, the present inventors have surprisingly discovered that component B (compB) proteins for two-component self-assembling protein-based Virus-Like Particles (pbVLPs) can be expressed and purified from inclusion bodies in as great, or greater, yield, purity, and/or biological activity as they can be from the soluble fraction of the recombinant expression system. Moreover, the purification procedures disclosed herein surprisingly do not require denaturing or refolding steps after solubilization of the compB protein from the inclusion bodies

Provided herein is a method of making a nanostructure, comprising solubilizing a recombinant component B (compB) protein from inclusion bodies with a solubilization solution, thereby generating a product sample comprising product compB protein.

In some embodiments, the solubilization solution comprises urea. The urea may be at a urea concentration of 0.15 M to 2 M, such as 0.5 M.

In some embodiments, the solubilization solution is a buffered solution having a pH of 7-8, optionally a pH of 7.4

In some embodiments, the solubilization solution comprises a zwitterionic surfactant.

In some embodiments, the zwitterionic surfactant is selected from CHAPSO (3-(3-Cholamidopropyl)dimethylammonio)-2-hydroxy-1-propanesulfonate), LDAO, DDMAB, and any Zwittergent® surfactant.

In some embodiments, the solubilization solution comprises 3-[(3-cholamidopropyl)dimethyl ammonio]-1-propanesulfonate (CHAPS).

In some embodiments, the method comprises, prior to the solubilization step, washing the inclusion bodies with a wash solution comprising urea, optionally at a urea concentration of less than 150 mM, optionally 50-150 mM.

In some embodiments, the method comprises contacting the compB protein with an anion exchange resin, optionally a weak anion exchange resin, optionally a diethylaminoethyl(DEAE)-conjugated resin; and eluting the compB protein from the resin using an elution solution.

In some embodiments, the method comprises, before the eluting step, washing the anion exchange resin with a column-wash solution, the column-wash solution comprising:
a zwitterionic surfactant, optionally 3-[(3-cholamidopropyl)dimethylammonio]-1-propanesulfonate (CHAPS) or an equivalent thereof, and/or
a nonionic surfactant, optionally Triton X-100 or an equivalent thereof

In some embodiments, the elution solution comprises sodium chloride (NaCl) at a NaCl concentration of 400 mM to 600 mM.

In some embodiments, the method comprises purifying the compB protein with a mixed-mode resin, optionally a ceramic hydroxyapatite (CHT) resin.

In some embodiments, the inclusion bodies are generated in a bacterial cell comprising a polynucleotide encoding the compB protein, the polynucleotide operatively linked to a promoter.

In some embodiments, the bacterial cell is cultured at less than about 33° C., optionally at about 15° C. to about 33° C. or at about 17° C. to about 30 ° C., preferably at about 30° C.

In some embodiments, the bacterial cell is an E. coli cell.

In some embodiments, the bacterial cell is a B-strain E. coli cell.

In some embodiments, the bacterial cell is a K12-strain E. coli cell.

In some embodiments, the promoter is a PhoA promoter.

In some embodiments, the promoter is a promoter other than a T7 promoter.

In some embodiments, the method comprises lysing the bacterial cell in a lysis solution, wherein the lysis solution is substantially free of agents that promote solubility of inclusion bodies; and recovering the inclusion bodies.

In some embodiments, the lysis solution is substantially free of detergents.

In some embodiments, the product compB protein has at least 50% solubility, optionally 70-95% solubility.

In some embodiments, solubility is measured by gel filtration chromatography, optionally using a Superose 6 column.

In some embodiments, the product compB protein has at least 80% purity calculated as weight by weight of total protein (w/w), optionally at least 95% w/w purity.

In some embodiments, purity is measured by poly-acrylamide gel electrophoresis, optionally denaturing SDS-PAGE.

In some embodiments, the product compB protein is at least 70% w/w assembly competent, optionally 90-98% w/w assembly competent.

In some embodiments, percentage of assembly competent compB protein is defined as the percentage of compB protein in the product solution, weight by weight (w/w), that assembles into a protein-based Virus-Like Particle (vpVLP) when the compB protein is mixed with a solution comprising component A (compA) protein in excess.

In some embodiments, the product solution comprises less than 50 endotoxin units per milligram of total protein (EU/mg), optionally 5-15 units of EU/mg.

In some embodiments, the method does not comprise denaturing the compB protein and/or does not comprise refolding the compB protein. In some embodiments, the method does not comprise denaturing the compB protein. In some embodiments, the method does not comprise refolding the compB protein. In some embodiments, the method involves generating a multimeric assembly without assembling the multimer from monomeric proteins. In some embodiments, the method involves generating pentameric assemblies without assembling pentamers from monomeric proteins.

In some embodiments, the yield of compB protein is between about 170-190 g/L wet cell weight (WCW) at harvest and/or about 1 g/L WCW compB protein in the inclusion bodies.

In some embodiments, the compB protein is a I53-50B protein.

In some embodiments, the I53-50B protein shares at least 95% identity to I53-50B.1 (SEQ ID NO:32), I53-50B.1NegT2 (SEQ ID NO:33), or I53-50B.4PosT1 (SEQ ID NO: 34).

In some embodiments, the I53-50B is any one of the proteins represented by SEQ ID NO: 40 (I53-50B genus).

In some embodiments, the I53-50B protein shares at least 99% identity to I53-50B.1 (SEQ ID NO:32), I53-50B.1NegT2 (SEQ ID NO:33), or I53-50B.4PosT1 (SEQ ID NO: 34).

In some embodiments, the I53-50B protein shares 100% identity to I53-50B.1 (SEQ ID NO:32), I53-50B.1NegT2 (SEQ ID NO:33), or I53-50B.4PosT1 (SEQ ID NO: 34).

In some embodiments, the compB protein is a I53_dn5A protein.

In some embodiments, the I53_dn5A protein shares at least 95% identity to SEQ ID NO: 169.

In some embodiments, the I53_dn5A protein shares at least 99% identity to SEQ ID NO: 169.

In some embodiments, the I53_dn5A protein shares at least 100% identity to SEQ ID NO: 169.

Further provided herein is a composition comprising compB protein produced by any of the methods described herein.

Further provided herein is composition comprising compB protein, wherein the compB protein is:
a. at least 50% soluble, optionally 70-95% soluble;
b. at least 80% pure, wherein purity is calculated as weight by weight of total protein (w/w), optionally at least 95% w/w pure; and/or
c. at least 70% w/w assembly competent, optionally 90-100% w/w assembly competent.

In some embodiments, the composition comprises one or more of 20 mM tris(hydroxymethyl)aminomethane (Tris) buffer, optionally at 20 mM, and/or 250 mM NaCl, optionally at 250 mM.

In some embodiments, the composition is buffered at a pH of 7-8, optionally a pH of 7.4.

In some embodiments, the composition is stable to storage and/or freeze-thaw.

In some embodiments, the composition is stable to storage and/or freeze-thaw.

In some embodiments, the disclosure provides methods of making a nanostructure, wherein the nanostructure comprises a component A (compA) protein and a component B (compB) protein, wherein the compB protein is solubilized from inclusion bodies with a solubilization solution, thereby generating isolated compB protein, wherein compA and the isolated compB form a nanostructure.

In some embodiments, a nanostructure is made by the methods described herein.

In some embodiments, compB of the nanostructure is encoded by a polypeptide sequence that has at least 90%, at least 95%, at least 99%, or 100% identity to any one of SEQ ID NOs: 2, 4, 6, 8, 10, 12, 14, 16, 18, 20, 22, 24, 27, 28, 32-34, 36, 38, 40, 42, 44, 46, 48, 50, 52, 54, 56, and 58.

In some embodiments, compA and the compB each comprise a polypeptide sequence that has at least 90%, at least 95%, at least 99%, or 100% identity to any one of
(i) SEQ ID NO:1 and SEQ ID NO:2 respectively;
(ii) SEQ ID NO:3 and SEQ ID NO:4 respectively;
(iii) SEQ ID NO:3 and SEQ ID NO:24 respectively;
(iv) SEQ ID NO:23 and SEQ ID NO:4 respectively;
(v) SEQ ID NO:35 and SEQ ID NO:36 respectively;
(vi) SEQ ID NO:5 and SEQ ID NO:6 respectively;
(vii) SEQ ID NO:5 and SEQ ID NO:27 respectively;
(viii) SEQ ID NO:5 and SEQ ID NO:28 respectively;
(ix) SEQ ID NO:25 and SEQ ID NO:6 respectively;
(x) SEQ ID NO:25 and SEQ ID NO:27 respectively;
(xi) SEQ ID NO:25 and SEQ ID NO:28 respectively;
(xii) SEQ ID NO:26 and SEQ ID NO:6 respectively;
(xiii) SEQ ID NO:26 and SEQ ID NO:27 respectively;
(xiv) SEQ ID NO:26 and SEQ ID NO:28 respectively;
(xv) SEQ ID NO:37 and SEQ ID NO:38 respectively;
(xvi) SEQ ID NO:7 and SEQ ID NO:8 respectively;
(xvii) SEQ ID NO:7 and SEQ ID NO:32 respectively;
(xviii) SEQ ID NO:7 and SEQ ID NO:33 respectively;
(xix) SEQ ID NO:7 and SEQ ID NO:34 respectively;
(xx) SEQ ID NO:29 and SEQ ID NO:8 respectively;
(xxi) SEQ ID NO:29 and SEQ ID NO:32 respectively;
(xxii) SEQ ID NO:29 and SEQ ID NO:33 respectively;
(xxiii) SEQ ID NO:29 and SEQ ID NO:34 respectively;
(xxiv) SEQ ID NO:30 and SEQ ID NO:8 respectively;
(xxv) SEQ ID NO:30 and SEQ ID NO:32 respectively;
(xxvi) SEQ ID NO:30 and SEQ ID NO:33 respectively;
(xxvii) SEQ ID NO:30 and SEQ ID NO:34 respectively;
(xxviii) SEQ ID NO:31 and SEQ ID NO:8 respectively;
(xxix) SEQ ID NO:31 and SEQ ID NO:32 respectively;
(xxx) SEQ ID NO:31 and SEQ ID NO:33 respectively;
(xxxi) SEQ ID NO:31 and SEQ ID NO:34 respectively;
(xxxii) SEQ ID NO:39 and SEQ ID NO:40 respectively;
(xxxiii) SEQ ID NO:9 and SEQ ID NO:10 respectively;
(xxxiv) SEQ ID NO:11 and SEQ ID NO:12 respectively;
(xxxv) SEQ ID NO:13 and SEQ ID NO:14 respectively;
(xxxvi) SEQ ID NO:15 and SEQ ID NO:16 respectively;
(xxxvii) SEQ ID NO:19 and SEQ ID NO:20 respectively;
(xxxviii) SEQ ID NO:21 and SEQ ID NO:22 respectively;
(xxxix) SEQ ID NO:23 and SEQ ID NO:24 respectively;
(xl) SEQ ID NO:41 and SEQ ID NO:42 respectively;
(xli) SEQ ID NO:43 and SEQ ID NO:44 respectively;
(xlii) SEQ ID NO:45 and SEQ ID NO:46 respectively;
(xliii) SEQ ID NO:47 and SEQ ID NO:48 respectively;
(xliv) SEQ ID NO:49 and SEQ ID NO:50 respectively;
(xlv) SEQ ID NO:51 and SEQ ID NO:44 respectively;
(xlvi) SEQ ID NO:53 and SEQ ID NO:52 respectively;
(xlvii) SEQ ID NO:55 and SEQ ID NO:54 respectively;
(xlviii) SEQ ID NO:57 and SEQ ID NO:56 respectively; and
(xlix) SEQ ID NO:59 and SEQ ID NO:58 respectively.

In some embodiments, the disclosure provides a pharmaceutical composition comprising the any nanostructure described herein, and a pharmaceutically acceptable diluent.

In some embodiments, the disclosure provides a vaccine comprise of any nanostructure described herein.

In some embodiments, the disclosure provides a method of treating or preventing a disease or disorder in a subject in need thereof, comprising administering an effective amount of a nanostructure described herein, a pharmaceutical composition described herein, or a vaccine described herein to the subject.

In some embodiments, the disease or disorder is a viral infection.

In some embodiments, the disclosure provides a method of generating an immune response in a subject in need thereof, comprising administering an effective amount a nanostructure described herein, a pharmaceutical composition described herein, or a vaccine described herein.

In some embodiments, the disclosure provides a kit comprising a nanostructure described herein, a pharmaceutical composition described herein, or a vaccine described herein.

In some embodiments, the disclosure describes use of a nanostructure described herein, a pharmaceutical composition described herein, or a vaccine described herein for the method of any method described herein or as a medicament.

In some embodiments, the disclosure provides a nanostructure described herein, a pharmaceutical composition described herein, or a vaccine described herein for use in a method of described herein or as a medicament.

### BRIEF DESCRIPTION OF THE DRAWINGS

FIG. 1A shows an illustrative embodiment of a protein-based virus-like particle (pbVLP) according to the present disclosure. A nanoparticle pentamer generated in E. coli is combined with a fusion of an antigen and a trimer to assemble into a pbVLP.

FIG. 1B shows further illustrative embodiments of pbVLPs and pbVLP components (with G protein not shown).

FIG. 2 shows SDS-PAGE of soluble and insoluble fractions for E. coli strain screening. Strains ICXBO1-ICBX025, ICXB-027, and ICXB-028 were run on SDS-PAGE. CBM179, CBM163, and CBM181 represent control E. coli strains that have not been transformed.

FIG. 3 shows a chromatogram of a representative DEAE Sepharose purification run of the supernatant collected from inclusion bodies generated in Example 1.

FIG. 4 shows a chromatogram of a representative CHT media purification run.

FIG. 5 shows non-reducing SDS-PAGE of I53-50B extractions with increasing urea concentration. The I53-50B begins to extract at 50 mM urea and ending with 8M urea.

FIG. 6 shows non-reducing SDS PAGE of Inclusion Body wash steps. Test samples were washed with one of the indicated wash buffers, all in a PBS background. The control did not include a wash step but proceeded directly to extraction. IPA=isopropanol

FIG. 7 shows assembly of icosahedral nanostructures (11.4 minutes. retention time) by gel chromatography. Excess compA runs at 17.3 minutes. Non-assembled compB (if present) runs at 22 min

FIG. 8 shows non-reducing SDS PAGE of assembled nanostructures.50A-OG=compA stock (not fused to an antigen); 20181112=compB stock (not fused to an antigen); Test Assembly=SEC purified fraction; 20181030=compB; 1030 Test Assembly=compB; 1008 pool=compB.

FIG. 9 shows a flow diagram of an embodiment of the methods disclosed herein for expressing a protein, harvesting the host cells, lysing the cells, isolating and washing inclusion bodies, and purifying the protein.

FIG. 10 shows a nearest-neighbor joining tree of compA and compB proteins.

FIG. 11 shows a flow diagram of an embodiment for manufacturing compB. MCB=Master Cell Bank (Lot No. 20-0158, Part No. 712801

FIG. 12 shows an SDS-PAGE analysis of I53-dn5A expression in E. coli expression strain IVXB30. Following transformation of E. coli strain CBM179 with pCYT13 containing the I53-dn5A reading frame, production of I53-dn5A was evaluated under control of the phoA promoter. CBM179: Control E. coli strain lacking the I53-dn5A reading frame; IVXB30: E. coli production strain containing the I53-dn5A reading frame; Sol: Soluble fraction generated following harvest of E. coli production cultures; Insol: Insoluble fraction generated following harvest of E. coli production cultures.

### DETAILED DESCRIPTION OF THE INVENTION

The present invention relates to a process for the recombinant production of a component B (compB) protein intended for use in assembling a protein-based Virus-Like Particle (pbVLP).

Unlike known methods for expression and purification of compB proteins, the methods of the present disclose achieve production of compB protein in high yield and purity from the insoluble fraction (inclusion bodies) of a recombinant protein expression system. Previous methods required concentrations of solubilizing agents which resulted in the partial denaturation and refolding of proteins in the inclusion body. As shown herein, compB proteins can be expressed in bacterial cells and purified from inclusion bodies. The methods demonstrate that purification does no require denaturing or refolding steps of the compB protein after solubilization.

### Definitions

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

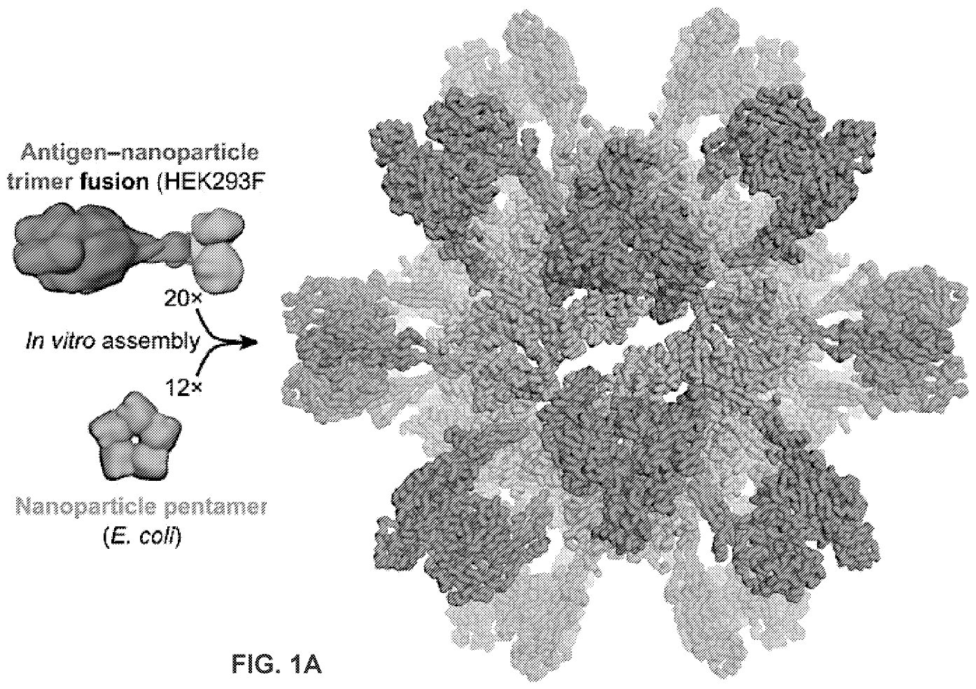

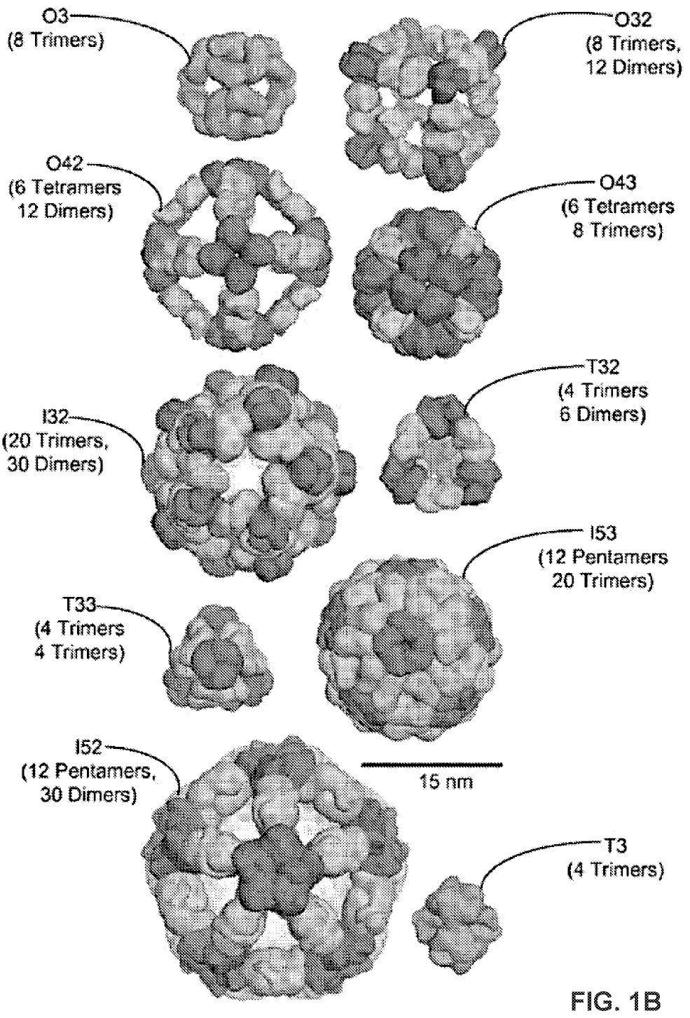

The term “virus-like particle” or “VLP” refers to a molecular assembly that resembles a virus but is non-infectious that displays an antigenic protein, or antigenic fragment thereof, of a viral protein or glycoprotein. A “protein-based VLP” refers to a VLP formed from proteins or glycoproteins and substantially free of other components (e.g., lipids). Protein-based VLPs may include post-translation modification and chemical modification, but are to be distinguished from micellar VLPs and VLPs formed by extraction of viral proteins from live or live inactivated virus preparations. The term “designed VLP” refers to a VLP comprising one or more polypeptides generated by computational protein design. Illustrative designed VLP are VLPs that comprise nanostructures depicted in FIG. 1B. The term “symmetric VLP” refers to a protein-based VLP with a symmetric core, such as shown in FIG. 1B. These include but are not limited to designed VLPs. For example, the protein ferritin has been used to generate a symmetric, protein-based VLP using naturally occurring ferritin sequences. Ferritin-based VLPs are distinguished from designed VLPs in that no protein engineering is necessary to form a symmetric VLP from ferritin, other than fusing the viral protein to the ferritin molecule. Protein design methods can be used to generate similar one- and two-component nanostructures based on template structures (e.g., structures deposited in the Protein Data Bank) or de novo (i.e., by computational design of new proteins having a desired structure but little or no homology to naturally occurring proteins). Such one- and two-component nanostructures can then be used as the core of a designed VLP.

The term “icosahedral particle” refers to a designed pbVLP having a core with icosahedral symmetry (e.g., the particles labeled I53 and I52 in Table 1). I53 refers to an icosahedral particle constructed from pentamers and trimers. I52 refers to an icosahedral particle constructed from pentamers and dimers. T33 refers to a tetrahedral particle constructed from two sets of trimers. T32 refers to a tetrahedral particle constructed from trimers and dimers.

The term “polypeptide” refers to a series of amino acid residues joined by peptide bonds and optionally one or more post-translational modifications (e.g., glycosylation) and/or other modifications (including but not limited to conjugation of the polypeptide moiety used as a marker—such as a fluorescent tag—or an adjuvant).

The term “variant” refers to a polypeptide having one or more insertions, deletions, or amino acid substitutions relative to a reference polypeptide, but retains one or more properties of the reference protein.

The term “functional variant” refers to a variant that exhibits the same or similar functional effect(s) as a reference polypeptide. For example, a functional variant of a multimerization domain is able to promote multimerization to the same extent, or to similar extent, as a reference multimerization domain and/or is able to multimerize with the same cognate multimerization domains as a reference multimerization domain.

The term “fragment” refers to a polypeptide having one or more N-terminal or C-terminal truncations compared to a reference polyp eptide.

The term “functional fragment” refers to a functional variant of a fragment.

The term “amino acid substitution” refers to replacing a single amino acid in a sequence with another amino acid residue. The standard form of abbreviations for amino acid substitution are used. For example, V94R refers to substitution of valine (V) in a reference sequence with arginine (R). The abbreviation Arg94 refers to any sequence in which the 94 th residue, relative to a reference sequence, is arginine (Arg).

The terms “helix” or “helical” refer to an α-helical secondary structure in a polypeptide that is known to occur, or predicted to occur. For example, a sequence may be described as helical when computational modeling suggests the sequence is likely to adopt a helical conformation.

The term “component” refers to a protein, or protein complex, capable of assembly into a virus-like particle under appropriate conditions (e.g., an antigen or polypeptide comprising a multrimerization domain). “Component A” or “compA” and “Component B” or “compB” refer to two proteins capable of assembling to form a pbVLP as described herein. CompA and compB are capable of independently forming dimer, trimer, or pentamer structures as described herein for use in assembly of the pbVLP. In some embodiments, compA is linked to an antigen to form a fusion protein.

The term “pharmaceutically acceptable excipients” means excipients biologically or pharmacologically compatible for in vivo use in animals or humans, and can mean excipients approved by a regulatory agency of the Federal or a state government or listed in the U.S. Pharmacopeia or other generally recognized pharmacopeia for use in animals, and more particularly in humans.

The term “manufacturing” refers to production of a recombinant polypeptide or virus-like particle at any scale, including at least 25-mL, 50-mL, 1-L, 1,000-L, 50,000-L, or greater scale.

The terms “culturing” and “culture medium” refers to standard cell culture and recombinant protein expression techniques.

The term “host cell” refers to any cell capable of use in expression of a recombinant polypeptide.

The term “mixing” refers to placing two solutions into contact to permit the solutions to mix.

The term “purify” refers to separating a molecule from other substances present in a composition. Polypeptides may be purified by affinity (e.g., to an antibody or to a tag, e.g., using a His-tag capture resin), by charge (e.g., ion-exchange chromatography), by size (e.g., preparative ultracentrifugation, size exclusion chromatography), or otherwise.

The terms “polynucleotide” and “nucleic acid,” used interchangeably herein, refer to a polymeric form of nucleotides of more than about 100 nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The terms “identity”, “identical”, and “sequence identity” refer to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

Methods of sequence alignment for comparison and determination of percent sequence identity is well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof

As used herein, the term “denature” refers to a change in the structure of a folded polypeptide molecule that causes the polypeptide to lose all or substantially all tertiary structure, or in the case of a misfolded protein, to convert from an aggregated form into a soluble, unfolded form. The term “denatured” also refers a biologically inactive form of the expressed protein, as obtained as a product of the recombinant production process, after solubilizing inclusion bodies under conditions under which the native three-dimensional structure of the protein is disrupted.

The term “refolding” (or “renaturing”) refers to a process that causes a denatured protein to regain its native conformation and biological activity.

As used herein, the term “recovering” refers to obtaining a substance (e.g. inclusion bodies and/or a protein of interest) by separating the substance from other substances in a preparation, e.g., by centrifugation and/or one or more wash steps.

As used herein, the term “inclusion body” refers to insoluble aggregates containing recombinant protein present in the cytoplasm of transformed host cells. These appear as bright spots under the microscope and can be recovered by separation of inclusion bodies from the cytoplasm of the cell. In the prior art, inclusion bodies are typically solubilized using high concentrations of a chaotropic agent (e.g. >8 M urea and/or >3 M guanidinium); a strong ionic detergent (e.g., N-lauroylsarcosine); and/or alkaline pH. According to the present disclosure, lower concentrations of these agents may be used to gently solubilize.

As used herein, the term “nanostructure” include symmetrically repeated, non-natural, non-covalent protein-protein interfaces that orient a first component molecule (e.g. compA) and a second component molecule (e.g. compB) into an assembled structure. Nanostructures include but are not limited to delivery vehicles, as the nanostructures can encapsulate molecules of interest and/or the first and/or second proteins can be modified to bind to molecules of interest (diagnostics, therapeutics, detectable molecules for imaging and other applications, etc.). The nanostructures of the disclosure are well suited for several applications, including vaccine design, targeted delivery of therapeutics, and bioenergy.

As used herein, the term “solubilization” refers to a transfer of proteins comprised within a biological sample to a solvent such as an aqueous solvent by disrupting the cells of the biological sample. As used herein, “solubilization” or “solubilize” may be used interchangeably with “to dissolve” or “to extract”. The term “solubilization” also refers to the release of a protein from inclusion bodies, e.g., by dissolving the inclusion bodies.

The following description includes information that may be useful in understanding the present invention. It is not an admission that any of the information provided herein is prior art or relevant to the presently claimed inventions, or that any publication specifically or implicitly referenced is prior art.

### Embodiments

The present disclosure demonstrates expression of component B (compB) protein for a protein-based VLP from inclusion bodies. Surprisingly, the compB protein produced according to the methods of the disclosure is at least as soluble and assembly-competent, or more so, than compB protein expressed into and purified from the soluble fraction of the host cell.

Vaccination is a treatment modality used to prevent or decrease the severity of infection with various infectious agents, including bacteria, viruses, and parasites. Development of new vaccines has important commercial and public health implications. In particular, lyme disease, pertussis, herpes virus, orthomyxovirus, paramyxovirus, pneumovirus, Filovirus, flavivirus, reovirus, retrovirus, coronavirus, and malaria are infectious agents for which vaccines already exist, are being developed, or would be desirable.

Subunit vaccines are vaccines made from isolated antigens, usually proteins expressed recombinantly in bacterial, insect, or mammalian cell hosts. Typically, the antigenic component of a subunit vaccine is selected from among the proteins of an infectious agent observed to elicit a natural immune response upon infection, although in some cases other components of the infectious agent can be used. Typical antigens for use in subunit vaccines include protein expressed on the surface of the target infectious agent, as such surface-expressed envelope glycoproteins of viruses. Preferably, the antigen is a target for neutralizing antibodies. More preferably, the antigen is a target for broadly neutralizing antibodies, such that the immune response to the antigen covers immunity against multiple strains of the infectious agent. In some cases, glycans that are N-linked or O-linked to the subunit vaccine may also be important in vaccination, either by contributing to the epitope of the antigen or by guiding the immune response to particular epitopes on the antigen by steric hindrance. The immune response that occurs in response to vaccination may be direct to the protein itself, to the glycan, or to both the protein and linked glycans. Subunit vaccines have various advantages including that they contain no live pathogen, which eliminates concerns about infection of the patient by the vaccine; they may be designed using standard genetic engineering techniques; they are more homogenous than other forms of vaccine; and they can be manufactured in standardized recombinant protein expression production systems using well-characterized expression systems. In some cases, the antigen may be genetically engineered to favor generation of desirable antibodies, such as neutralizing or broadly neutralizing antibodies. In particular, structural information about an antigen of interest, obtained by X-ray crystallography, electron microscopy, or nuclear magnetic resonance experiments, can be used to guide rational design of subunit vaccines.

A known limitation of subunit vaccines is that the immune response elicited may sometimes be weaker than the immune response to other types of vaccines, such as whole virus, live, or live-attenuated vaccines. Designed and/or protein-based VLP vaccines have the potential to harness the advantages of subunit vaccines while increasing the potency and breadth of the vaccine-induced immune response through multivalent display of the antigen in symmetrically ordered arrays. In the present disclosure, protein-based VLPs are distinguished from nanoparticle vaccines, because the term nanoparticle vaccine has been used in the art to refer to protein-based or glycoprotein-based vaccines (see, e.g. U.S. Pat. No. 9,441,019), polymerized liposomes (see, e.g., U.S. Pat. No. 7,285,289), surfactant micelles (see, e.g., US Patent Pub. No. US 2004/0038406 A1), and synthetic biodegradable particles (see, e.g., U.S. Pat. No. 8,323,696).

A non-limiting example of an embodiment is shown in FIG. 1A, which depicts a protein antigen genetically fused to a component A (compA) protein of the pbVLP, which optionally is expressed recombinantly in a host cell (e.g., 293F cells); along with a component B (compB) protein assembly, which is expressed recombinantly in a host cell (e.g., E. coli cells), these two components self-assembling into a pbVLP displaying 20 copies of the protein antigen around an icosahedral core.

In some embodiments, compA is a dimer. In some embodiments, compA is a trimer. In some embodiments, compA is a pentamer.

In some embodiments, compB is a dimer. In some embodiments, compB is a trimer. In some embodiments, compA is a pentamer.

In some embodiments, compA is a dimer selected from SEQ ID Nos: 13,17, or 41. In some embodiments, compA is a trimer selected from SEQ ID Nos: 5, 7, 9, 19, 21, 25, 26, 29, 30, 31, 37, 39, 43, 45, 47, 49, or 51. In some embodiments, compA is a pentamer selected from SEQ ID Nos: 3, 11, 15, 23, or 35.

In some embodiments, compB is a dimer selected from SEQ ID Nos: 12, 16, 20, or 22. In some embodiments, compB is a trimer selected from SEQ ID Nos: 4, 18, 24, 34, 36, 42, 44, 46, 48, or 50. In some embodiments, compB is a pentamer selected from SEQ ID Nos: 2, 6, 8, 10, 14, 27, 28, 32, 33, 38, or 40.

In some embodiments, compA comprises a polypeptide sequence that has at least 90%, at least 95%,at least at least 99%, or 100% identity to any one of SEQ IN NOs: 1, 3, 5, 7, 9, 11, 13, 15, 17, 19, 21, 23, 25, 26, 29-31, 35, 37, 39, 41, 43, 45, 47, 49, 51, 53, 55, 57, and 59.

In some embodiments, compB comprises a polypeptide sequence that has at least 90%, at least 95%, at least 99%, or 100% identity to any one of SEQ IN NOs: 2, 4, 6, 8, 10, 12, 14, 16, 18, 20, 22, 24, 27, 28, 32-34, 36, 38, 40, 42, 44, 46, 48, 50, 52, 54, 56, and 58.

In some embodiments, compA and Comp B form an “I53” architecture. An I53 architecture is a combination of 12 pentameric building blocks and twenty trimeric building blocks aligned along the five-fold and three-fold icosahedral symmetry axes as described in Bale et al., Science 353:389-394 (2016). In some embodiments, compA is the I53 pentamer. In some embodiments, compA is the I53 trimer. In some embodiments, compB is the I53 pentamer. In some embodiments, compB is the I53 trimer.

In some embodiments, compA and compB form an “I52” architecture. An I52 architecture is formed from twelve pentamers and thirty dimers along their corresponding icosahedral symmetry axes. In some embodiments, compA is the I52 pentamer. In some embodiments, compA is the I52 dimer. In some embodiments, compB is the I52 pentamer. In some embodiments, compB is the I52 dimer.

In some embodiments, compA and compB form an “I32” architecture. An I32 architecture is a combination of twenty trimers and thirty dimers, each aligned along their corresponding icosahedral symmetry axes. In some embodiments, compA is the I32 trimer. In some embodiments, compA is the I32 dimer. In some embodiments, compB is the I32 trimer. In some embodiments, compB is the I32 dimer.

In some embodiments, a mixture of compA and compB forms an icosahedral nanostructure. In some embodiments, a mixture of compA and compB forms a tetrahedral nanostructure. In some embodiments, a mixture of compA and compB forms an octahedral nanostructure.

In some embodiments, a small-molecule drug (i.e., with MW of less than 700), biological drug (i.e., drugs isolated from a bacterium, yeast, cell, or organ, especially including recombinant polypeptides), or biosynthetic drugs (e.g., aptamers, antisense nucleic acid, siRNA, recombinant nucleic acid, nucleoside analogs, recombinant polypeptides, polypeptide drugs, antigens, etc) is fused to compA.

In some embodiments, an antigen is fused to compA

In some embodiments, compA and compB form a nanostructure.

In some embodiments, the nanostructure is formed by combining a compA selected from one of SEQ ID NOs: 1, 3, 5, 7, 9, 11, 13, 15, 17, 19, 21, 23, 25, 26, 29-31, 35, 37, 39, 41, 43, 45, 47, 49, 51, 53, 55, 57, 59, and a compB selected from one of SEQ ID NOs: 2, 4, 6, 8, 10, 12, 14, 16, 18, 20, 22, 24, 27, 28, 32-34, 36, 38, 40, 42, 44, 46, 48, 50, 52, 54, 56, 58.

In some embodiments, the nanostructure is formed from a compA and compB polypeptide sequence that has at least 90%, at least 95%, at least 99%, or 100% identity to any one of;
(i) SEQ ID NO:1 and SEQ ID NO:2 respectively;
(ii) SEQ ID NO:3 and SEQ ID NO:4 respectively;
(iii) SEQ ID NO:3 and SEQ ID NO:24 respectively;
(iv) SEQ ID NO:23 and SEQ ID NO:4 respectively;
(v) SEQ ID NO:35 and SEQ ID NO:36 respectively;
(vi) SEQ ID NO:5 and SEQ ID NO:6 respectively;
(vii) SEQ ID NO:5 and SEQ ID NO:27 respectively;
(viii) SEQ ID NO:5 and SEQ ID NO:28 respectively;
(ix) SEQ ID NO:25 and SEQ ID NO:6 respectively;
(x) SEQ ID NO:25 and SEQ ID NO:27 respectively;
(xi) SEQ ID NO:25 and SEQ ID NO:28 respectively;
(xii) SEQ ID NO:26 and SEQ ID NO:6 respectively;
(xiii) SEQ ID NO:26 and SEQ ID NO:27 respectively;
(xiv) SEQ ID NO:26 and SEQ ID NO:28 respectively;
(xv) SEQ ID NO:37 and SEQ ID NO:38 respectively;
(xvi) SEQ ID NO:7 and SEQ ID NO:8 respectively;
(xvii) SEQ ID NO:7 and SEQ ID NO:32 respectively;
(xviii) SEQ ID NO:7 and SEQ ID NO:33 respectively;
(xix) SEQ ID NO:7 and SEQ ID NO:34 respectively;
(xx) SEQ ID NO:29 and SEQ ID NO:8 respectively;
(xxi) SEQ ID NO:29 and SEQ ID NO:32 respectively;
(xxii) SEQ ID NO:29 and SEQ ID NO:33 respectively;
(xxiii) SEQ ID NO:29 and SEQ ID NO:34 respectively;
(xxiv) SEQ ID NO:30 and SEQ ID NO:8 respectively;
(xxv) SEQ ID NO:30 and SEQ ID NO:32 respectively;
(xxvi) SEQ ID NO:30 and SEQ ID NO:33 respectively;
(xxvii) SEQ ID NO:30 and SEQ ID NO:34 respectively;
(xxviii) SEQ ID NO:31 and SEQ ID NO:8 respectively;
(xxix) SEQ ID NO:31 and SEQ ID NO:32 respectively;
(xxx) SEQ ID NO:31 and SEQ ID NO:33 respectively;
(xxxi) SEQ ID NO:31 and SEQ ID NO:34 respectively;
(xxxii) SEQ ID NO:39 and SEQ ID NO:40 respectively;
(xxxiii) SEQ ID NO:9 and SEQ ID NO:10 respectively;
(xxxiv) SEQ ID NO:11 and SEQ ID NO:12 respectively;
(xxxv) SEQ ID NO:13 and SEQ ID NO:14 respectively;
(xxxvi) SEQ ID NO:15 and SEQ ID NO:16 respectively;
(xxxvii) SEQ ID NO:19 and SEQ ID NO:20 respectively;
(xxxviii) SEQ ID NO:21 and SEQ ID NO:22 respectively;
(xxxix) SEQ ID NO:23 and SEQ ID NO:24 respectively;
(xl) SEQ ID NO:41 and SEQ ID NO:42 respectively;
(xli) SEQ ID NO:43 and SEQ ID NO:44 respectively;
(xlii) SEQ ID NO:45 and SEQ ID NO:46 respectively;
(xliii) SEQ ID NO:47 and SEQ ID NO:48 respectively;
(xliv) SEQ ID NO:49 and SEQ ID NO:50 respectively;
(xlv) SEQ ID NO:51 and SEQ ID NO:44 respectively;
(xlvi) SEQ ID NO:53 and SEQ ID NO:52 respectively;
(xlvii) SEQ ID NO:55 and SEQ ID NO:54 respectively;
(xlviii) SEQ ID NO:57 and SEQ ID NO:56 respectively; and
(xlix) SEQ ID NO:59 and SEQ ID NO:58 respectively.

In some embodiments, compA comprises a helical extension. In some embodiments, the helical extension is located at the N-terminus of compA. In some embodiments, the helical extension is EKAAKAEEAARK (SEQ ID NO: 62).

In some embodiments, compB comprises a helical extension. In some embodiments, the helical extension is located at the N-terminus of compB. In some embodiments, the helical extension is EKAAKAEEAARK (SEQ ID NO: 62).

In some embodiments, the nanostructure is a pbVLP. In some embodiments, the pbVLP is a vaccine.

Other compA/compB assemblies of the present disclosure are shown in FIG. 1B. In some embodiments, the pbVLP is adapted for display of up to 8 trimers; 8 trimers and 12 dimers; 6 tetramers and 12 dimers; 6 tetramers and 8 trimers; 20 trimers and 30 dimers; 4 trimers and 6 dimers; 4 first trimers and 4 second trimers, or 8 trimers; 12 pentamers and 20 trimers; or 12 pentamers and 30 dimers; or 4 trimers. In some cases, one of the symmetric axes is not used for antigen display, thus, in some embodiments the pbVLP is adapted for display of up to 8 trimers; 12 dimers; 6 tetramers; 20 trimers; 30 dimers; 4 trimers; 6 dimers; 8 trimers; or 12 pentamers. In some cases, monomeric antigens are displayed and thus, the pbVLP is adapted for display of up to 12, 24, 60, or 70 monomeric antigens. In some cases, the pbVLP comprises mixed pluralities of polypeptides such that otherwise identical polypeptides of the core of the pbVLP display different antigens or no antigen. Thus, depending on the ratio of polypeptides, the pbVLP is in some cases adapted for display of between 1 and 130 antigens (e.g., on the I52 particle) where each of the antigens displayed may be the same or may be different members of mixed population in proportion to any ratio chosen. The antigens may be co-expressed in a recombinant expression system and self-assembled before purification. Non-limiting exemplary pbVLPs are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

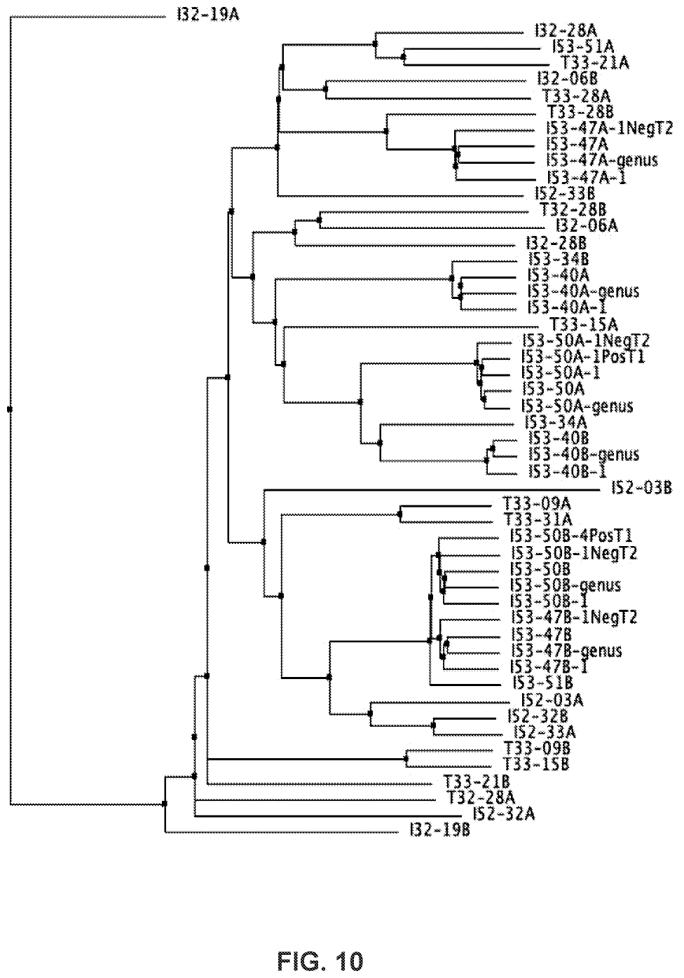

The compA and compB proteins of the present disclosure may have any of various amino acids sequences. U.S. Patent Pub No. US 2015/0356240 A1 describes various methods for designing protein assemblies. As described in US Patent Pub No. US 2016/0122392 A1 and in International Patent Pub. No. WO 2014/124301 A1, the isolated polypeptides of SEQ ID NOS:1-51 were designed for their ability to self-assemble in pairs to form pbVLPs, such as icosahedral particles. The design involved design of suitable interface residues for each member of the polypeptide pair that can be assembled to form the pbVLP. The pbVLPs so formed include symmetrically repeated, non-natural, non-covalent polypeptide-polypeptide interfaces that orient a first assembly and a second assembly into a pbVLP, such as one with an icosahedral symmetry. Thus, in some embodiments the compA and compBs (that is, the two polypeptides of the core of the pbVLP) are selected from the group consisting of SEQ ID NOS:1-51. In each case, an N-terminal methionine residue present in the full-length protein but typically removed to make a fusion is not included in the sequence. In various embodiments, one or more additional residues are deleted from the N-terminus and/or additional residues are added to the N-terminus (e.g. to form a helical extension). As shown in FIG. 10, the sequences disclosed below group into several families of related protein sequences.

TABLE 1

| | | | Identified |
|---|---|---|---|
| | Component | | interface |
| Name | Multrimer | Amino Acid Sequence | residues |
| I53-34A | trimer | EGMDPLAVLAESRLLPLLTVRGGEDLAGLA | I53-34A: |
| SEQ ID NO: 1 | | TVLELMGVGALEITLRTEKGLEALKALRKS | 28, |
| | | GLLLGAGTVRSPKEAEAALEAGAAFLVSPG | 32, 36, 37, |
| | | LLEEVAALAQARGVPYLPGVLTPTEVERAL | 186, 188, |
| | | ALGLSALKFFPAEPFQGVRVLRAYAEVFPE | 191, 192, 195 |
| | | VRFLPTGGIKEEHLPHYAALPNLLAVGGSW | |
| | | LLQGDLAAVMKKVKAAKALLSPQAPG | |
| I53-34B | pentamer | TKKVGIVDTTFARVDMAEAAIRTLKALSPN | I53-34B: |
| SEQ ID NO: 2 | | IKIIRKTVPGIKDLPVACKKLLEEEGCDIV | 19, |
| | | MALGMPGKAEKDKVCAHEASLGLMLAQLMT | 20, 23, 24, |
| | | NKHIIEVFVHEDEAKDDDELDILALVRAIE | 27, 109, |
| | | HAANVYYLLFKPEYLTRMAGKGLRQGREDA | 113, 116, 117, |
| | | GPARE | 120, 124, |
| | | | 148 |
| I53-40A | pentamer | TKKVGIVDTTFARVDMASAAILTLKMESPN | I53-40A: |
| SEQ ID NO: 3 | | IKIIRKTVPGIKDLPVACKKLLEEEGCDIV | 20, |
| | | MALGMPGKAEKDKVCAHEASLGLMLAQLMT | 23, 24, 27, |
| | | NKHIIEVFVHEDEAKDDAELKILAARRAIE | 28, 109, 112, |
| | | HALNVYYLLFKPEYLTRMAGKGLRQGFEDA | 113, 116, |
| | | GPARE | 120, 124 |
| I53-40B | trimer | STINNQLKALKVIPVIAIDNAEDIIPLGKV | I53-40B: |
| SEQ ID NO: 4 | | LAENGLPAAEITFRSSAAVKAIMLLRSAQP | 47, |
| | | EMLIGAGTILNGVQALAAKEAGATFVVSPG | 51, 54, 58, |
| | | FNPNTVRACQIIGIDIVPGVNNPSTVEAAL | 74, 102 |
| | | EMGLTTLKFFPAEASGGISMVKSLVGPYGD | |
| | | IRLMPTGGITPSNIDNYLAIPQVLACGGTW | |
| | | MVDKKLVTNGEWDEIARLTREIVEQVNP | |
| I53-47A | trimer | PIFTLNTNIKATDVPSDFLSLTSRLVGLIL | I53-47A: |
| SEQ ID NO: 5 | | SKPGSYVAVHINTDQQLSFGGSTNPAAFGT | 22, |
| | | LMSIGGIEPSKNRDHSAVLFDHLNAMLGIP | 25, 29, 72, |
| | | KNRMYIHFVNLNGDDVGWNGTTF | 79, 86, 87 |
| I53-47B | pentamer | NQHSHKDYETVRIAVVRARWHADIVDACVE | I53-47B: |
| SEQ ID NO: 6 | | AFEIAMAAIGGDRFAVDVEDVPGAYEIPLH | 28, |
| | | ARTLAETGRYGAVLGTAFVVNGGIYRHEFV | 31, 35, 36, |
| | | ASAVIDGMMNVQLSTGVPVLSAVLTPHRYR | 39, 131, 132, |
| | | DSAEHHRFFAAHFAVKGVEAARACIEILAA | 135, 139, |
| | | REKIAA | 146 |
| I53-50A | trimer | MEELFKKHKIVAVLRANSVEEAIEKAVAVF | I53-50A: |
| SEQ ID NO: 7 | | AGGVHLIEITFTVPDADTVIKALSVLKEKG | 25, |
| | | AIIGAGTVTSVEQCRKAVESGAEFIVSPHL | 29, 33, 54, |
| | | DEEISQFCKEKGVFYMPGVMTPTELVKAMK | 57 |
| | | LGHTILKLFPGEVVGPQFVKAMKGPFPNVK | |
| | | FVPTGGVNLDNVCEWFKAGVLAVGVGSALV | |
| | | KGTPDEVREKAKAFVEKIRGCTE | |
| I53-50B | pentamer | NQHSHKDYETVRIAVVRARWHAEIVDACVS | I53-50B: |
| SEQ ID NO: 8 | | AFEAAMADIGGDRFAVDVEDVPGAYEIPLH | 24, |
| | | ARTLAETGRYGAVLGTAFVVNGGIYRHEFV | 28, 36, 124, |
| | | ASAVIDGMMNVQLSTGVPVLSAVLTPHRYR | 125, 127, |
| | | DSDAHTLLFLALFAVKGMEAARACVEILAA | 128, 129, |
| | | REKIAA | 131, 132, 133, |
| | | | 135, 139 |
| I53-51A | trimer | FTKSGDDGNTNVINKRVGKDSPLVNFLGDL | I53-51A: |
| SEQ ID NO: 9 | | DELNSFIGFAISKIPWEDMKKDLERVQVEL | 80, |
| | | FEIGEDLSTQSSKKKIDESYVLWLLAATAI | 83, 86, 87, |
| | | YRIESGPVKLFVIPGGSEEASVLHVTRSVA | 88, 90, 91, |
| | | RRVERNAVKYTKELPEINRMIIVYLNRLSS | 94, 166, 172, |
| | | LLFAMALVANKRRNQSEKIYEIGKSW | 176 |
| I53-51B | pentamer | NQHSHKDYETVRIAVVRARWHADIVDQCVR | I53-51B: |
| SEQ ID | | AFEEAMADAGGDRFAVDVFDVPGAYEIPLH | 31, |
| NO: 10 | | ARTLAETGRYGAVLGTAFVVNGGIYRHEFV | 35, 36, 40, |
| | | ASAVIDGMMNVQLSTGVPVLSAVLTPHRYR | 122, 124, 1 |
| | | SSREHHEFFREHFMVKGVEAAAACITILAA | 28, 131, 135, |
| | | REKIAA | 139, 143, |
| | | | 146, 147 |
| I52-03A | pentamer | GHTKGPTPQQHDGSALRIGIVHARWNKTII | I52-03A: |
| SEQ ID | | MPLLIGTIAKLLECGVKASNIVVQSVPGSW | 28, |
| NO: 11 | | ELPIAVQRLYSASQLQTPSSGPSLSAGDLL | 32, 36, 39, |
| | | GSSTTDLTALPTTTASSTGPFDALIAIGVL | 44, 49 |
| | | IKGETMHFEYIADSVSHGLMRVQLDTGVPV | |
| | | IFGVLTVLTDDQAKARAGVIEGSHNHGEDW | |
| | | GLAAVEMGVRRRDWAAGKTE | |
| I52-03B | dimer | YEVDHADVYDLFYLGRGKDYAAEASDIADL | I52-03B: |
| SEQ ID | | VRSRTPEASSLLDVACGTGTHLEHFTKEFG | 94, |
| NO: 12 | | DTAGLELSEDMLTHARKRLPDATLHQGDMR | 115, 116, |
| | | DFQLGRKFSAVVSMFSSVGYLKTVAELGAA | 206, 213 |
| | | VASFAEHLEPGGVVVVEPWWFPETFADGWV | |
| | | SADVVRRDGRTVARVSHSVREGNATRMEVH | |
| | | FTVADPGKGVRHFSDVHLITLFHQREYEAA | |
| | | FMAAGLRVEYLEGGPSGRGLFVGVPA | |
| I52-32A | dimer | GMKEKFVLIITHGDFGKGLLSGAEVIIGKQ | I52-32A: |
| SEQ ID | | ENVHTVGLNLGDNIEKVAKEVMRIIIAKLA | 47, |
| NO: 13 | | EDKEIIIVVDLFGGSPFNIALEMMKTFDVK | 49, 53, 54, |
| | | VITGINMPMLVELLTSINVYDTTELLENIS | 57, 58, 61, |
| | | KIGKDGIKVIEKSSLKM | 83, 87, 88 |
| I52-32B | pentamer | KYDGSKLRIGILHARWNLEIIAALVAGAIK | I52-32B: |
| SEQ ID | | RLQEFGVKAENIIIETVPGSFELPYGSKLF | 19, |
| NO: 14 | | VEKQKRLGKPLDAIIPIGVLIKGSTMHFEY | 20, 23, 30, |
| | | ICDSTTHQLMKLNFELGIPVIFGVLTCLTD | 40 |
| | | EQAEARAGLIEGKMHNHGEDWGAAAVEMAT | |
| | | KEN | |
| I52-33A | pentamer | AVKGLGEVDQKYDGSKLRIGILHARWNRKI | I52-33A: |
| SEQ ID | | ILALVAGAVLRLLEFGVKAENIIIETVPGS | 33, |
| NO: 15 | | FELPYGSKLFVEKQKRLGKPLDAIIPIGVL | 41, 44, 50 |
| | | IKGSTMHFEYICDSTTHQLMKLNFELGIPV | |
| | | IFGVLTCLTDEQAEARAGLIEGKMHNHGED | |
| | | WGAAAVEMATKFN | |
| I52-33B | dimer | GANWYLDNESSRLSFTSTKNADIAEVHRFL | I52-33B: |
| SEQ ID | | VLHGKVDPKGLAEVEVETESISTGIPLRDM | 61, |
| NO: 16 | | LLRVLVFQVSKFPVAQINAQLDMRPINNLA | 63, 66, 67, |
| | | PGAQLELRLPLTVSLRGKSHSYNAELLATR | 72, 147, |
| | | LDERRFQVVTLEPLVIHAQDFDMVRAFNAL | 148, 154, 155 |
| | | RLVAGLSAVSLSVPVGAVLIFTAR | |
| I32-06A | dimer | TDYIRDGSAIKALSFAIILAEADLRHIPQD | I32-06A: |
| SEQ ID | | LQRLAVRVIHACGMVDVANDLAFSEGAGKA | 9, |
| NO: 17 | | GRNALLAGAPILCDARMVAEGITRSRLPAD | 12, 13, 14, |
| | | NRVIYTLSDPSVPELAKKIGNTRSAAALDL | 20, 30, 33, |
| | | WLPHIEGSIVAIGNAPTALFRLFELLDAGA | 34 |
| | | PKPALIIGMPVGFVGAAESKDELAANSRGV | |
| | | PYVIVRGRRGGSAMTAAAVNALASERE | |
| I32-06B | trimer | ITVFGLKSKLAPRREKLAEVIYSSLHLGLD | I32-06B: |
| SEQ ID | | IPKGKHAIRFLCLEKEDFYYPFDRSDDYTV | 24, |
| NO: 18 | | IEINLMAGRSEETKMLLIFLLFIALERKLG | 71, 73, 76, |
| | | IRAHDVEITIKEQPAHCWGFRGRTGDSARD | 77, 80, 81, |
| | | LDYDIYV | 84, 85, 88, |
| | | | 114, 118 |
| I32-19A | trimer | GSDLQKLQRFSTCDISDGLLNVYNIPTGGY | I32-19A: |
| SEQ ID | | FPNLTAISPPQNSSIVGTAYTVLFAPIDDP | 208, |
| NO: 19 | | RPAVNYIDSVPPNSILVLALEPHLQSQFHP | 213, 218, |
| | | FIKITQAMYGGLMSTRAQYLKSNGTVVFGR | 222, 225, |
| | | IRDVDEHRTLNHPVFAYGVGSCAPKAVVKA | 226, 229, 233 |
| | | VGTNVQLKILTSDGVTQTICPGDYIAGDNN | |
| | | GIVRIPVQETDISKLVTYIEKSIEVDRLVS | |
| | | EAIKNGLPAKAAQTARRMVLKDYI | |
| I32-19B | dimer | SGMRVYLGADHAGYELKQAIIAFLKMTGHE | I32-19B: |
| SEQ ID | | PIDCGALRYDADDDYPAFCIAAATRTVADP | 20, |
| NO: 20 | | GSLGIVLGGSGNGEQIAANKVPGARCALAW | 23, 24, 27, |
| | | SVQTAALAREHNNAQLIGIGGRMHTLEEAL | |
| | | RIVKAFVTTPWSKAQRHQRRIDILAEYERT | |
| | | HEAPPVPGAPA | 117, 118, 12 |
| | | | 2, 125 |
| I32-28A | trimer | GDDARIAAIGDVDELNSQIGVLLAEPLPDD | I32-28A: |
| SEQ ID | | VRAALSAIQHDLEDLGGELCIPGHAAITED | 60, |
| NO: 21 | | HLLRLALWLVHYNGQLPPLEEFILPGGARG | 61, 64, 67, |
| | | AALAHVCRTVCRRAERSIKALGASEPLNIA | 68, 71, 110, |
| | | PAAYVNLLSDLLFVLARVLNRAAGGADVLW | 120, 123, |
| | | DRTRAH | 124, 128 |
| I32-28B | dimer | ILSAEQSFTLRHPHGQAAALAFVREPAAAL | I32-28B: |
| SEQ ID | | AGVQRLRGLDSDGEQVWGELLVRVPLLGEV | 35, 36, 54, |
| NO: 22 | | DLPFRSEIVRTPQGAELRPLTLTGERAWVA | 122, |
| | | VSGQATAAEGGEMAFAFQFQAHLATPEAEG | 129, 137, |
| | | EGGAAFEVMVQAAAGVTLLLVAMALPQGLA | 140, 141, |
| | | AGLPPA | 144, 148 |
| I53-40A.1 | pentamer | TKKVGIVDTTFARVDMASAAILTLKMESPN | I53-40A: |
| SEQ ID | | IKIIRKTVPGIKDLPVACKKLLEEEGCDIV | 20, |
| NO: 23 | | MALGMPGKKEKDKVCAHEASLGLMLAQLMT | 23, 24, 27, |
| | | NKHIIEVFVHEDEAKDDAELKILAARRAIE | 28, 109, 112, |
| | | HALNVYYLLFKPEYLTRMAGKGLRQGFEDA | 113, 116, |
| | | GPARE | 120, 124 |
| I53-40B.1 | trimer | DDINNQLKRLKVIPVIAIDNAEDIIPLGKV | I53-40B: |
| SEQ ID | | LAENGLPAAEITFRSSAAVKAIMLLRSAQP | 47, |
| NO: 24 | | EMLIGAGTILNGVQALAAKEAGADFVVSPG | 51, 54, 58, |
| | | FNPNTVRACQIIGIDIVPGVNNPSTVEQAL | 74, 102 |
| | | EMGLTTLKFFPAEASGGISMVKSLVGPYGD | |
| | | IRLMPTGGITPDNIDNYLAIPQVLACGGTW | |
| | | MVDKKLVRNGEWDEIARLTREIVEQVNP | |
| I53-47A.1 | trimer | PIFTLNTNIKADDVPSDFLSLTSRLVGLIL | I53-47A: |
| SEQ ID | | SKPGSYVAVHINTDQQLSFGGSTNPAAFGT | 22, |
| NO: 25 | | LMSIGGIEPDKNRDHSAVLFDHLNAMLGIP | 25, 29, 72, |
| | | KNRMYIHFVNLNGDDVGWNGTTF | 79, 86, 87 |
| I53- | trimer | PIFTLNTNIKADDVPSDFLSLTSRLVGLIL | I53-47A: |
| 47A.1NegT2 | | SEPGSYVAVHINTDQQLSFGGSTNPAAFGT | 22, |
| SEQ ID | | LMSIGGIEPDKNEDHSAVLFDHLNAMLGIP | 25, 29, 72, |
| NO: 26 | | KNRMYIHFVDLDGDDVGWNGTTF | 79, 86, 87 |
| I53-47B.1 | pentamer | NQHSHKDHETVRIAVVRARWHADIVDACVE | I53-47B: |
| SEQ ID | | AFEIAMAAIGGDRFAVDVEDVPGAYEIPLH | 28, |
| NO: 27 | | ARTLAETGRYGAVLGTAFVVNGGIYRHEFV | 31, 35, 36, |
| | | ASAVIDGMMNVQLDTGVPVLSAVLTPHRYR | 39, 131, 132, |
| | | DSDEHHRFFAAHFAVKGVEAARACIEILNA | 135, 139, |
| | | REKIAA | 146 |
| I53- | pentamer | NQHSHKDHETVRIAVVRARWHADIVDACVE | I53-47B: |
| 47B.1NegT2 | | AFEIAMAAIGGDRFAVDVFDVPGAYEIPLH | 28, |
| SEQ ID | | ARTLAETGRYGAVLGTAFVVDGGIYDHEFV | 31, 35, 36, |
| NO: 28 | | ASAVIDGMMNVQLDTGVPVLSAVLTPHEYE | 39, 131, 132, |
| | | DSDEDHEFFAAHFAVKGVEAARACIEILNA | 135, 139, |
| | | REKIAA | 146 |
| I53-50A.1 | trimer | EELFKKHKIVAVLRANSVEEAIEKAVAVFA | I53-50A: |
| SEQ ID | | GGVHLIEITFTVPDADTVIKALSVLKEKGA | 25, |
| NO: 29 | | IIGAGTVTSVEQCRKAVESGAEFIVSPHLD | 29, 33, 54, |
| | | EEISQFCKEKGVFYMPGVMTPTELVKAMKL | 57 |
| | | GHDILKLFPGEVVGPQFVKAMKGPFPNVKF | |
| | | VPTGGVNLDNVCEWFKAGVLAVGVGDALVK | |
| | | GDPDEVREKAKKFVEKIRGCTE | |
| I53- | trimer | EELFKKHKIVAVLRANSVEEAIEKAVAVFA | I53-50A: |
| 50A.1NegT2 | | GGVHLIEITFTVPDADTVIKALSVLKEKGA | 25, |
| SEQ ID | | IIGAGTVTSVEQCRKAVESGAEFIVSPHLD | 29, 33, 54, |
| NO: 30 | | EEISQFCKEKGVFYMPGVMTPTELVKAMKL | 57 |
| | | GHDILKLFPGEVVGPEFVEAMKGPFPNVKF | |
| | | VPTGGVDLDDVCEWFDAGVLAVGVGDALVE | |
| | | GDPDEVREDAKEFVEEIRGCTE | |
| I53- | trimer | EELFKKHKIVAVLRANSVEEAIEKAVAVFA | I53-50A: |
| 50A.1PosT1 | | GGVHLIEITFTVPDADTVIKALSVLKEKGA | 25, |
| SEQ ID | | IIGAGTVTSVEQCRKAVESGAEFIVSPHLD | 29, 33, 54, |
| NO: 31 | | EEISQFCKEKGVFYMPGVMTPTELVKAMKL | 57 |
| | | GHDILKLFPGEVVGPQFVKAMKGPFPNVKF | |
| | | VPTGGVNLDNVCKWFKAGVLAVGVGKALVK | |
| | | GKPDEVREKAKKFVKKIRGCTE | |
| I53-50B.1 | pentamer | NQHSHKDHETVRIAVVRARWHAEIVDACVS | I53-50B: |
| SEQ ID | | AFEAAMRDIGGDRFAVDVEDVPGAYEIPLH | 24, |
| NO: 32 | | ARTLAETGRYGAVLGTAFVVNGGIYRHEFV | 28, 36, 124, |
| | | ASAVIDGMMNVQLDTGVPVLSAVLTPHRYR | 125, 127, |
| | | DSDAHTLLFLALFAVKGMEAARACVEILAA | 128, 129, 131, |
| | | REKIAA | 132, 133, |
| | | | 135, 139 |
| I53- | pentamer | NQHSHKDHETVRIAVVRARWHAEIVDACVS | I53-50B: |
| 50B.1NegT2 | | AFEAAMRDIGGDRFAVDVEDVPGAYEIPLH | 24, |
| SEQ ID | | ARTLAETGRYGAVLGTAFVVDGGIYDHEFV | 28, 36, 124, |
| NO: 33 | | ASAVIDGMMNVQLDTGVPVLSAVLTPHEYE | 125, 127, |
| | | DSDADTLLFLALFAVKGMEAARACVEILAA | 128, 129, 131, |
| | | REKIAA | 132, 133, |
| | | | 135, 139 |
| I53- | trimer | NQHSHKDHETVRIAVVRARWHAEIVDACVS | I53-50B: |
| 50B.4PosT1 | | AFEAAMRDIGGDRFAVDVEDVPGAYEIPLH | 24, |
| SEQ ID | | ARTLAETGRYGAVLGTAFVVNGGIYRHEFV | 28, 36, 124, |
| NO: 34 | | ASAVINGMMNVQLNTGVPVLSAVLTPHNYD | 125, 127, |
| | | KSKAHTLLFLALFAVKGMEAARACVEILAA | 128, 129, 131, |
| | | REKIAA | 132, 133, |
| | | | 135, 139 |
| I53-40A | pentamer | TKKVGIVDTTFARVDMASAAILTLKMESPN | |
| genus | | IKIIRKTVPGIKDLPVACKKLLEEEGCDIV | |
| SEQ ID | | MALGMPGK(A/K)EKDKVCAHEASLGLMLA | |
| NO: 35 | | QLMTNKHIIEVFVHEDEAKDDAELKILAAR | |
| | | RAIEHALNVYYLLFKPEYLTRMAGKGLRQG | |
| | | FEDAGPARE | |
| I53-40B | trimer | (S/D)(T/D)INNQLK(A/R)LKVIPVIAI | |
| genus | | DNAEDIIPLGKVLAENGLPAAEITFRSSAA | |
| SEQ ID | | VKAIMLLRSAQPEMLIGAGTILNGVQALAA | |
| NO: 36 | | KEAGA(T/D)FVVSPGFNPNTVRACQIIGI | |
| | | DIVPGVNNPSTVE(A/Q)ALEMGLTTLKFF | |
| | | PAEASGGISMVKSLVGPYGDIRLMPTGGIT | |
| | | P(S/D)NIDNYLAIPQVLACGGTWMVDKKL | |
| | | V(T/R)NGEWDEIARLTREIVEQVNP | |
| I53-47A | trimer | PIFTLNTNIKA(T/D)DVPSDFLSLTSRLV | |
| genus | | GLILS(K/E)PGSYVAVHINTDQQLSFGGS | |
| SEQ ID | | TNPAAFGTLMSIGGIEP(S/D)KN(R/E)D | |
| NO: 37 | | HSAVLEDHLNAMLGIPKNRMYIHFV(N/D) | |
| | | L(N/D)GDDVGWNGTTF | |
| I53-47B | pentamer | NQHSHKD(Y/H)ETVRIAVVRARWHADIVD | |
| genus | | ACVEAFEIAMAAIGGDRFAVDVFDVPGAYE | |
| SEQ ID | | IPLHARTLAETGRYGAVLGTAFVV(N/D)G | |
| NO: 38 | | GIY(R/D)HEFVASAVIDGMMNVQL(S/D) | |
| | | TGVPVLSAVLTPH(R/E)Y(R/E)DS(A/D | |
| | | )E(H/D)H(R/E)FFAAHFAVKGVEAARAC | |
| | | IEIL(A/N)AREKIAA | |
| I53-50A | trimer | EELFKKHKIVAVLRANSVEEAIEKAVAVFA | |
| genus | | GGVHLIEITFTVPDADTVIKALSVLKEKGA | |
| SEQ ID | | IIGAGTVTSVEQCRKAVESGAEFIVSPHLD | |
| NO: 39 | | EEISQFCKEKGVFYMPGVMTPTELVKAMKL | |
| | | GH(T/D)ILKLFPGEVVGP(Q/E)FV(K/E | |
| | | )AMKGPFPNVKFVPTGGV(N/D)LD(N/D) | |
| | | VC(E/K)WF(K/D)AGVLAVGVG(S/K/D) | |
| | | ALV(K/E)G(T/D/K)PDEVRE(K/D)AK( | |
| | | A/E/K)FV(E/K)(K/E)IRGCTE | |
| I53-50B | pentamer | NQHSHKD(Y/H)ETVRIAVVRARWHAEIVD | |
| genus | | ACVSAFEAAM(A/R)DIGGDRFAVDVFDVP | |
| SEQ ID | | GAYEIPLHARTLAETGRYGAVLGTAFVV(N | |
| NO: 40 | | /D)GGIY(R/D)HEEVASAVI(D/N)GMMN | |
| | | VQL(S/D/N)TGVPVLSAVLTPH(R/E/N) | |
| | | Y(R/D/E)(D/K)S(D/K)A(H/D)TLLFL | |
| | | ALFAVKGMEAARACVEILAAREKIAA | |
| T32-28A | dimer | GEVPIGDPKELNGMEIAAVYLQPIEMEPRG | |
| SEQ ID | | IDLAASLADIHLEADIHALKNNPNGFPEGF | |
| NO: 41 | | WMPYLTIAYALANADTGAIKTGTLMPMVAD | |
| | | DGPHYGANIAMEKDKKGGFGVGTYALTFLI | |
| | | SNPEKQGFGRHVDEETGVGKWFEPFVVTYF | |
| | | FKYTGTPK | |
| T32-28B | trimer | SQAIGILELTSIAKGMELGDAMLKSANVDL | |
| SEQ ID | | LVSKTISPGKFLLMLGGDIGAIQQAIETGT | |
| NO: 42 | | SQAGEMLVDSLVLANIHPSVLPAISGLNSV | |
| | | DKRQAVGIVETWSVAACISAADLAVKGSNV | |
| | | TLVRVHMAFGIGGKCYMVVAGDVLDVAAAV | |
| | | ATASLAAGAKGLLVYASIIPRPHEAMWRQM | |
| | | VEG | |
| T33-09A | trimer | EEVVLITVPSALVAVKIAHALVEERLAACV | |
| SEQ ID | | NIVPGLTSIYRWQGSVVSDHELLLLVKTTT | |
| NO: 43 | | HAFPKLKERVKALHPYTVPEIVALPIAEGN | |
| | | REYLDWLRENTG | |
| T33-09B | trimer | VRGIRGAITVEEDTPAAILAATIELLLKML | |
| SEQ ID | | EANGIQSYEELAAVIFTVTEDLTSAFPAEA | |
| NO: 44 | | ARLIGMHRVPLLSAREVPVPGSLPRVIRVL | |
| | | ALWNTDTPQDRVRHVYLNEAVRLRPDLESA | |
| | | Q | |
| T33-15A | trimer | SKAKIGIVTVSDRASAGITADISGKAIILA | |
| SEQ ID | | LNLYLTSEWEPIYQVIPDEQDVIETTLIKM | |
| NO: 45 | | ADEQDCCLIVTTGGTGPAKRDVTPEATEAV | |
| | | CDRMMPGFGELMRAESLKEVPTAILSRQTA | |
| | | GLRGDSLIVNLPGDPASISDCLLAVFPAIP | |
| | | YCIDLMEGPYLECNEAMIKPFRPKAK | |
| T33-15B | trimer | VRGIRGAITVNSDTPTSIIIATILLLEKML | |
| SEQ ID | | EANGIQSYEELAAVIFTVTEDLTSAFPAEA | |
| NO: 46 | | ARQIGMHRVPLLSAREVPVPGSLPRVIRVL | |
| | | ALWNTDTPQDRVRHVYLSEAVRLRPDLESA | |
| | | Q | |
| T33-21A | trimer | RITTKVGDKGSTRLFGGEEVWKDSPIIEAN | |
| SEQ ID | | GTLDELTSFIGEAKHYVDEEMKGILEEIQN | |
| NO: 47 | | DIYKIMGEIGSKGKIEGISEERIAWLLKLI | |
| | | LRYMEMVNLKSFVLPGGTLESAKLDVCRTI | |
| | | ARRALRKVLTVTREFGIGAEAAAYLLALSD | |
| | | LLFLLARVIEIEKNKLKEVRS | |
| T33-21B | trimer | PHLVIEATANLRLETSPGELLEQANKALFA | |
| SEQ ID | | SGQFGEADIKSRFVTLEAYRQGTAAVERAY | |
| NO: 48 | | LHACLSILDGRDIATRTLLGASLCAVLAEA | |
| | | VAGGGEEGVQVSVEVREMERLSYAKRVVAR | |
| | | QR | |
| T33-28A | trimer | ESVNTSFLSPSLVTIRDFDNGQFAVLRIGR | |
| SEQ ID | | TGFPADKGDIDLCLDKMIGVRAAQIFLGDD | |
| NO: 49 | | TEDGFKGPHIRIRCVDIDDKHTYNAMVYVD | |
| | | LIVGTGASEVERETAEEEAKLALRVALQVD | |
| | | IADEHSCVTQFEMKLREELLSSDSFHPDKD | |
| | | EYYKDFL | |
| T33-28B | trimer | PVIQTFVSTPLDHHKRLLLAIIYRIVTRVV | |
| SEQ ID | | LGKPEDLVMMTFHDSTPMHFFGSTDPVACV | |
| NO: 50 | | RVEALGGYGPSEPEKVTSIVTAAITAVCGI | |
| | | VADRIFVLYFSPLHCGWNGTNF | |
| T33-31A | trimer | EEVVLITVPSALVAVKIAHALVEERLAACV | |
| SEQ ID | | NIVPGLTSTYREEGSVVSDHELLLLVKTTT | |
| NO: 51 | | DAFPKLKERVKELHPYEVPEIVALPIAEGN | |
| | | REYLDWLRENTG | |

TABLE 2

| | | | | percent |
|---|---|---|---|---|
| | | Oligomer | isoelectric | hydrophobic |
| name | MW | MW | point | (“ILVMFW”) |
| I53-34A | 21427 | 64281 | 5.82 | 0.33 |
| I53-34B | 17083 | 85414 | 6.1 | 0.31 |
| I53-40A | 17091 | 85455 | 6.85 | 0.32 |
| I53-40B | 21789 | 65367 | 4.91 | 0.34 |
| I53-47A | 12191 | 36572 | 6.47 | 0.34 |
| I53-47B | 16956 | 84781 | 6.31 | 0.31 |
| I53-50A | 21783 | 65350 | 6.91 | 0.35 |
| I53-50B | 16839 | 84195 | 5.95 | 0.32 |
| I53-51A | 19967 | 59900 | 8.74 | 0.34 |
| I53-51B | 17178 | 85892 | 6.31 | 0.3 |
| I52-03A | 21026 | 105129 | 6.16 | 0.31 |
| I52-03B | 25875 | 51749 | 5.32 | 0.3 |
| I52-32A | 15015 | 30029 | 5.43 | 0.42 |
| I52-32B | 16877 | 84383 | 6.58 | 0.35 |
| I52-33A | 17914 | 89569 | 7.17 | 0.36 |
| I52-33B | 19215 | 38430 | 7.12 | 0.37 |
| I32-06A | 21632 | 43263 | 6.78 | 0.3 |
| I32-06B | 14736 | 44208 | 6.48 | 0.34 |
| I32-19A | 25405 | 76214 | 7.86 | 0.3 |
| I32-19B | 17186 | 34373 | 6.57 | 0.24 |
| I32-28A | 16648 | 49944 | 5.23 | 0.32 |
| I32-28B | 16173 | 32347 | 4.76 | 0.31 |
| I53-40A.1 | 17148 | 85740 | 7.66 | 0.32 |
| I53-40B.1 | 22070 | 66211 | 4.74 | 0.34 |
| I53-47A.1 | 12233 | 36698 | 5.65 | 0.34 |
| I53-47A.1NegT2 | 12209 | 36626 | 4.4 | 0.34 |
| I53-47B.1 | 17045 | 85226 | 6.04 | 0.31 |
| I53-47B.1NegT2 | 16902 | 84509 | 4.75 | 0.31 |
| I53-50A.1 | 21765 | 65296 | 6.17 | 0.35 |
| I53-50A.1NegT2 | 21746 | 65237 | 4.62 | 0.35 |
| I53-50A.1PosT1 | 21790 | 65369 | 9.07 | 0.35 |
| I53-50B.1 | 16926 | 84631 | 6.04 | 0.32 |
| I53-50B.1NegT2 | 16810 | 84049 | 4.8 | 0.32 |
| I53-50B.4 PosT1 | 16867 | 50602 | 6.77 | 0.32 |
| I53-40A genus | 17091 | 85455 | 6.85 | 0.32 |
| I53-40B genus | 21789 | 65367 | 4.91 | 0.34 |
| I53-47A genus | 12191 | 36572 | 6.47 | 0.34 |
| I53-47B genus | 16956 | 84781 | 6.31 | 0.31 |
| I53-50A genus | 21652 | 64956 | 7.15 | 0.35 |
| I53-50B genus | 16839 | 84195 | 5.95 | 0.32 |
| T32-28A | 17168 | 34337 | 4.79 | 0.29 |
| T32-28B | 18711 | 56132 | 5.52 | 0.36 |
| T33-09A | 11321 | 33963 | 6.08 | 0.36 |
| T33-09B | 13279 | 39838 | 5.14 | 0.34 |
| T33-15A | 18902 | 56705 | 4.53 | 0.3 |
| T33-15B | 13298 | 39895 | 5.65 | 0.34 |
| T33-21A | 19158 | 57474 | 6.04 | 0.35 |
| T33-21B | 13128 | 39383 | 5.73 | 0.28 |
| T33-28A | 17637 | 52910 | 4.5 | 0.31 |
| T33-28B | 12302 | 36907 | 6.59 | 0.38 |
| T33-31A | 11329 | 33987 | 4.88 | 0.35 |
| T33 dn2A | 13632 | 40896 | 4.7 | 0.19 |
| T33 dn2B | 13687 | 41061 | 5.57 | 0.19 |
| T33 dn5A | 13528 | 40583 | 4.07 | 0.19 |
| T33 dn5A | 19741 | 59222 | 5.45 | 0.29 |
| T33 dn10A | 13883 | 41649 | 4.26 | 0.2 |
| T33 dn10B | 30222 | 90666 | 6.31 | 0.3 |
| I53 dn5A | 17004 | 85019 | 7.14 | 0.35 |
| I53 dn5B | 14138 | 70688 | 4.94 | 0.19 |

Table 1 and 2 provides the amino acid sequence of the compA and compBs of embodiments of the present disclosure. In each case, the pairs of sequences together form an I53 multimer with icosahedral symmetry. The right hand column in Table 1 identifies the residue numbers in each exemplary polypeptide that were identified as present at the interface of resulting assembled virus-like particles (i.e.: “identified interface residues”). As can be seen, the number of interface residues for the exemplary polypeptides of SEQ ID NO:1-34 range from 4-13. In some embodiments, compA and compB have 4, 5, 6, 7, 8, 9, 10, 11, 12, or 13 interface residues. In various embodiments, the compA and compBs comprise an amino acid sequence that is at least 75%, 80%, 85%, 90%, 91%, 92%, 93%, 94%, 95%, 96%, 97%, 98%, or 99% identical over its length, and identical to at least 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOS: 1-34. SEQ ID NOs: 35-51 represent other amino acid sequences of the compA and compBs from embodiments of the present disclosure. In other embodiments, the compA and/or compBs comprise an amino acid sequence that is at least 75%, 80%, 85%, 90%, 91%, 92%, 93%, 94%, 95%, 96%, 97%, 98%, or 99% identical over its length, and identical at least at 20%, 25%, 33%, 40%, 50%, 60%, 70%, 75%, 80%, 90%, or 100% of the identified interface positions, to the amino acid sequence of a polypeptide selected from the group consisting of SEQ ID NOS:1-51.

As shown in Table 2, the compB proteins have similar molecular weights (MW), isoelectric points (pI) and percent hydrophobic residues, suggesting they can be expressed and purified using similar methods.

As is the case with proteins in general, the polypeptides are expected to tolerate some variation in the designed sequences without disrupting subsequent assembly into virus-like particles: particularly when such variation comprises conservative amino acid substitutions. As used here, “conservative amino acid substitution” means that: hydrophobic amino acids (Ala, Cys, Gly, Pro, Met, Val, Ile, Leu) can only be substituted with other hydrophobic amino acids; hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains; amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

In various embodiments of the pbVLPs of the invention, the compA and compBs, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 95%, at least 99%, at least 100% over its length, and/or identical at least at one identified interface position, to the amino acid sequence indicated by the SEQ ID NO):

SEQ ID NO: 1 and SEQ ID NO: 2
(I53-34A and I53-34B);
SEQ ID NO: 3 and SEQ ID NO: 4
(I53-40A and I53-40B);
SEQ ID NO: 3 and SEQ ID NO: 24
(I53-40A and I53-40B.1);
SEQ ID NO: 23 and SEQ ID NO: 4
(I53-40A.1 and I53-40B);
SEQ ID NO: 35 and SEQ ID NO: 36
(I53-40A genus and I53-40B genus);
SEQ ID NO: 5 and SEQ ID NO: 6
(I53-47A and I53-47B);
SEQ ID NO: 5 and SEQ ID NO: 27
(I53-47A and I53-47B.1);
SEQ ID NO: 5 and SEQ ID NO: 28
(I53-47A and I53-47B.1NegT2);
SEQ ID NO: 25 and SEQ ID NO: 6
(I53-47A.1 and I53-47B);
SEQ ID NO: 25 and SEQ ID NO: 27
(I53-47A.1 and I53-47B.1);
SEQ ID NO: 25 and SEQ ID NO: 28
(I53-47A.1 and I53-47B.1NegT2);
SEQ ID NO: 26 and SEQ ID NO: 6
(I53-47A.1NegT2 and I53-47B);
SEQ ID NO: 26 and SEQ ID NO: 27
(I53-47A.1NegT2 and I53-47B.1);
SEQ ID NO: 26 and SEQ ID NO: 28
(I53-47A.1NegT2 and I53-47B.1NegT2);
SEQ ID NO: 37 and SEQ ID NO: 38
(I53-47A genus and I53-47B genus);
SEQ ID NO: 7 and SEQ ID NO: 8
(I53-50A and I53-50B);
SEQ ID NO: 7 and SEQ ID NO: 32
(I53-50A and I53-50B.1);
SEQ ID NO: 7 and SEQ ID NO: 33
(I53-50A and I53-50B.1NegT2);
SEQ ID NO: 7 and SEQ ID NO: 34
(I53-50A and I53-50B.4PosT1);
SEQ ID NO: 29 and SEQ ID NO: 8
(I53-50A.1 and I53-50B);
SEQ ID NO: 29 and SEQ ID NO: 32
(I53-50A.1 and I53-50B.1);
SEQ ID NO: 29 and SEQ ID NO: 33
(I53-50A.1 and I53-50B.1NegT2);
SEQ ID NO: 29 and SEQ ID NO: 34
(I53-50A.1 and I53-50B.4PosT1);
SEQ ID NO: 30 and SEQ ID NO: 8
(I53-50A.1NegT2 and I53-50B);
SEQ ID NO: 30 and SEQ ID NO: 32
(I53-50A.1NegT2 and I53-50B.1);
SEQ ID NO: 30 and SEQ ID NO: 33
(I53-50A.1NegT2 and I53-50B.1NegT2);
SEQ ID NO: 30 and SEQ ID NO: 34
(I53-50A.1NegT2 and I53-50B.4PosT1);
SEQ ID NO: 31 and SEQ ID NO: 8
(I53-50A.1PosT1 and I53-50B);
SEQ ID NO: 31 and SEQ ID NO: 32
(I53-50A.1PosT1 and I53-50B.1);
SEQ ID NO: 31 and SEQ ID NO: 33
(I53-50A.1PosT1 and I53-50B.1NegT2);
SEQ ID NO: 31 and SEQ ID NO: 34
(I53-50A.1PosT1 and I53-50B.4PosT1);
SEQ ID NO: 39 and SEQ ID NO: 40
(I53-50A genus and I53-50B genus);
SEQ ID NO: 9 and SEQ ID NO: 10
(I53-51A and I53-51B);
SEQ ID NO: 11 and SEQ ID NO: 12
(I52-03A and I52-03B);
SEQ ID NO: 13 and SEQ ID NO: 14
(I52-32A and I52-32B);
SEQ ID NO: 15 and SEQ ID NO: 16
(I52-33A and I52-33B)
SEQ ID NO: 17 and SEQ ID NO: 18
(I32-06A and I32-06B);
SEQ ID NO: 19 and SEQ ID NO: 20
(I32-19A and I32-19B);
SEQ ID NO: 21 and SEQ ID NO: 22
(I32-28A and I32-28B);
SEQ ID NO: 23 and SEQ ID NO: 24
(I53-40A.1 and I53-40B.1);
SEQ ID NO: 41 and SEQ ID NO: 42
(T32-28A and T32-28B);
SEQ ID NO: 43 and SEQ ID NO: 44
(T33-09A and T33-09B);
SEQ ID NO: 45 and SEQ ID NO: 46
(T33-15A and T33-15B);
SEQ ID NO: 47 and SEQ ID NO: 48
(T33-21A and T33-21B);
SEQ ID NO: 49 and SEQ ID NO: 50
(T33-28A and T32-28B);
and
SEQ ID NO: 51 and SEQ ID NO: 44
(T33-31A and T33-09B
(also referred to as T33-31B)).

wherein those ending in “A” are compA and those ending in “B” are compB (e.g. I53-34A is compA and I53-34B is compB).

Non-limiting examples of designed protein complexes useful in protein-based VLPs of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety.

In various embodiments of the pbVLPs of the disclosure, the compA and compBs comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 95%, at least 99%, at least 100% over its length, and/or identical at least at one identified interface position, to the amino acid sequence indicated by the SEQ ID NO):

SEQ ID NO: 52 and SEQ ID NO: 53
(T33_dn2A and T33_dn2B);
SEQ ID NO: 54 and SEQ ID NO: 55
(T33_dn5 A and T33_dn5B);
SEQ ID NO: 56 and SEQ ID NO: 57
(T33_dn10A and T33_dn10B);
or
SEQ ID NO: 58 and SEQ ID NO: 59
(153_dn5A and I53_dn5B),

wherein those ending in “dn5B” are compA and those ending in “dn5A” are compB (e.g. I53_dn5B is compA and dn5A is compB).

T33_dn2A
(SEQ ID NO: 52)
NLAEKMYKAGNAMYRKGQYTIAIIAYTLALLKDPNNAEAW
YNLGNAAYKKGEYDEAIEAYQKALELDPNNAEAWYNLGNA
YYKQGDYDEAIEYYKKALRLDPRNVDAIENLIEAEEKQG
T33_dn2B
(SEQ ID NO: 53)
EEAELAYLLGELAYKLGEYRIAIRAYRIALKRDPNNAEAW
YNLGNAYYKQGDYREAIRYYLRALKLDPENAEAWYNLGNA
LYKQGKYDLAIIAYQAALEEDPNNAEAKQNLGNAKQKQG
T33_dn5A
(SEQ ID NO: 54)
NSAEAMYKMGNAAYKQGDYILAIIAYLLALEKDPNNAEAW
YNLGNAAYKQGDYDEAIEYYQKALELDPNNAEAWYNLGNA
YYKQGDYDEAIEYYEKALELDPNNAEALKNLLEAIAEQD
T33_dn5B
(SEQ ID NO: 55)
TDPLAVILYIAILKAEKSIARAKAAEALGKIGDERAVEPL
IKALKDEDALVRAAAADALGQIGDERAVEPLIKALKDEEG
LVRASAAIALGQIGDERAVQPLIKALTDERDLVRVAAAVA
LGRIGDEKAVRPLIIVLKDEEGEVREAAAIALGSIGGERV
RAAMEKLAERGTGFARKVAVNYLETHK
T33_dn10A
(SEQ ID NO: 56)
EEAELAYLLGELAYKLGEYRIAIRAYRIALKRDPNNAEAW
YNLGNAYYKQGDYDEAIEYYQKALELDPNNAEAWYNLGNA
YYKQGDYDEAIEYYEKALELDPENLEALQNLLNAMDKQG
T33_dn10B
(SEQ ID NO: 57)
IEEVVAEMIDILAESSKKSIEELARAADNKTTEKAVAEAI
EEIARLATAAIQLIEALAKNLASEEFMARAISAIAELAKK
AIEAIYRLADNHTTDTFMARAIAAIANLAVTAILAIAALA
SNHTTEEFMARAISAIAELAKKAIEAIYRLADNHTTDKFM
AAAIEAIALLATLAILAIALLASNHTTEKFMARAIMAIAI
LAAKAIEAIYRLADNHTSPTYIEKAIEAIEKIARKAIKAI
EMLAKNITTEEYKEKAKKIIDIIRKLAKMAIKKLEDNRT
I53_dn5A
(SEQ ID NO: 58)
KYDGSKLRIGILHARWNAEIILALVLGALKRLQEFGVKRE
NIIIETVPGSFELPYGSKLFVEKQKRLGKPLDAIIPIGVL
IKGSTMHFEYICDSTTHQLMKLNFELGIPVIFGVLTCLTD
EQAEARAGLIEGKMHNHGEDWGAAAVEMATKFN
I53_dn5B
(SEQ ID NO: 59)
EEAELAYLLGELAYKLGEYRIAIRAYRIALKRDPNNAEAW
YNLGNAYYKQGRYREAIEYYQKALELDPNNAEAWYNLGNA
YYERGEYEEAIEYYRKALRLDPNNADAMQNLLNAKMREE

### Expression and Purification Methods

Overexpression of heterologous proteins in E. coli frequently leads aggregation and deposition in dense, insoluble particles, also known as inclusion bodies. While expression in inclusion bodies may increase the purity of the resulting recombinant protein, it is widely known that to preserve the desired tertiary structure of a protein it is usually preferable to purify the protein from the soluble fraction of the cell. When proteins are expressed in inclusion bodies, denaturation and refolding of the protein is considered crucial. For this reason, solubilization usually is carried out in high concentrations of chaotropic agents like urea or guanidinium hydrochloride to reach complete unfolding. Reducing agents such as 2-mercaptoethanol (β-ME), dithiothreitol (DTT) or 1-monothioglycerol (MTG) are added to reduce non-native inter- and intramolecular disulfide bonds and keep the cysteines in a reduced state.

In contrast, the presently disclosed methods use low concentrations of chaotropic agents to release compB proteins from inclusion bodies without denaturing them. Thus, provided herein is a method of making a nanostructure, comprising solubilizing a recombinant component B (compB) protein from inclusion bodies with a solubilization solution, thereby generating a product sample comprising product compB protein. In some embodiments, the method does not comprise denaturing the compB protein and/or does not comprises refolding the compB protein.

Inclusion bodies may be generated using various recombinant expression systems. E. coli or other bacterial expression systems may be used. In some embodiments, the E. coli is strain K-12. In some embodiments, the E. coli is strain B. In some embodiments, the E. coli is strain W3110 ompT.

The present inventors have determined that both T7 and phoA-based expression systems are able to generate suitable yields of compB protein. However, the phoA-based expression system, in some cases, generated compB protein in greater yield. Suitable expression techniques are provided in the references cited herein, which are incorporated by reference. In some embodiments, the expression is performed at low temperatures, as the inventors have found that expression at about 30° C. generated compB protein in inclusion bodies. In some embodiments, the bacterial cell is cultured at less than about 33° C., optionally at about 15° C. to about 33° C. or at about 17° C. to about 30° C., preferably at about 30° C. In some embodiments, the bacterial cell is cultured at about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., or about 33° C.

The host (e.g., bacterial cell) comprises a polynucleotide encoding the compB protein, the polynucleotide operatively linked to a promoter. The promoter may be a phoA promoter or any other promoter suitable for recombinant protein expression. In some embodiments, the promoter is a phoA promoter. In some embodiments, the promoter is a T7 promoter. In some embodiments, the promoter is a promoter other than a T7 promoter.

Inclusion bodies may be harvested using any technique known in the art, including without limitation chemical and/or physical lysis of the host cell. In some embodiments, the method comprises lysing the bacterial cell in a lysis solution, wherein the lysis solution is substantially free of agents that promote solubility of inclusion bodies; and recovering the inclusion bodies. In some embodiments, the lysis solution is substantially free of detergents. Inclusion bodies may be purified from the cytoplasm of the host cell by centrifugation and/or filtration.

After inclusion bodies are harvested, the compB protein is solubilized using a solubilization solution. Solubilization may be promoted by stirring or otherwise mixing the solution, e.g. by vortexing the solution or sonicating the solution. Various solubilization solutions may be used, including solutions comprising urea, or guanidinium hydrochloride. In some embodiments, the solubilization solution comprises urea, optionally at a urea concentration of 0.05 M to 3 M. The urea concentration may be 0.05 M, 0.1 M, 0.2 M, 0.3 M, 0.4 M, 0.5 M, 0.6 M, 0.7 M, 0.8 M, 0.9 M, 1.0 M, 1.1 M, 1.2 M, 1.3 M, 1.4 M, 1.5 M, 1.6 M, 1.7 M, 1.8 M, 1.9 M, 2.0 M, 2.1 M, 2.2 M, 2.3 M, 2.4 M, 2.5 M, 2.6 M, 2.7 M, 2.8 M, 2.9 M, or 3.0 M. In some cases, the solubilization solution comprises a buffer and/or a mild detergent, which may optionally be the same or different excipients. Suitable buffers include Tris buffer, a histidine buffer, a phosphate buffer, a citrate buffer acetate buffer, 3-[(3-cholamidopropyl)dimethylammonio]-1-propanesulfonate (CHAPS) buffer, or combinations thereof, such as phosphate-citrate buffer. CHAPS has the advantage of also acting as a surfactant.

The solubilization solution may be a buffered solution at a predetermined pH, such as a neutral pH. The optimal pH for the solubilization solution may be determined by testing the procedure at various pH's to determine an optimal value, e.g., a pH of 5-6, 6-7, 7-8 or 8-9. A suitable pH for many compB proteins is pH 7.4. Prior to the solubilization step, the inclusion bodies may optionally be washed. In some embodiments, prior to the solubilization step, the inclusion bodies are washed. Various wash solutions may be used. In some cases, the wash solution comprises a chaotropic agent at a lower concentration than the solubilization solution, e.g., a urea concentration of less than 150 mM, optionally 50-150 mM. In some embodiments, the chaotropic agent is selected from urea, guanidinium hydrochloride, and n-propanol. In some embodiments, the chaotropic agent is urea. In some embodiments, the urea concentration is less than 150 mM, less than 140 mM, less than 130 mM, less than 120 mM, less than 110 mM, less than 100 mM, less than 90 mM, less than 80 mM, less than 70 mM, less than 60 mM, or less than 50 mM. In some embodiments, the chaotropic agent is guanidinium hydrochloride. In some embodiments, the guanidinium hydrochloride concentration is less than 3M. In some embodiments, the guanidinium hydrochloride is less than 3M, less than 2.5M, less than 2M, less than 1.5M, less than 1M, less than 0.5M, or less than 0.1M. In some embodiments, the chaotropic agent is n-propanol. In some embodiments, the concentration of n-propanol is no more than 5%. In some embodiments, the concentration of n-propanol is less than 5%. In some embodiments, the concentration of n-propanol is no more than 5%. In some embodiments, the concentration of n-propanol is less than 5%. Once solubilized, the compB protein may be purified further using a variety of biochemical techniques. Advantageously, a purification procedure that removes host cell proteins (HCP), endotoxin, and/or other impurities is employed. Particularly suitable for purification of compB proteins are so-called orthogonal purification strategies. As a non-limited example, ion exchange chromatography may be combined with a mixed-mode chromatography step.

In some embodiments, the method comprises contacting the compB protein with an anion exchange resin, optionally a weak anion exchange resin, optionally a diethylaminoethyl(DEAE)-conjugated resin; and eluting the compB protein from the resin using an elution solution. In some embodiments, the method comprises contacting the compB protein with an anion exchange resin. In some embodiments, the anion exchange resin is a weak anion exchange resin. In some embodiments, the anion exchange resin is a diethylaminoethyl(DEAE)-conjugated resin. In some embodiments, the anion exchange resin is a quaternary amine (Q) strong anion exchange resin. In some embodiments, the method comprises purifying the compB protein using anion-exchange chromatography (see e.g. Sartobind®).

In some embodiments, the method comprises, before the eluting step, washing the anion exchange resin with a column-wash solution. Various column-wash solutions may be used. Disclosed herein are column-wash solutions comprising a zwitterionic surfactant and/or a nonionic surfactant. The nonionic surfactant may be Triton X-100 or an equivalent thereof. The zwitterionic surfactant may be 3-[(3-cholamidopropyl)dimethylammonio]-1-propanesulfonate (CHAPS) or an equivalent thereof. In some embodiments, the zwitterionic surfactant is selected from CHAP SO (3-(3-Cholamidopropyl)dimethylammonio)-2-hydroxy-1 -propanesulfonate), LDAO, DDMAB, and any Zwittergent® surfactant.

Elution from the anion exchange resin may be achieved using a salt gradient. Either a stepwise or a continuous gradient may be used. In some embodiments, the elution solution comprises sodium chloride (NaCl) at a NaCl concentration of 50 mM to 800 mM, e.g., 100 mM, 200 mM, 250 mM, 300 mM, 400 mM, 500 mM, 600 mM, 700 mM, or 800 mM.

In some embodiments, the method comprises purifying the compB protein with a mixed-mode resin. Suitable mixed-mode media includes a ceramic hydroxyapatite (CHT) resin. Hydroxyapatite is a form of calcium phosphate that has long been used in the chromatographic separation of proteins and DNA. (Schröder et al., Analytical Biochemistry 313 (2003) 176-178.) The adsorption of proteins to hydroxyapatite involves both anionic and cationic exchange. Proteins are most commonly adsorbed in a low concentration (10-25 mM) of phosphate buffer, although some acidic proteins are adsorbed only if loaded in water, saline, or a non-phosphate buffer. Proteins are usually eluted by an increasing phosphate gradient, although gradients of Ca²⁺⁺, Mg²⁺⁺, or Cl⁻ or ions are also useful, especially for the selective elution of basic proteins. When using phosphate, acidic proteins are more readily eluted than basic proteins, although the phosphate concentration required to elute any protein can be reduced by raising the pH. A mixture of proteins bound to hydroxyapatite can be fractionated by a series of phosphate wash steps of increasing pH.

In some embodiments, the product compB protein has at least 50% solubility, optionally 70-95% solubility. In some embodiments, the product compB protein has at least 50%, at least 55%, at least 60%, at least 65%, at least 70%, at least 75%, at least 80%, at least 85%, at least 90%, or at least 95% solubility. In some embodiments, the product compB protein has at least 70-95% solubility. In some embodiments, solubility is measured by gel filtration chromatography, optionally using a Superose 6 column.

In some embodiments, the product compB protein has at least 80% purity calculated as weight by weight of total protein (w/w), optionally at least 95% w/w purity. In some embodiments, the product compB protein has at least 80%, at least 85%, at least 90%, or at least 95% w/w purity. In some embodiments, purity is measured by poly-acrylamide gel electrophoresis, optionally denaturing SDS-PAGE.

In some embodiments, the product compB protein is at least 70% w/w assembly competent, optionally 90-98% w/w assembly competent. In some embodiments, the product compB protein is at least 70%, at least 75%, at least 80%, at least 85%, at least 90%, at least 95%, or at least 98% w/w assembly competent. In some embodiments, the product compB protein is at least 70% w/w assembly competent, optionally 90-100% w/w assembly competent. In some embodiments, percentage of assembly competent compB protein is defined as the percentage of compB protein in the product solution, weight by weight (w/w), that assembles into a protein-based Virus-Like Particle (vpVLP) when the compB protein is mixed with a solution comprising component A (compA) protein in excess. In some embodiments, percentage of assembly competent compB protein is defined as the percentage of compB protein in the product solution, weight by weight (w/w), that assembles into a protein-based Virus-Like Particle (vpVLP) when the compB protein is mixed with a solution comprising component A (compA) protein 10% in excess.

In some embodiments, the product solution comprises less than 50 endotoxin units per milligram of total protein (EU/mg), optionally 5-15 units of EU/mg. In some embodiments, the product solution comprises less than 50, less than 45, less than 40, less than 35, less than 30, less than 25, less than 20, less than 15, less than 10, or 5 units of EU/mg. In some embodiments, the product solution comprises 4, 5, 6, 10, 7, 8, 10, 11, 12, 13, 14, 15 or 16 units of EU/mg.

Further provided herein is a composition comprising compB protein produced by any of the methods of the disclosure. The composition may be, for example, at least 50% soluble, optionally 70-95% soluble; at least 80% pure, wherein purity is calculated as weight by weight of total protein (w/w), optionally at least 95% w/w pure; and/or at least 70% w/w assembly competent, optionally 90-100% w/w assembly competent.

The compB protein may be placed into a final solution using any of various buffer exchange techniques known in the art, including dilution of a concentrated compB solution into a final solution and/or diafiltration/ultrafiltration (DF/UF), e.g., in continuous mode. In some embodiments, the composition comprises one or more of 20 mM tris(hydroxymethyl)aminomethane (Tris) buffer, optionally at 20 mM, and/or 250 mM NaCl, optionally at 250 mM. In some embodiments, the composition is buffered at a pH of 7-8, optionally a pH of 7.4. In some embodiments, the composition is buffered to a pH of 7.0, 7.1, 7.2, 7. 3, 7.4, 7.5, 7.6, 7.7, 7.8, 7.9, or 8.0. In some embodiments, the composition is buffered to a pH of 7.4. In some embodiments, the composition is stable to storage and/or freeze-thaw. In some embodiments, the composition is stable for storage. In some embodiments, the composition is stable for freeze-thaw.

### Exemplary Methods

Provided herein are methods of making a making a nanostructure. In some embodiments, the nanostructure comprises a component B (compB) protein. In some embodiments, the nanostructure comprises a component A (compA) protein. In some embodiments, the nanostructure comprises a compA and a compB protein.

In some embodiments, the method comprises expressing the compB protein in E. coli. In some embodiments, the E. coli is the B-strain. In some embodiments, the E. coli is the K12-strain. In some embodiments, the method comprises isolating a component protein (i.e. compA or compB) from inclusion bodies.

In some embodiments, the method comprises:
(i) isolating inclusion bodies comprising compB proteins from E. coli;
(ii) contacting the inclusion bodies with a solubilization solution comprising a chaotropic agent to solubilize compB proteins, wherein the chaotropic agent is provided at a concentration sufficient to solubilize the compB proteins as monomers capable of assembly into a multimer (e.g., a pentamer);
(iii) contacting compB proteins with an anion exchange resin and eluting the compB protein from the resin using an elution solution;
(iv) purifying the compB protein.

In some embodiments, the method comprises:
(i) isolating inclusion bodies comprising compB proteins from E. coli;
(ii) contacting the inclusion bodies with a wash solution comprising a chaotropic agent at a first concentration, wherein the first concentration is sufficient to purify the inclusion bodies without solubilization of compB;
(iii) contacting the inclusion bodies with a solubilization solution comprising the chaotropic agent at a second concentration to solubilize compB proteins, wherein the concentration is sufficient to solubilize the compB proteins as monomers capable of assembly into a multimer (e.g., a pentamer);
(iv) contacting compB proteins with an anion exchange resin and eluting the compB protein from the resin using an elution solution;
(v) purifying the compB protein. In some embodiments, the method comprises:
(i) isolating inclusion bodies comprising compB proteins from E. coli;
(ii) contacting the inclusion bodies with a solubilization solution comprising a chaotropic agent to solubilize compB proteins, wherein the chaotropic agent is provided at a concentration sufficient to solubilize the compB proteins as monomers capable of assembly into a multimer (e.g., a pentamer);
(iii) contacting compB proteins with an anion exchange resin and eluting the compB protein from the resin using an elution solution;
(iv) purifying the compB protein with a mixed-mode resin.

In some embodiments, the method comprises:
(i) isolating inclusion bodies comprising compB proteins from E. coli;
(ii) contacting the inclusion bodies with a wash solution comprising a chaotropic agent at a first concentration, wherein the first concentration is sufficient to purify the inclusion bodies without solubilization of compB;
(iii) contacting the inclusion bodies with a solubilization solution comprising the chaotropic agent at a second concentration to solubilize compB proteins, wherein the concentration is sufficient to solubilize the compB proteins as monomers capable of assembly into a multimer (e.g., a pentamer);
(iv) contacting compB proteins with an anion exchange resin and eluting the compB protein from the resin using an elution solution;
(v) purifying the compB protein with a mixed-mode resin.

In some embodiments, the method comprises:
(i) isolating inclusion bodies comprising compB proteins from E. coli;
(ii) contacting the inclusion bodies with a solubilization solution comprising urea to solubilize compB proteins, wherein urea is provided at a concentration sufficient to solubilize the compB proteins as monomers capable of assembly into a multimer (e.g., a pentamer);
(iii) contacting compB proteins with an anion exchange resin and eluting the compB protein from the resin using an elution solution;
(iv) purifying the compB protein.

In some embodiments, the method comprises:
(i) isolating inclusion bodies comprising compB proteins from E. coli;
(ii) contacting the inclusion bodies with a wash solution comprising urea at a first concentration, wherein the first concentration is sufficient to purify the inclusion bodies without solubilization of compB;
(iii) contacting the inclusion bodies with a solubilization solution comprising urea at a second concentration to solubilize compB proteins, wherein the concentration is sufficient to solubilize the compB proteins as monomers capable of assembly into a multimer (e.g., a pentamer);
(iv) contacting compB proteins with an anion exchange resin and eluting the compB protein from the resin using an elution solution;
(v) purifying the compB protein.

In some embodiments, the method comprises:
(i) isolating inclusion bodies comprising compB proteins from E. coli;
(ii) contacting the inclusion bodies with a solubilization solution comprising urea to solubilize compB proteins, wherein urea is provided at a concentration sufficient to solubilize the compB proteins as monomers capable of assembly into a multimer (e.g., a pentamer);
(iii) contacting compB proteins with an anion exchange resin and eluting the compB protein from the resin using an elution solution;
(iv) purifying the compB protein with a mixed-mode resin.

In some embodiments, the method comprises:
(i) isolating inclusion bodies comprising compB proteins from E. coli;
(ii) contacting the inclusion bodies with a wash solution comprising urea at a first concentration, wherein the first concentration is sufficient to purify the inclusion bodies without solubilization of compB;
(iii) contacting the inclusion bodies with a solubilization solution comprising urea at a second concentration to solubilize compB proteins, wherein the concentration is sufficient to solubilize the compB proteins as monomers capable of assembly into a multimer (e.g., a pentamer);
(iv) contacting compB proteins with an anion exchange resin and eluting the compB protein from the resin using an elution solution;
(v) purifying the compB protein with a mixed-mode resin.

In some embodiments, the method comprises:
(i) isolating inclusion bodies comprising compB proteins from E. coli;
(ii) contacting the inclusion bodies with a wash solution comprising urea at a concentration of less than 150 mM for a duration of time, wherein the concentration is sufficient to solubilize the compB proteins as monomers capable of assembly into a multimer (e.g., a pentamer);
(iii) contacting the inclusion bodies with a solubilization solution comprising urea at a concentration of 0.15M to 2M for a duration of time to solubilize compB proteins, wherein the concentration is sufficient to solubilize the compB proteins as monomers capable of assembly into a multimer (e.g., a pentamer)
(iv) contacting compB proteins with a diethylaminoethyl(DEAE)-conjugated resin and eluting the compB protein from the resin using an elution solution;
(v) purifying the compB protein with a ceramic hydroxyapatite (CHT) resin.

In some embodiments, the method comprises:
(i) isolating inclusion bodies comprising compB proteins having the sequence set forth in SEQ ID NO: 58 from E. coli;
(ii) contacting the inclusion bodies with a wash solution comprising urea at a concentration of less than 150 mM for a duration of time;
(iii) contacting the inclusion bodies with a solubilization solution comprising urea at a concentration of 0.15M to 2M for a duration of time to solubilize compB proteins, wherein the concentration is sufficient to solubilize the compB proteins as monomers capable of assembly into a multimer (e.g., a pentamer);
(iv) contacting compB proteins with a diethylaminoethyl(DEAE)-conjugated resin and eluting the compB protein from the resin using an elution solution;
(v) purifying the compB protein with a ceramic hydroxyapatite (CHT) resin.

### Assembly of pbVLPs

In some embodiments, a single component self-assembles into the pbVLP. In some embodiments, one or more purified samples of first and second components for use in forming a pbVLP are mixed in an approximately equimolar molar ratio in aqueous conditions (e.g., an I53-50A/B icosahedral pbVLP). The first and second components (through the multimerization domains and optionally through the ectodomains) interact with one another to drive assembly of the target pbVLP. Successful assembly of the target pbVLP can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled pbVLP can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the antigenic protein in the pbVLP can be assessed by techniques commonly used to determine the identity of protein molecules in aqueous solutions, including but not limited to SDS-PAGE, mass spectrometry, protein sequencing, ELISA, surface plasmon resonance, biolayer interferometry, or amino acid analysis. The accessibility of the protein on the exterior of the particle, as well as its conformation or antigenicity, can be assessed by techniques commonly used to detect the presence and conformation of an antigen, including but not limited to binding by monoclonal antibodies, conformation-specific monoclonal antibodies, surface plasmon resonance, biolayer interferometry, or antisera specific to the antigen.

In various embodiments, the pbVLPs of the disclosure comprise two or more distinct compAs bearing different antigenic proteins as genetic fusions; these pbVLPs co-display multiple different proteins on the same pbVLP. These multi-antigen pbVLPs are produced by performing in vitro assembly with mixtures of two or more antigens each comprising a multimerization domain. The fraction of each antigen in the mixture determines the average valency of each antigenic protein in the resulting pbVLPs. The presence and average valency of each antigen in a given sample can be assessed by quantitative analysis using the techniques described above for evaluating the presence of antigenic proteins in full-valency pbVLPs.

In various embodiments, the pbVLPs are between about 20 nanometers (nm) to about 40 nm in diameter, with interior lumens between about 15 nm to about 32 nm across and pore sizes in the protein shells between about 1 nm to about 14 nm in their longest dimensions.

In some embodiments, the pbVLPs has icosahedral symmetry. In such embodiment, the pbVLP may comprise 60 copies of a first component and 60 copies of a second component. In one such embodiment, the number of identical compAs in each first assembly is different than the number of identical compAs in each second assembly. For example, in some embodiments, the pbVLP comprises twelve first assemblies and twenty second assemblies; in such embodiments, each first assembly may, for example, comprise five copies of the identical first component, and each second assembly may, for example, comprise three copies of the identical second component. In other embodiments, the pbVLP comprises twelve first assemblies and thirty second assemblies; in such an embodiment, each first assembly may, for example, comprise five copies of the identical first component, and each second assembly may, for example, comprise two copies of the identical second component. In further embodiments, the pbVLP comprises twenty first assemblies and thirty second assemblies; in this embodiment, each first assembly may, for example, comprise three copies of the identical first component, and each second assembly may, for example, comprise two copies of the identical second component. All of these embodiments are capable of forming protein-based pbVLPs with regular icosahedral symmetry.

In various further embodiments, oligomeric states of the first and second multimerization domains are as follows:
I53-34A: trimer+I53-34B: pentamer;
I53-40A: pentamer+I53-40B: trimer;
I53-47A: trimer+I53-47B: pentamer;
I53-50A: trimer+I53-50B: pentamer;
I53-51A: trimer+I53-51B: pentamer;
I32-06A: dimer+I32-06B: trimer;
I32-19A: trimer+I32-19B: dimer;
I32-28A: trimer+I32-28B: dimer;
I52-03A: pentamer+I52-03B: dimer;
I52-32A: dimer+I52-32B: pentamer; and
I52-33A: pentamer+I52-33B: dimer

### Nucleic Acids

In another aspect, the present disclosure provides isolated nucleic acids encoding an antigen, a first component, and/or a second component, of the present disclosure. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

In a further aspect, the present disclosure provides recombinant expression vectors comprising the isolated nucleic acid of any embodiment or combination of embodiments of the disclosure operatively linked a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred embodiment, the expression vector comprises a plasmid. However, the disclosure is intended to include other expression vectors that serve equivalent functions, such as viral vectors.

In another aspect, the present disclosure provides host cells that have been transfected or transduced with the recombinant expression vectors disclosed herein, wherein the host cells can be either prokaryotic or eukaryotic. The cells can be transiently or stably transfected or transduced. Such transfection or transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, NY).

In another aspect, the disclosure provides a method of producing an antigen, component, or pbVLP according to the disclosure. In some embodiments, the method comprises the steps of (a) culturing a host according to this aspect of the disclosure under conditions conducive to the expression of the polypeptide, and (b) optionally, recovering the expressed polypeptide.

In some embodiments, the disclosure provides a method of manufacturing a vaccine, comprising culturing a host cell comprising a polynucleotide comprising a sequence encoding the antigen of the disclosure in a culture medium so that the host cell secretes the antigen into the culture media; optionally purifying the antigen from the culture media; mixing the antigen with a second component, wherein the second component multimerizes with the antigen to form a pbVLP; and optionally purifying the pbVLP.

In some embodiments, the disclosure provides method of manufacturing a vaccine, comprising culturing a host cell comprising one or more polynucleotides comprising sequences encoding both components of the pbVLP of any one of disclosure so that the host cell secretes the first component and the second component into the culture media; and optionally purifying the pbVLP from the culture media.

Illustrative host cells in include E. coli cells, 293 and 293F cells, HEK293 cells, Sf9 cells, Chinese hamster ovary (CHO) cells and any other cell line used in the production of recombinant proteins.

### Formulation

In some embodiments, the buffer in the composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate- 60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the pbVLP, in lyophilized or liquid form. Exemplary stabilizers include sucrose, sorbitol, glycine, inositol, sodium chloride, methionine, arginine, and arginine hydrochloride.

### Methods of Use

In some embodiments, the disclosure provides methods for treating or preventing a disease or disorder in a subject in need thereof comprising administering a nanostructure (e.g., pbVLP) prepared according to a method described herein. In some embodiments, the disclosure provides a method for inducing, promoting, or increasing an immune response in a subject in need thereof, comprising administering a nanostructure (e.g., pbVLP) prepared according to a method described. In some embodiments, the nanostructure comprises one or more antigens. In some embodiments, the one or more antigens are displayed on the surface of the nanostructure. In some embodiments, the nanostructure comprises one or more immunostimulatory molecules attached to the exterior and/or encapsulated in the cage interior. As used herein, an immunostimulatory molecules is a compound that stimulates an immune response (including enhancing a pre-existing immune response) in a subject to whom it is administered, whether alone or in combination with another agent (e.g., an antigen). Exemplary immunostimulatory molecules include, but are not limited to, TLR ligands.

In some embodiments, the disclosure provides a method for inducing, promoting, or increasing an immune response in a subject in need thereof, comprising administering a nanostructure (e.g., pbVLP) prepared according to a method described herein as an immunogenic composition or vaccine. Upon introduction into a host, the immunogenic composition or vaccine provokes an immune response. The “immune response” refers to a response that induces, increases, or perpetuates the activation or efficiency of innate or adaptive immunity. In some embodiments, the immune response comprises production of antibodies and/or cytokines. In some embodiments, the immune response comprises activation of cytotoxic T cells, antigen presenting cells, helper T cells, dendritic cells, B cells, and/or other cellular responses.

In some embodiments, the disclosure provides a method for inducing, promoting, or increasing an antibody response in a subject in need thereof, comprising administering a nanostructure (e.g., pbVLP) prepared according to a method described herein as an immunogenic composition or vaccine, wherein the nanostructure displays one or more antigens, and wherein the antibody response is directed to epitopes present on the one or more antigens. Methods for analyzing an antibody response in a subject are known to those of skill in the art. For example, in some embodiments, an increase in an immune response is measured by ELISA to determine antigen-specific antibody titers.

In some embodiments, the disclosure provides a method for inducing, promoting, or increasing an immune response comprising an improved B-memory cell response in a subject in need thereof, comprising administering a nanostructure (e.g., pbVLP) prepared according to a method described herein as an immunogenic composition or vaccine in immunized subjects. An improved B-memory cell response is intended to mean an increased frequency of peripheral blood B cells capable of differentiation into antibody-secreting plasma cells upon antigen encounter as measured by stimulation of in vitro differentiation. In some embodiments, the disclosure provides methods for increasing the number of antibody-secreting B cells. In some embodiments, the antibody-secreting B cells are bone marrow plasma cells or germinal B cells. In some embodiments, methods for measuring antibody secreting B cells includes, but is not limited to, antigen-specific ELISPOT assays and flow cytometry of plasma cells or germinal center B cells collected at various time points post-immunization.

In some embodiments, the nanostructure (e.g., pbVLP) is administered as part of a prophylactic immunogenic composition or vaccine, wherein the immunogenic composition or vaccine confers resistance in a subject to subsequent exposure to infectious agents. In some embodiments, the nanostructure (e.g., pbVLP) is administered as part of a therapeutic immunogenic composition or vaccine, wherein the immunogenic composition or vaccine initiates or enhances a subject's immune response to a pre-existing antigen. In some embodiments, the pre-existing antigen is a viral antigen in a subject infected with an infectious agent or neoplasm. In some embodiments, the pre-existing antigen is a cancer antigen in a subject with a tumor or malignancy. The desired outcome of a prophylactic or therapeutic immune response depends upon the disease or condition being treated, according to principles well known in the art. For example, in some embodiments, an immune response against an infectious agent may completely prevent colonization and replication of an infectious agent. In some embodiments, a vaccine against infectious agents is considered effective if it reduces the number, severity, or duration of symptoms, if it reduces the number of individuals in a population with symptoms, or reduces the transmission of an infectious agent. In some embodiments, an immune response against cancer, allergens, or infectious agents is effective it completely treats a disease, alleviates symptoms, or contributes to an overall therapeutic intervention against a disease.

In some embodiments, the disclosure provides a method for treating or preventing an acute or chronic infectious disease in a subject in need thereof, comprising administering a nanostructure (e.g., pbVLP) prepared according to a method describe herein as an immunogenic composition or vaccine. In some embodiments, the nanostructure (e.g., pbVLP) comprises one or more infectious disease antigens. In some embodiments, the one or more infectious disease antigens is a microbial antigen. Microbial antigens are antigens derived from a microbial species, e.g., a bacteria, virus, fungus, parasite, or mycobacterium. In some embodiments, the disclosure provides a method for treating or preventing a viral infection in a subject in need thereof, comprising administering a nanostructure (e.g., pbVLP) prepared according to a method describe herein as an immunogenic composition or vaccine, wherein the nanostructure comprises one or more infectious disease antigens derived from the virus. In some embodiments, the viral infection is immunodeficiency (e.g., HIV, papilloma (e.g., HPV), herpes (e.g., HSV), encephalitis, influenza (e.g., human influenza virus A), COVID-19 (e.g., SARS-CoV-2), and common cold (e.g., human rhinovirus, respiratory syncytial virus). In some embodiments, the disclosure provides a method for reducing a viral infection in a subject in need thereof, comprising administering to the subject a nanostructure (e.g., pbVLP) prepared according to a method described herein.

In some embodiments, the disclosure provides a method for treating or preventing a disorder associated with abnormal apoptosis, a differentiation process (e.g., cellular proliferative disorders, e.g., hyperproliferative disorders), or a cellular differentiation disorder (e.g., cancer). Examples of cellular proliferative and/or differentiative disorders include cancer (e.g., carcinoma, carcinoma, metastatic disorders, or hematopoietic neoplastic disorders). In some embodiments, an immunogenic composition or vaccine comprising a nanostructure (e.g., pbVLP) prepared according to a method described herein is administered to a subject who has cancer. The term “cancer” refer to malignancies of the various organ systems, including those affecting the lung, breast, thyroid, lymphoid tissues, gastrointestinal organs, and the genitourinary tract, as well as to adenocarcinomas that are generally considered to include malignancies such as most colon cancers, renal-cell carcinomas, prostate cancer and/or testicular tumors, non-small cell carcinoma of the lung, cancer of the small intestine, and cancer of the esophagus. In some embodiments, the immunogenic composition or vaccine is used to treat a subject who has, who is suspected of having, or who may be at high risk for developing any type of cancer.

In some embodiments, the disclosure provides methods for inducing an anti-tumor immune response in a subject with cancer, comprising administering an immunogenic composition or vaccine comprising a nanostructure (e.g., pbVLP) prepared according to a method described herein. In some embodiments, the nanostructure comprises one or more antigens, wherein the antigens are cancer antigens. A cancer antigen is an antigen that is expressed preferably on cancer cells (i.e., it is expressed at higher levels in cancer cells than on non-cancer cells) and in some instances is solely expressed by cancer cells. The cancer antigen may be expressed within a cancer cell or on the surface of the cancer cell. In some embodiments, administering the immunogenic composition or vaccine comprising a nanostructure (e.g., pbVLP) comprising one or more cancer antigens induces an anti-tumor immune response, thereby preventing or treating a cancer in the subject.

### EXAMPLES

### Example 1: Development of E. coli Strains for Production of CompB-01

This example demonstrates expression of compB-01 from the insoluble fraction of E. coli. A combinatorial set of strains were evaluated in both B and K-12 E. coli host backgrounds. Two different promoters, T7 and phoA, were used to evaluate different expression kinetics. Using an optimized DNA coding sequence, a total of ten plasmids were transformed into appropriate hosts to create 27 unique strains (Table 3). Expression evaluation was performed in 3 mL total volume using a 24-well dish. Soluble and insoluble fractions were assayed by SDS-PAGE. Nine strains yielded some detectable product, mostly in the insoluble fraction of the cells. Four strains yielded product that migrated at a higher molecular weight than the reference standard. Eight strains were re-evaluated in shake flasks and expression was compared to a null host. Edman degradation confirmed the correctly processed N-terminus is present in ICXB01, ICXB10 and IXCB11, which express compB-01 as an inclusion body in the cytoplasm. These three were chosen for evaluation in platform, fed-batch fermentation processes in 4×5 L fermentations. Biomass yields of ICXB10 and ICXB11 in the unoptimized process were 170-190 g/L wet cell weight (WCW) and ˜1 g/L insoluble compB-01. Material from ICXB10 was purified, QC tested and evaluated for nanoparticle assembly.

The full sequence of compB-01 is SEQ ID NO: 60;

MNQHSHKDHETVRIAVVRARWHAEIVDACVSAFEAAMRDI
GGDRFAVDVEDVPGAYEIPLHARTLAETGRYGAVLGTAFV
VNGGIYRHEFVASAVINGMMNVQLNTGVPVLSAVLTPHNY
DKSKAHTLLFLALFAVKGMEAARACVEILAAREKIAA

The full list of tested constructs is provided in Table 3.

TABLE 3

| | | | | | Compart- | Host | |
|---|---|---|---|---|---|---|---|
| Strain # | Plasmid # | Promoter^{a} | Leader | Relevant Host Genotype | ment | Source | Host ID |
| ICXB01 | pICXB1 | T7 | None | BL21 T7 LysY I^{Q} | cyto | NEB | C3013 |
| ICXB02 | pICXB2 | T7 | PhoA | BL21 T7 LysY I^{Q} | ppl | NEB | C3013 |
| ICXB03 | pICXB2 | T7 | PhoA | BL21 T7 LysY I^{Q} degP | ppl | NEB | CBM126 |
| ICXB04 | pICXB3 | T7 | STII(v3) | BL21 T7 LysY I^{Q} | ppl | NEB | C3013 |
| ICXB05 | pICXB3 | T7 | STII(v3) | BL21 T7 LysY I^{Q} degP | ppl | NEB | CBM126 |
| ICXB06 | pICXB4 | T7 | STII(v5) | BL21 T7 LysY I^{Q} | ppl | NEB | C3013 |
| ICXB07 | pICXB4 | T7 | STII(v5) | BL21 T7 LysY I^{Q} degP | ppl | NEB | CBM126 |
| ICXB08 | pICXB5 | T7 | STII(v7) | BL21 T7 LysY I^{Q} | ppl | NEB | C3013 |
| ICXB09 | pICXB5 | T7 | STII(V7) | BL21 T7 LysY I^{Q} degP | ppl | NEB | CBM126 |
| ICXB10 | pICXB6 | phoA | None | W3110 ompT | cyto | CYT | CBM179 |
| ICXB11 | pICXB6 | phoA | None | BL21 | cyto | NEB | C2530 |
| ICXB12 | pICXB7 | phoA | PhoA | BL21 | ppl | NEB | C2530 |
| ICXB13 | pICXB7 | phoA | PhoA | BL21 degP ptrA | ppl | CYT | CBM185 |
| ICXB14 | pICXB7 | phoA | PhoA | W3110 ompT | ppl | CYT | CBM181 |
| ICXB15 | pICXB7 | phoA | PhoA | W3110 ompT degP ptrA prc | ppl | CYT | CBM163 |
| ICXB16 | pICXB8 | phoA | STII(v3) | BL21 | ppl | NEB | C2530 |
| ICXB17 | pICXB8 | phoA | STII(v3) | BL21 degP ptrA | ppl | CYT | CBM185 |
| ICXB18 | pICXB8 | phoA | STII(v3) | W3110 ompT | ppl | CYT | CBM181 |
| ICXB19 | pICXB8 | phoA | STII(v3) | W3110 ompT degP ptrA prc | ppl | CYT | CBM163 |
| ICXB20 | pICXB9 | phoA | STII(v5) | BL21 | ppl | NEB | C2530 |
| ICXB21 | pICXB9 | phoA | STII(v5) | BL21 degP ptrA | ppl | CYT | CBM185 |
| ICXB22 | pICXB9 | phoA | STII(v5) | W3110 ompT | ppl | CYT | CBM181 |
| ICXB23 | pICXB9 | phoA | STII(v5) | W3110 ompT degP ptrA prc | ppl | CYT | CBM163 |
| ICXB24 | pICXB10 | phoA | STII(v7) | BL21 | ppl | NEB | C2530 |
| ICXB25 | pICXB10 | phoA | STII(v7) | BL21 degP ptrA | ppl | CYT | CBM185 |
| ICXB27 | pICXB10 | phoA | STII(v7) | W3110 ompT | ppl | CYT | CBM181 |
| ICXB28 | pICXB10 | phoA | STII(v7) | W3110 ompT degP ptrA prc | ppl | CYT | CBM163 |

All incubations were performed at 30° C. unless otherwise specified. All broth cultures were incubated shaking at 30° C., 250 rpm, 1″ orbit. After incubation for 2 ±0.5 hr, T7 cultures were induced by the addition of 3 μL of 1 M IPTG to each well. Although the cultures were induced based on time, the anticipated OD600 was 0.6-0.9 at induction. After addition of IPTG, cultures were incubated for an additional 4±0.5 hr. At harvest, the final OD600 of each well was recorded. PhoA strains were prepared similarly to the T7 strains, except the expression medium was Completely Repressed Alkaline Phosphatase, (C.R.A.P). PhoA strains were incubated for 24±1 hr.

Subsequent fed-batch fermentation screening was performed, using the fermentation configuration provided in Table 4, by seeding fermenters at 1% (v/v) and growing the cultures without manipulation for 8-12 hr until the glycerol in the batch medium was exhausted, then the dissolved oxygen (DO) increased sharply (“spike”). After observation of a DO spike, addition of feed was initiated. Feed schedules were designed to ramp exponentially.

TABLE 4
Fermentation Configuration

| Process Parameter | Process | Comments |
|---|---|---|
| Temperature | Control Range | |
| | 30 ± 2° C. | Growth and production ferms A-D Shift |
| | | to 25° C. at induction in fermenter B |
| Agitation | 750 rpm | Fixed |
| Airflow | 4.5 LPM | ptal flow. All gasses sparged through |
| | | microsparger medium. |
| Oxygen supplementation | 0-100 % | Supplemented by control of DO cascade. |
| pH | 6.8 ± 1 | Controlled with acid and base |
| Dissolved oxygen (DO) | 35 ± 10% (T7) | Cascade to oxygen supplementation. |
| | 40 ± 10% (phoA) | |

Around the time of the peak feed rate phosphate becomes limiting in the phoA process which autoinduces the phoA promoter. Therefore, phoA process the induction time was controlled by the total phosphate supplemented in the batch medium. The feed rate was scheduled to drop by 30% per the pre-programmed schedule and remain at a constant rate for the remainder of the fermentation.

At each sampling time, two 1.0 mL whole broth samples were collected and centrifuged for 10 min. The supernatant was transferred to another set of tubes and if necessary the metabolites concentrations were determined, if required. The pellets were weighed to obtain wet cell weight (WCW) and stored frozen for SDS-PAGE analysis. All WCW data are reported as an average of at least two 1.0 mL samples. Material from the harvest was also evaluated for the N-terminal sequence by Edman degradation and particle assembly with compA-01.

The full sequence of compA-01 is SEQ ID NO: 61;

MEELFKKHKIVAVLRANSVEEAIEKAVAVFAGGVHLIEIT
FTVPDADTVIKALSVLKEKGAIIGAGTVTSVEQARKAVES
GAEFIVSPHLDEEISQFAKEKGVFYMPGVMTPTELVKAMK
LGHTILKLFPGEVVGPQFVKAMKGPFPNVKFVPTGGVNLD
NVAEWFKAGVLAVGVGSALVKGTPDEVREKAKAFVEKIRG
ATELE

The fermenters were set to be harvested 12 hr after IPTG addition (T7) or 48 hr after inoculation (phoA). Each fermenter was harvested by bucket centrifugation.

### Results

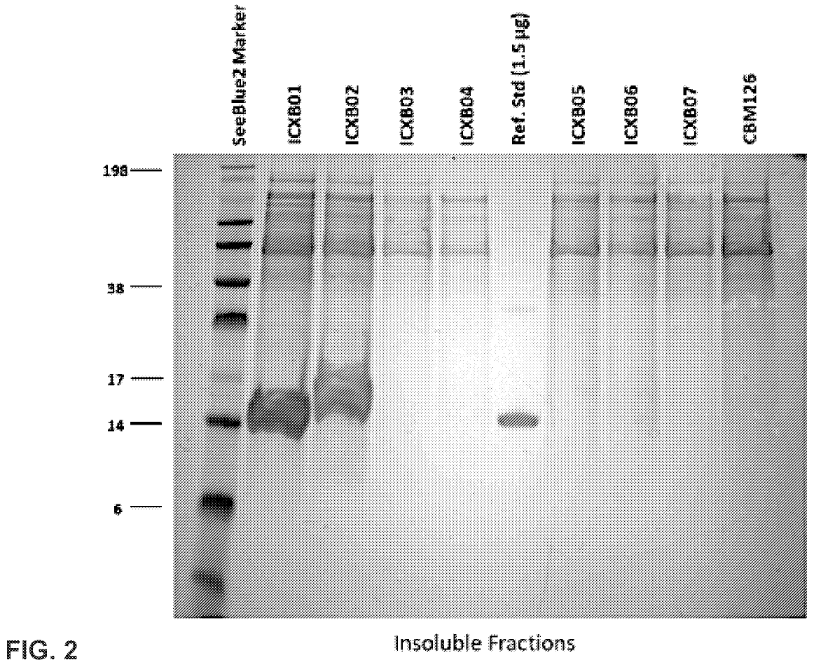

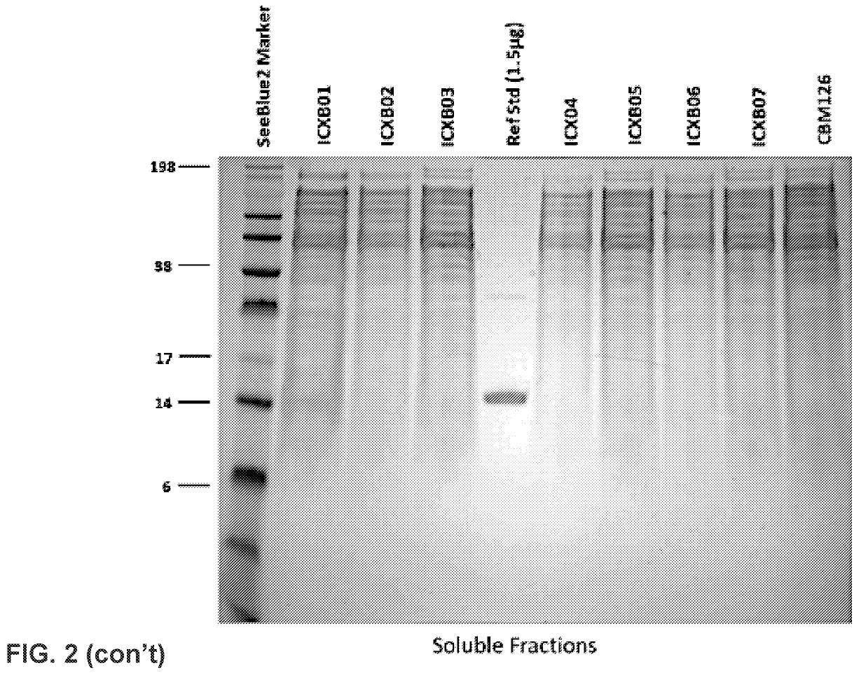

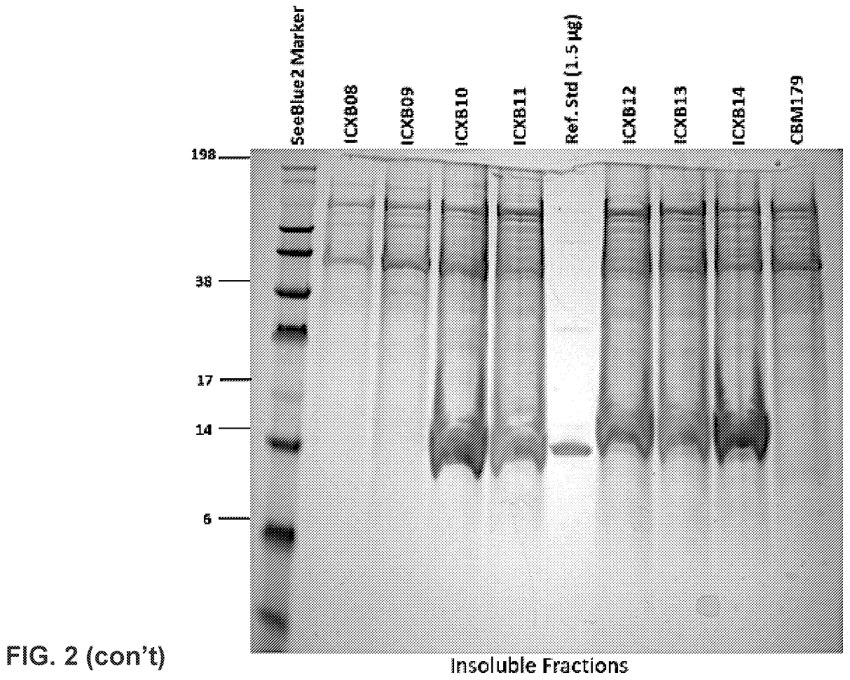

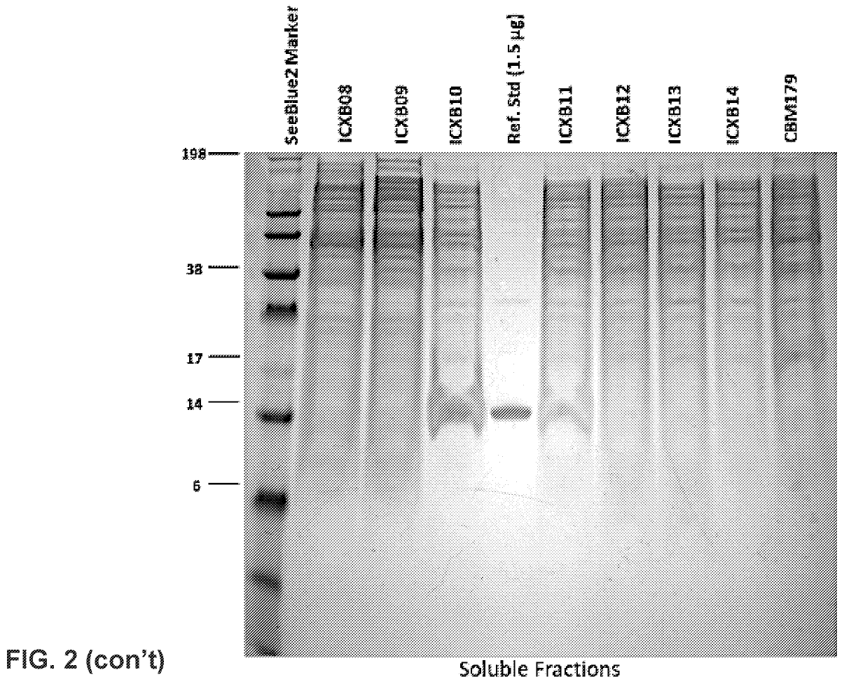

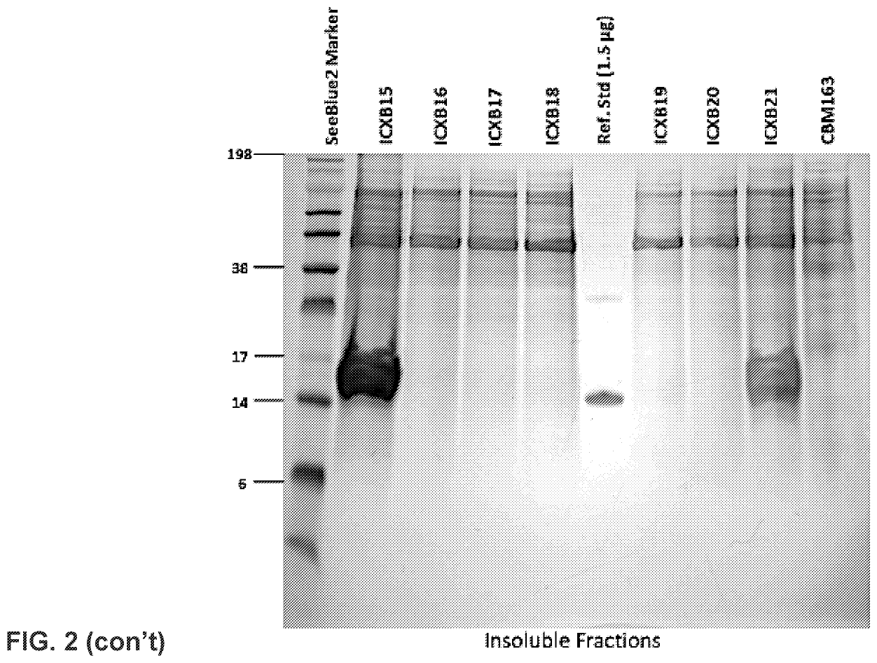

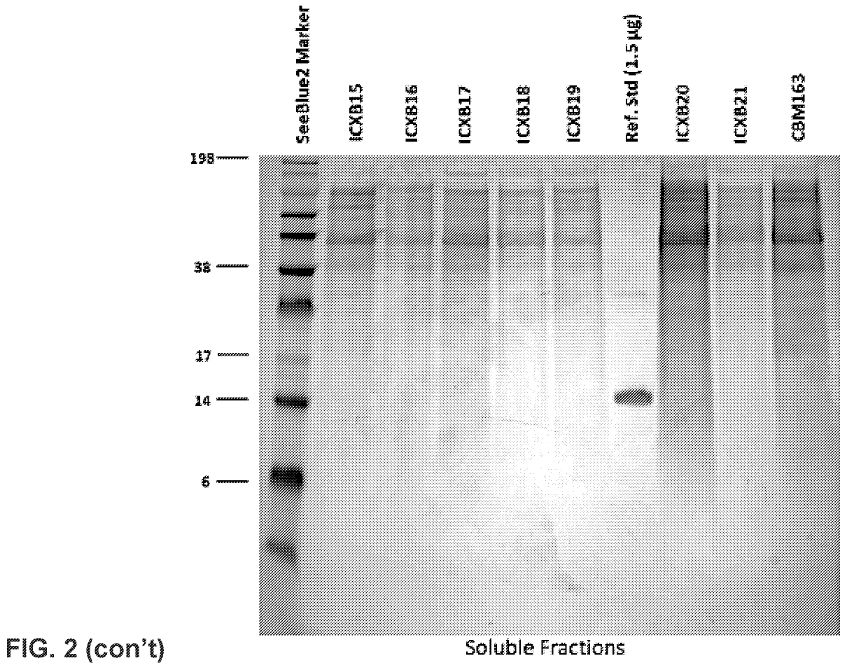

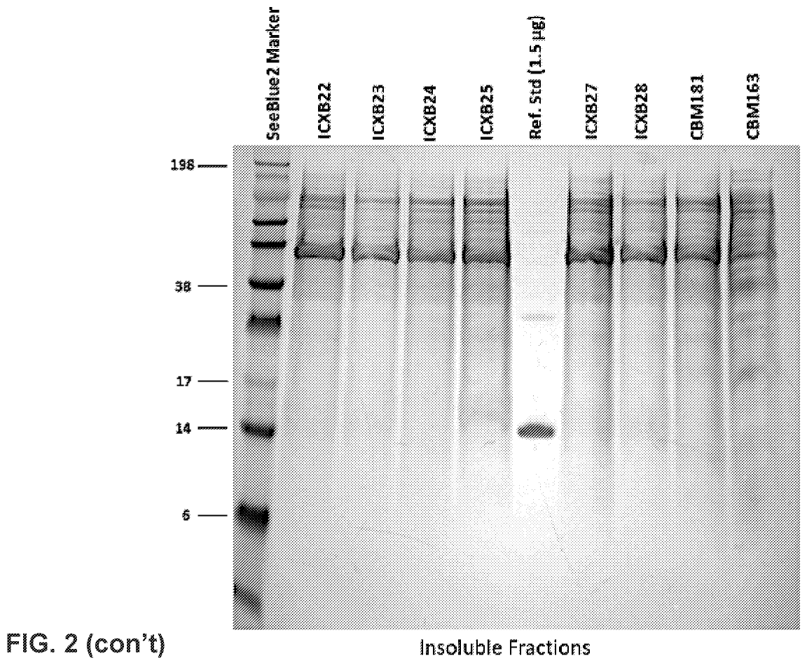

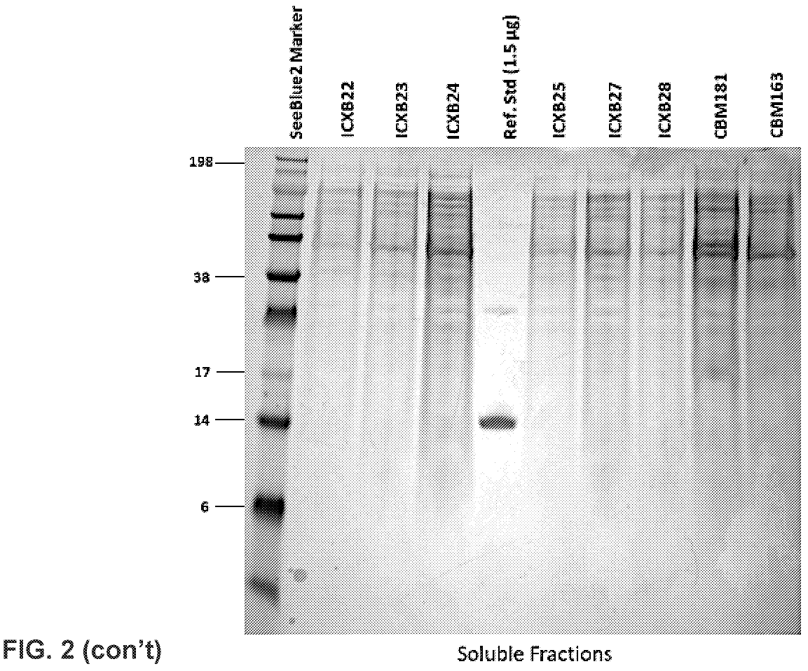

All plasmids were successfully transformed into the designated hosts. The product band was observed by SDS-PAGE mostly in the insoluble fractions, but was also present in the soluble fraction of ICXB10. The strains that produced putative products that migrated as a single band and at the approximate size of the reference standard on SDS-PAGE were chosen for further processing. The major band from ICXB01, ICXB10, ICXB11, and ICXB15 from the insoluble fractions were cut from PVDF electrotransfers stained with Ponceau S. The N-terminus of each was analyzed by Edman degradation and found to be correct for ICXB01, 1CXB10, and ICXB11. The PhoA leader peptide was still present in ICXB15 so it was not used for future work. All SDS-PAGE gels from the primary screen are presented in FIG. 2. There were no visible signs of poor growth or genetic instability in any strains. Growth and expression results are summarized in Table 5.

TABLE 5

| | | Insoluble | Soluble |
|---|---|---|---|
| Flask | Strain | expressionb | Expressionb |
| 1 | BL21 | — | — |
| 2 | ICXB01 | 2 | 2 |
| 3 | ICXB01 | 2 | 2 |
| 4 | ICXB02 | 2* | — |
| 5 | ICXB02 | 2* | — |
| 6 | CBM048 | — | — |
| 7 | ICXB10 | 3 | 2 |
| 8 | ICXB10 | 3 | 2 |
| 9 | ICXB11 | 3 | 2 |
| 10 | ICXB11 | 3 | 2 |
| 11 | ICXB12 | 3* | — |
| 12 | ICXB12 | 3* | — |
| 13 | ICXB13 | 2 (doublet) | — |
| 14 | ICXB13 | 2 (doublet) | — |
| 15 | ICXB14 | 3* | — |
| 16 | ICXB14 | 3* | ½ |
| 17 | ICXB15 | 3* | ½ |
| 18 | ICXB15 | 3* | — |

^{^{b}}Numbers given for expression are arbitrary values based on the brightness of the product bands and gel loading on SDS-PAGE

Based on the growth curves, expression, and N-terminal sequence data (Table 6) the strains ICXB01, ICXB10, and ICXB11 were chosen for fermentation screening.

TABLE 6
N-terminal Sequence Data Summary: Secondary Screen Cultures

| | ICXB01 | ICXB10 | ICXB11 | ICXB15 |
|---|---|---|---|---|
| | Insoluble | Insoluble | Insoluble | Insoluble |
| Cycle 1 residue | M/A/G | M/N | M/N | M/G |
| Cycle 2 residue | N | N/Q | N/(Q) | K |
| Cycle 3 residue | Q | Q/E/(H) | Q/(E) | Q |
| Cycle 4 residuea | H | H/S | E/S/H | S |
| Cycle 5 residue | S/(H) | S/H | S/(H) | T |
| Signal Strength | Strong | Strong | — | Strong |
| Confidence | Moderate | Moderate | — | High |
| Major sequencea | MNQHS | MNQHS | MNQHS | MKQST |
| Minor Sequence | | NQHSH | NQHSH | — |
| Peptide Sequence ID | COMPB-01 | COMPB-01 | COMPB-01 | PhoA |

Four 5 L fermenters were used to evaluate the expression of three strains (ICXB01, ICXB10 and ICXB11). See Table 7 for the experimental design. Online profiles indicate there were no significant excursions occurred in pH, temperature, or gas flow. The feed delivery was successfully initiated at the DO spike in each fermenter, and a reasonably stable DO profile was maintained throughout the runs. Periodic spikes in DO correspond to antifoam additions. There was a downshift in metabolism in both Fermenters A and B, which indicates the induction period was a bit long. Harvest criteria and induction kinetics are parameters that are optimized during process development. A summary of fermenter results is given in Table 8.

TABLE 7
Fermentation Screen Design

| Fermenter | Strain | Process | Induction | Comment |
|---|---|---|---|---|
| A | ICXB01 | T7 | 1 mM IPTG | N/A |
| B | ICX0B1 | T7 | 1 mM IPTG | Shift to 25° C. at induction |
| C | ICXB10 | phoA | Autoinduced | N/A |
| D | ICXB11 | phoA | Autoinduced | N/A |

TABLE 8
Fermentation Data Summary

| Parameter | Fermentor A | Fermentor B | Fermentor C | Fermentor D |
|---|---|---|---|---|
| Strain Inoculated | ICXB01 | ICXB01 | ICXB10 | ICXB11 |
| Temperature (° C.) during induction | 30 | 25 | 30 | 30 |
| OD₆₀₀ of Inoculum at Seed | 5.43 | 5.43 | 5.30 | 5.36 |
| Total OD Units Inoculated | 190 | 190 | 143 | 145 |
| EFT at DO Spike (hr) | 9.5 | 9.5 | 9.5 | 11.0 |
| OD₆₀₀ at DO Spike | 19.4 | 20.4 | 18.5 | 17.5 |
| WCW at DO Spike (g/L) | 32.5 | 35.5 | 46.0 | 41.0 |
| EFT at Induction (hr) | 15 | 15 | — | — |
| OD₆₀₀ at Induction (hr) | 46.9 | 46.7 | 71 | 75 |
| 50% Glucose Feed Used (g) | 1005 | 973 | 1413 | 1379 |
| Acid Used (g) | 17 | 38 | 423 | 326 |
| 30% NH₄OH Base Used (g) | 70.0 | 86.0 | 239.0 | 182.0 |
| Total EFT (hr) | 27 | 27 | 48 | 48 |
| Harvest WCW (g/L) | 178 | 190 | 194 | 177 |
| Final Harvest pellet (g) | 678 | 698 | 740 | 661 |
| Harvest WCV (%) | 17.78 | 18.91 | 19.4 | 17.7 |

In conclusion, three E. coli strains, ICXB01, ICXB10 and ICXB11, produced reasonably high levels of insoluble compB-01 in the cytoplasm in small scale cultures. ICXB10 and ICXB11 produced significant amounts of material in an initial, unoptimized 5 L fed-batch fermentation. Expression of compB-01 in the cytoplasm in ICXB10 and ICXB11 strains is controlled by the phoA promoter, which induces expression as inorganic phosphate levels in the medium are depleted. ICXB10 and ICXB11 produced inclusion bodies of compB-01 with the correct N-terminus that can be processed into assembly-competent product.

### References

The following references are incorporated by reference in there entireties.

Baneyx, F. and G. Georgiou. 1991. Construction and Characterization of Escherichia coli Strains Deficient in Multiple Secreted Proteases: Protease III Degrades High-Molecular-Weight Substrates In Vivo. J. Bacteriol. 173(8):2696- 2703.

Demerec, M., Adelberg, E. A., Clark, A. J., and P. E. Hartman. 1968. A Proposal for a Uniform Nomenclature in Bacterial Genetics. Genetics. 54(1):61-76.

Inoue, H., Nojima, H., and H. Okayama. 1990. High efficiency transformation of Escherichia coli with plasmids. Gene. 96(1):23-8.

Kikuchi, Y., Yoda, K., Yamasaki, M., and G. Tamura. 1981. The nucleotide sequence of the promoter and the amino-terminal region of alkaline phosphatase structural gene (phoA) of Escherichia coli. Nuc. Acid. Res. 9(21): 5671-5678

Kleman, G. L., and W. R. Strohl. 1994. Acetate Metabolism by Escherichia coli in High-Cell Density Fermentation. App Env Microbiol. 60(11):3952-3958

Laird, M. W., Cope, K., Atkinson, R., Donahoe, M., Johnson, K., and M. Melick. 2004. Keratinocyte Growth Factor-2 Production in an ompT-Deficient Escherichia coli K-12 Mutant. Biotechnol. Prog. 20:44-50.

McFarland, N. C. et al. 1994. Method for Refolding Insoluble Misfolded Insulin-like Growth Factor-I into an Active Conformation. U.S. Pat. No. 5,288,931.

Muller-Hill, B., Crapo, L, and W. Gilbert. 1968. Mutants That Make More of the Lac Repressor. Proc. Nat. Acad. Sci. 59(4):1259-1264.

Simmons, L. C., Reilly, D., Klimowski, L., Raju, T. S., Meng, G., Sims, P., Hong, K., Shields, R. L., Damico, L. A. Rancatore, P., and D. G. Yansura. 2002. Expression of Full-length Immunoglobins in Escherichia coli: Rapid and Efficient Production of Aglycosylated Antibodies. J. Imm. Meth. 263:133-147.

Simmons, L. C., and D. G. Yansura. 1996. Translational Level is a Critical Factor for the Secretion of Heterologous Proteins in Escherichia coli. Nat Biotech. 14:629-634.

Strauch, K. L., Johnson, K., and J. Beckwith. 1989. Characterization of degP, a Gene Required for Proteolysis in the Cell envelope and Essential for Growth of Escherichia coli at High Temperature. J. Bacteriol. 171(5):2689-2696.

Studier, F. W., and B. A. Moffatt. 1986. Use of bacteriophage T7 RNA polymerase to direct selective high-level expression of cloned genes. J Mol Biol. 189(1):113-30.

Wechselberger, P., Sagmeister, P., Engelking, H., Schmidt, T., Wenger, J., and

C. Herwig. 2012. Efficient feeding profile optimization for recombinant protein production using physiological information. Bioprocess Biosyst Eng 35:1637-1649.

### Example 2: Inclusion Body Extraction

This example demonstrates recovery of compB-01 from the inclusion bodies produced by the methods disclosed in Example 1. Specifically, following harvest of E. coli production cultures, a cell paste is resuspended in a homogenization solution (8.4 mM Sodium Phosphate, 1.5 mM Potassium Phosphate, 2.7 mM Potassium Chloride, 13 7mM Sodium Chloride, pH 7. 4). Following homogenization, the inclusion bodies are washed in four solutions as shown in Table 9.

TABLE 9
Inclusion Body Wash Solutions

| Wash | Solution |
|---|---|
| IB Wash 1 | 8.4 mM Sodium Phosphate, 1.5 mM Potassium Phosphate, 2.7 mM |
| | Potassium Chloride, 137 mM Sodium Chloride, 0.1% Triton X-100, pH 7.4 |
| IB Wash 2 | 8.4 mM Sodium Phosphate, 1.5 mM Potassium Phosphate, 2.7 mM |
| | Potassium Chloride, 137 mM Sodium Chloride, 0.1% Triton X-100, pH 7.4 |
| IB Wash 3 | 40 mM Sodium Phosphate, 1M Sodium Chloride, pH 7.4 |
| IB Wash 4 | 8.4 mM Sodium Phosphate, 1.5 mM Potassium Phosphate, 2.7 mM |
| | Potassium Chloride, 137 mM Sodium Chloride, 0.1% Triton X-100, pH 7.4 |

100 g of washed inclusion body (WIB) was resuspended with polytron in 25 mL/g WIB of Triton washing buffer (0.1% Triton X-100 in 1×PBS, pH 7.4), stirred at room temperature for 2 hours. The sample was centrifuged at 11,000 g for 30 min at 4° C. and the supernatant discarded. Pelleted WIB was then resuspended with polytron in 50 mL/g WIB of extraction buffer (20 mM Sodium Phosphate, 150 mM Sodium Chloride, 0.5 M urea, 0.75% CHAPS, pH 7.4). The extractions were incubated with mixing at ambient temperature for 2 hrs. The samples were centrifuged for 30 min at 11,000 g at 4° C. and the supernatant was filtered using a 0.22 μm filter.

### DEAE Sepharose

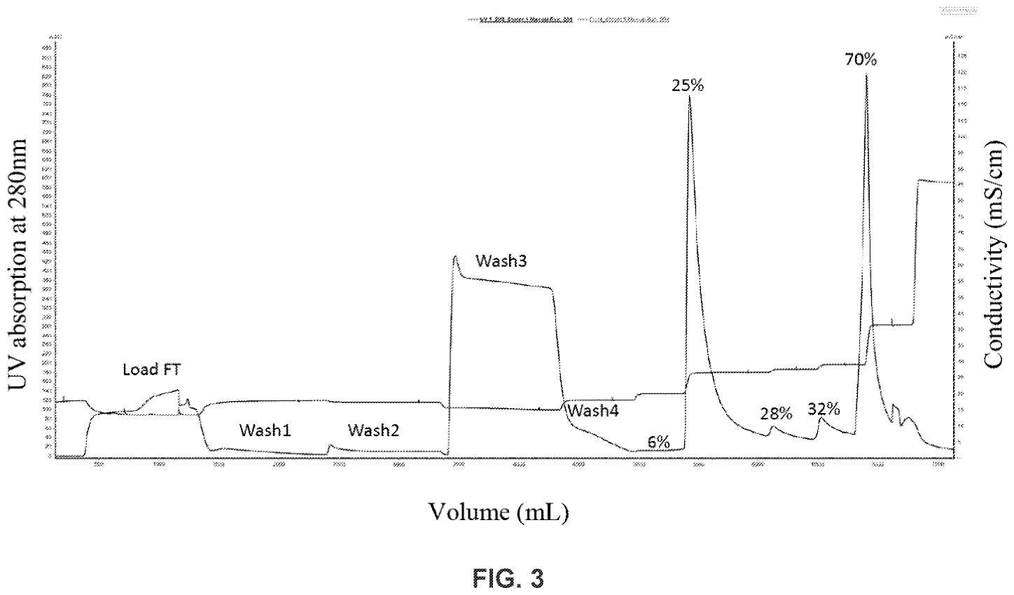

Filtered supernatant was loaded onto a DEAE Sepharose FF column. Anion exchange chromatography was performed using a step gradient according to the following process parameters. A representative chromatogram is shown in FIG. 3.

Resin DEAE Sepharose Fast Flow
Column dimensions 5.0 cm (d) × 10.2 cm (h)
Column cross sectional area 19.6 cm²
Column volume 200 mL
Load Density 15 mg/mL resin based on A280 value

| | | | Velocity | | |
|---|---|---|---|---|---|
| Process Step | Buffer | CV | (cm/hr) | (mL/min) | (min/CV) |
| Sanitization | 0.5 NaOH (≥30 min) | 3 | 122 | 40 | 5 |
| Flush | Water | 2 | 122 | 40 | 5 |
| Flush 2 | 100 mM Tris, 1.5M NaCl, pH 8.0 | 3 | 122 | 40 | 5 |
| Recharge | 20 mM NaPO₄, 1M NaCl, pH 7.4 | 3 | 122 | 40 | 5 |
| Equilibration | 20 mM NaPO₄, 150 mM NaCl, pH | 5 | 122 | 40 | 5 |
| | 7.4 | | | | |
| Load | Sample | TBD | 122 | 40 | 5 |
| Wash 1 | 20 mM NaPO₄, 150 mM NaCl, pH | 5 | 122 | 40 | 5 |
| | 7.4 | | | | |
| Wash 2 | 20 mM NaPO₄,150 mM NaCl, 0.75% | 5 | 122 | 40 | 5 |
| | CHAPS, pH 7.4 | | | | |
| Wash 3 | 20 mM NaPO₄, 150 mM NaCl, 0.1% | 5 | 122 | 40 | 5 |
| | Triton X100 pH 7.4 | | | | |
| Wash 4 | 20 mM NaPO₄, 150 mM NaCl, pH | 5 | 122 | 40 | 5 |
| | 7.4 | | | | |
| Step Gradient Elution | A) 20 mM NaPO₄, 150 mM NaCl, | 7 | 122 | 40 | 5 |
| 68, 258, 28% 32% | pH 7.4 | | | | |
| and 70% Buffer B | B) 20 mM NaPO₄, 500 mM NaCl, | | | | |
| | pH 7.4 | | | | |
| Strip | 20 mM NaPO₄, 1M NaCl, pH 7.4 | 3 | 122 | 40 | 5 |
| Sanitization | 0.5N NaOH (≤20 min) | 3 | 122 | 40 | 5 |
| Flush | Water | 2 | 122 | 40 | 5 |
| Storage | 20% Ethanol | 3 | 122 | 40 | 5 |

The elution at 25% (F1) from peak ascending for 2 column volumes (CV) was collected for the next step in purification.

### CHT Chromatography

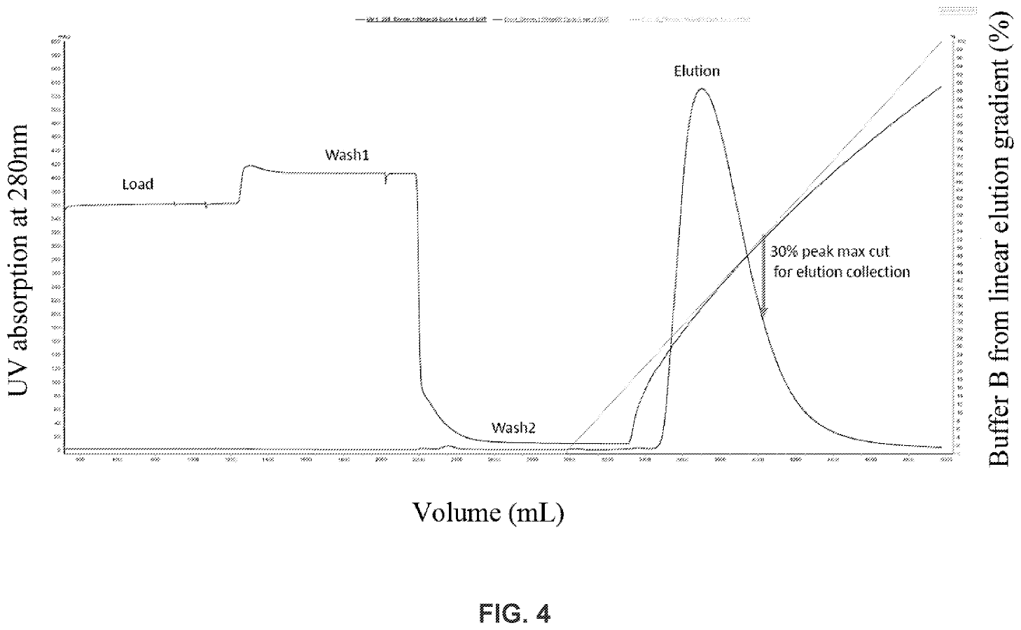

The collected sample was loaded onto CHT Ceramic Hydroxyapatite Type I media column. Mixed-mode chromatography was performed according to the following process parameters. A representative chromatogram is shown in FIG. 4.

Resin CHT Type 1, 40 μm
Column dimensions 5.0 cm (d) × 9.5 cm (h)
Column cross sectional area 19.6 cm²
Column volume 200 mL
Loading Capacity 10 mg/mL resin based on A280 value
Process Step Buffer

| | | | Velocity | | |
|---|---|---|---|---|---|
| Process Step | Buffer | CV | (cm/hr) | (mL/min) | (min/CV) |
| Sanitization | 0.5 N NaOH (≤60 min) | 3 | 122 | 40 | 5 |
| Flush | UPW | 2 | 122 | 40 | 5 |
| Regeneration | 500 mM NaPO₄, pH 7.4 | 3 | 122 | 40 | 5 |
| Equilibration | 5 mM NaPO₄, 200 mM | 5 | 122 | 40 | 5 |
| | NaCl, pH 7.4 | | | | |
| Load | compB Fraction from | TBD | 122 | 40 | 5 |
| | DEAE elution at 25% | | | | |
| Wash1 | 5 mM NaPO₄, 300 mM | 5 | 122 | 40 | 5 |
| | NaCl, pH 7.4 | | | | |
| Wash2 | 5 mM NaPO₄, PH 7.4 | 5 | 122 | 40 | 5 |
| Elution | 5 mM to 500 mM NaPO₄ | 10 | 122 | 40 | 5 |
| | in 10 CV, pH 7.4 | | | | |
| Sanitization | 1N NaOH (5 CV) | 5 | 122 | 40 | 5 |
| Flush | UPW | 2 | 122 | 40 | 5 |
| Storage | 0.1N NaOH | 3 | 122 | 40 | 5 |

Eluate collection was started from elution peak ascending to 30% of peak maximum on the descending side of the peak.

### Ultrafiltration/Diafiltration

Ultrafiltration/Diafiltration (UFDF) formulation was carried out on CHT eluate (2958.4 mg). Millipore Pellicon 2 Mini Cassette, Biomax 10 kDa (Cat #: P2B010A01, 0.1 m2) was used. CHT eluate was concentrated to about 4 mg/mL (sample volume ˜700 mL) and diafiltered against formulation buffer (20 mM Tris, 250 mM NaCl, pH 7.4) for 8 DV (5600 mL) using a feed pump flow rate of 500 mL/min. The UFDF product was 0.22 μm filtered, and the concentration was adjusted to 2.55 mg/mL, the total volume was 1051 mL, final protein yield=2680 mg. Aliquots were stored at −80° C.

Endotoxin testing demonstrated that the product met desired criteria, as shown in the following table.

| | | | Endotoxin, | |
|---|---|---|---|---|
| | | Concentration | by volume | Endotoxin, |
| Sample Description | Lot Number | (mg/mL) | (EU/mL) | (EU/mg) |
| DEAE Load | 16-003 | 3.50 | 1293258.662 | 369,502 |
| DEAE Seph FF 25% | 16-004 | 1.51 | 131.052 | 87 |
| Elution | | | | |
| CHT Eluate | 16-005 | 1.80 | 34.382 | 19 |
| Final Formulation | A | 2.55 | 26.771 | 10 |

In conclusion, about 1.35 g of final Comp-B product can be obtained from 1 L of cell broth. The final formulation was changed to 20 mM Tris, 250 mM NaCl, pH 7.4, based on the formulation results. CHT column elution collection cut off was determined to be at 30% of peak max at the descending side of the elution peak. Step yield was 77%. Endotoxin level was reduced by 4-fold. The final UFDF reduces endotoxin level. Overall process yields are summarized in the following table.

Yield Summary

| | |
|---|---|
| Temperature (° C.) | 30 |
| Phosphate (mM) | 16.4 |
| Harvest (g/L) | 190 (B), 202 (C) |
| Amount of paste processed (g) | 1250 |
| Amount of IB (g) | 366.5 |
| Amount of IB processed for Purification (g) | 100 |
| DEAE (25% Elution) (g) | 3.86 |
| CHT Run Eluate (30% Peak Max Cut) (g) | 2.96 |
| UF/DF and Final Filtration (g) | 2.68 |
| Final product (g)/ L Fermentation broth | 1.35 |
| Residual DNA (pg/mg) | 9.5 |
| Endotoxin (EU/mg) | 10 |

### Example 3: Inclusion Body Extraction

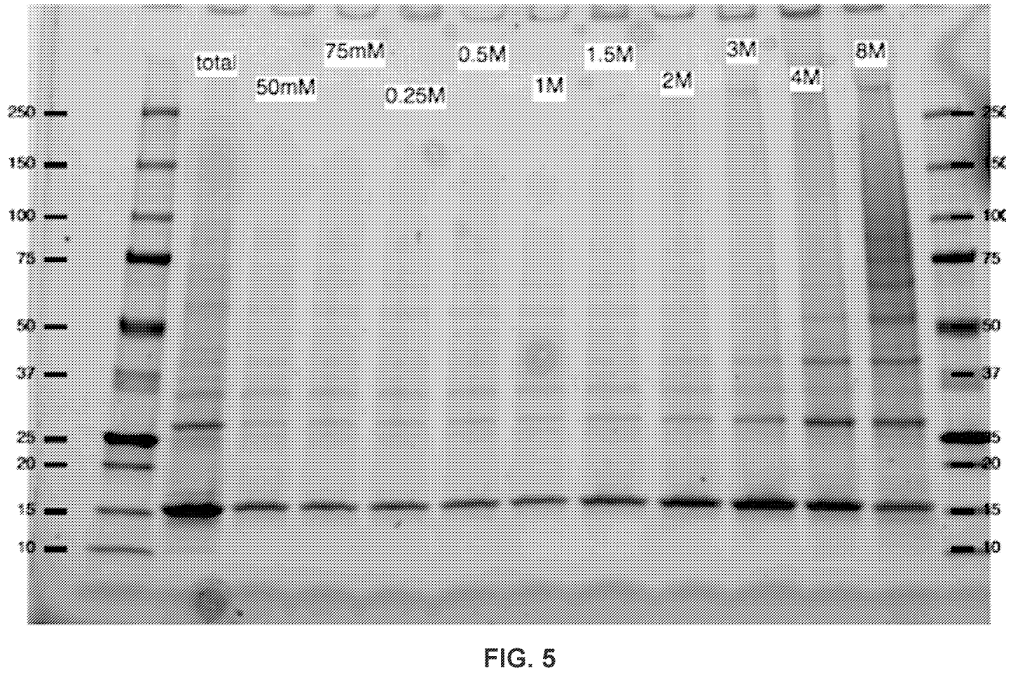

This example demonstrates further refined of the inclusion body extraction method described in Example 2. Several options for insoluble pellet washes were examined, including alternative detergents. I53-50B cell pellets were resuspended in PBS at 10 mL/g of wet cell weight and homogenized for 1 minute using an IKA Ultra-Turrax T25 homogenizer at 4000 rpm. The resuspended cells were lysed using a Microfluidics M110P microfluidizer, three discrete passes at 18000psi, at 2-8° C. The lysate was collected in a clean container, portioned into 50 mL Falcon tubes, and clarified by centrifugation at 24000 g for 30 minutes at 4° C. The soluble fraction was removed to a new container. An initial experiment determined at which concentration of urea the compB protein became solubilized, beginning at 50 mM and increasing to 8M, using aliquots of lysate and equal volumes of PBS with increasing urea molarity for 2 hours. Results are shown in FIG. 5. The the compB protein began to extract at 50 mM urea, so low concentrations of chaotrope were not pursued as a wash step.

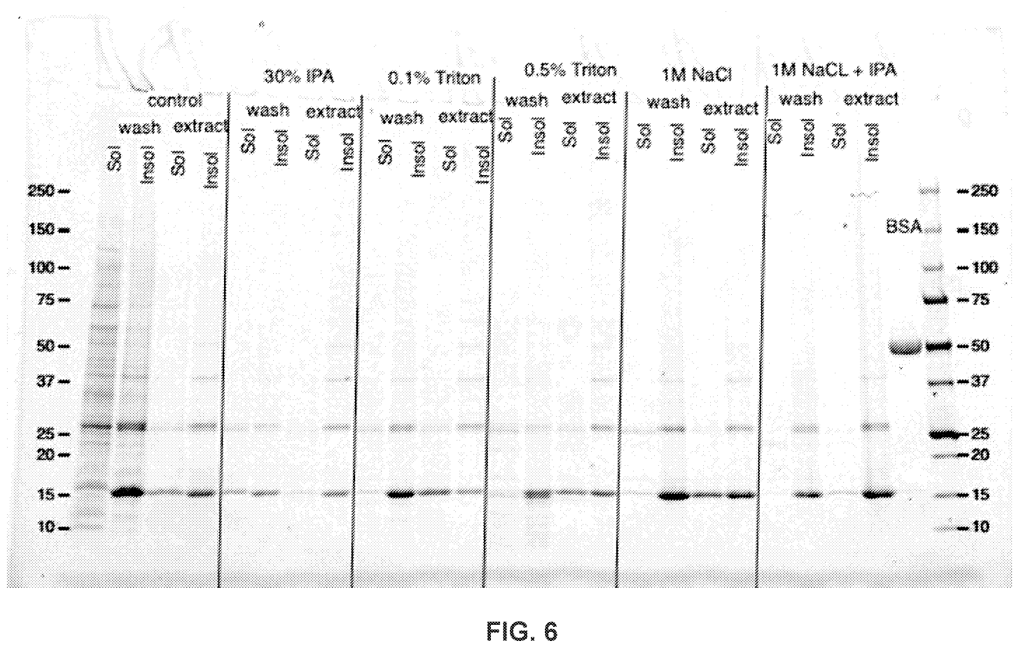

Subsequent experiments examined an alternative detergent, Triton X-100, high salt, and 30% isopropanol, with purity by SDS-PAGE and endotoxin clearance as the primary readouts. Results are shown in FIG. 6. The control sample did not have a wash step but proceeded directly to extraction using PBS 2M urea 0.75%(w/v) Chaps pH 7.4. Test samples were washed with one of the following wash buffers, all in a PBS background:
a. 30% isopropanol,
b. 0.1% Triton X-100,
c. 0.5% Triton X-100,
d. 1M NaCl, and
e. 1M NaCl 30% isopropanol.

The isopropanol appeared to negatively impact solubility, with less compB in the extracted fraction. The 1M NaCl appears to remove host cell proteins, but does not impact endotoxin clearance. The 0.1% Triton X-100 demonstrated the best clearance factor. Moving forward, two wash steps will be performed, with Wash 1 being PBS 0.1% Triton X100 and Wash 2 is 20 mM NaPO4 1M NaCl. Results are summarized in the table that follows.

| Wash | EU/mL | % difference |
|---|---|---|
| control | >50,000 | from control |
| 0.1% Triton | 24,242 | 69.2 |
| 0.5% Triton | 32,397 | 42.7 |
| 1M NaCl | >50,000 | 0 |

Subsequently, fermentation was scaled up to four 5 liter fermenters at 30° C. 1.25 kg of cell paste from Fermenter B (696.1 g) and Fermenter C (553.9 g) was resuspended, lysed and washed to recover 366.5 g of washed TB, which corresponds to 29% of initial weight. Inclusion bodies were resuspended in 25 mL/g of 0.1% Triton X-100 in phosphate buffered saline (PBS), pH 7.4 and stirred at room temperature for two hours. Inclusion bodies were recovered by centrifugation at 11,000 g. Washed inclusion bodies were then extracted with 20 mM sodium phosphate, 150 mM, 0.5 M urea, 0.75% CHAPS, pH 7.4. The supernatant was cleared by centrifugation at 11,000 g and subjected to further purification.

### DEAE Sepharose FF Chromatography

Combinations of Triton X-100 and CHAPS were tested. A 10 cm bed height DEAE column was equilibrated with 20 mM NaPO4 150 mM NaCl pH 7.4, and washed with different column-wash buffers:
a. 20 mM NaPO₄ 150mM NaCl 0.1% Triton X-100 pH 7.4,
b. 20 mM NaPO₄ 150mM NaCl 0.75% Chaps pH 7.4, or
c. both in series.

Elution was accomplished with 5CV of 20 mM NaPO₄ 500 mM NaCl, then the column was stripped with 5CV each of 20 mM NaPO₄ 1M NaCl pH 7.4 and sanitized with 0.5M NaOH.

The purity of the elution as determined by nonreducing SDS-PAGE was comparable between the three options, but endotoxin removal was demonstrably most efficient with the combination of the two detementc

| Process | Recovery | Endotoxin (EU/mL) |
|---|---|---|
| Tris | 70% | 3999 |
| CHAPS | 60% | 1060 |
| Tris then CHAPS | 45% | 824 |

### Assembly Competence

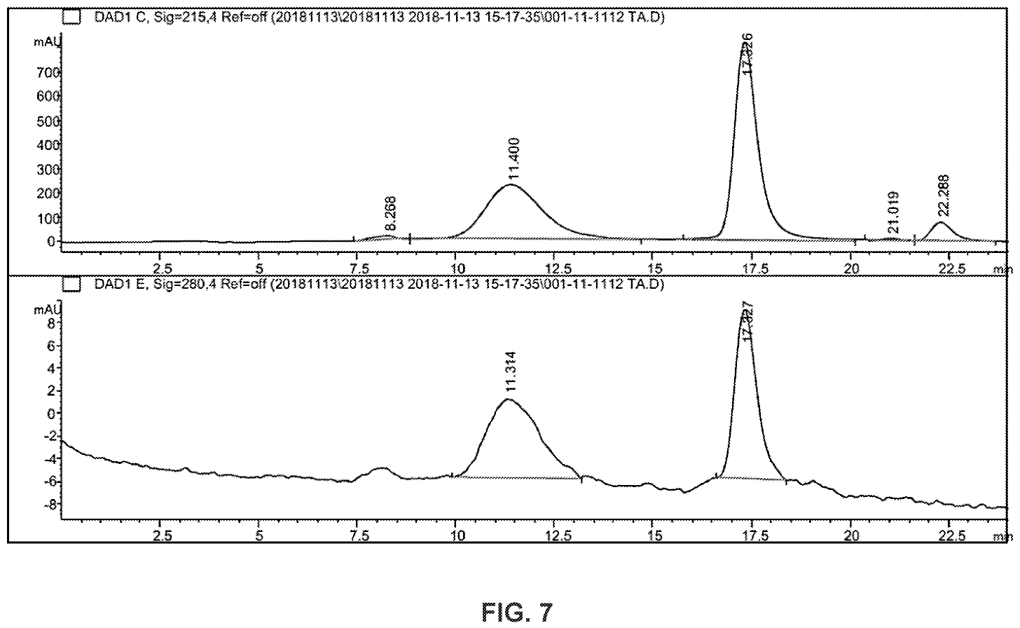

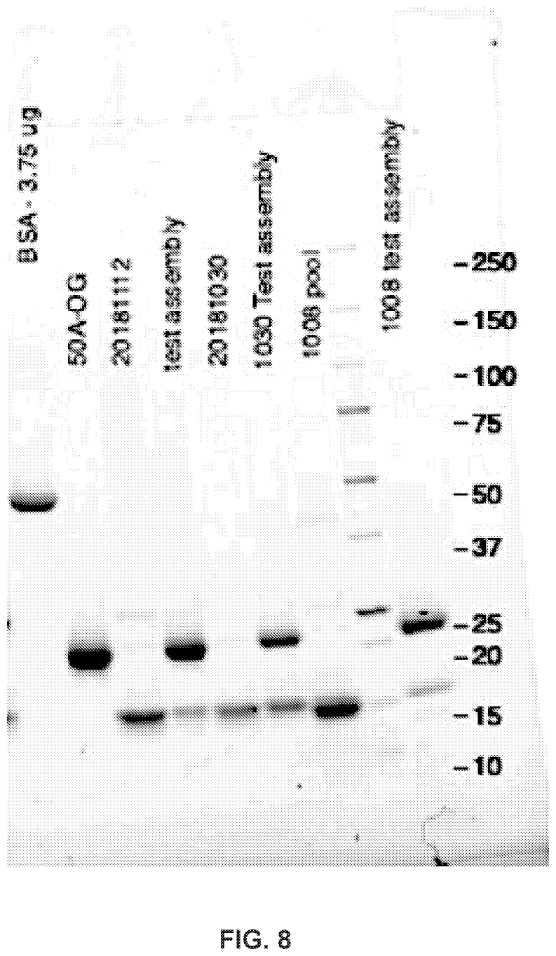

The ability of the purified I53-50B to assemble with I53-50A to the designed icosahedral architecture upon mixing in vitro was analyzed by gently mixing purified components in a 1:1 molar ratio. Mixtures were then analyzed on a Superose 6 Increase 10/300 GL gel filtration column (GE Life Sciences) using 20mM NaPO4, 150 mM NaCl pH 7.4 as mobile phase. Representative chromatograms are shown in FIG. 7. SDS-PAGE analysis of assembly mixtures is shown in FIG. 8. Monodisperse nanoparticle has a retention time of ˜11.3 minutes, I53-50A alone at 17.2 minutes.

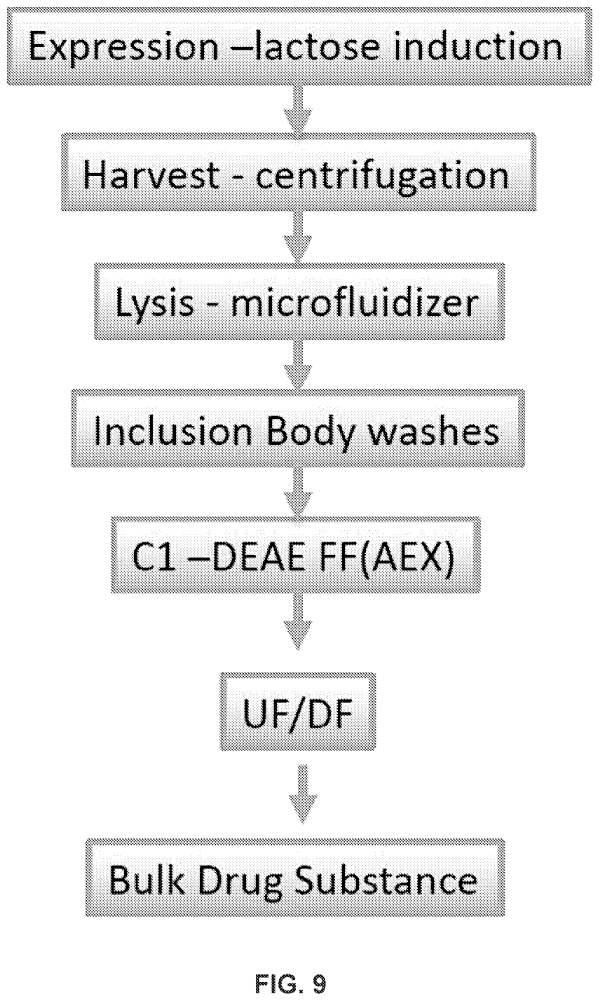

In conclusion, the purified I53-50B is both assembly competent and not aggregated. The purification process flow shown in FIG. 9 produced pure, assembly-competent I53-50B.

### Example 4: Scale-up Manufacturing and Purification of Component B

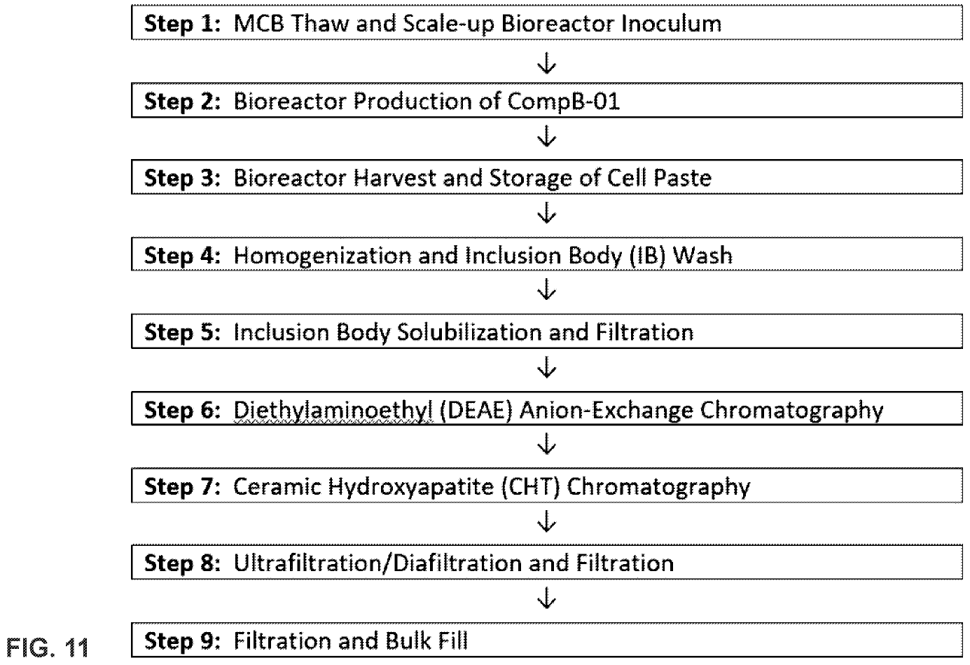

For manufacturing of compB-01 (I53-50B sequence), scalable processes were developed for unit operations including bioreactor production and harvest, extraction of soluble pentamer from the insoluble fraction and downstream processing using a two-step chromatography process followed by UF/DF for final formulation. An overview flow diagram of the compB-01 drug substance intermediate manufacturing process is provided in FIG. 11.

For initial proof-of-concept at small-scale, the compB-01 manufacturing process was executed at the 2×2.5 L bioreactor scale, resulting in 2.6 g of final drug substance intermediate (compB Demo Lot A; 0618-60). Scale-up, current good manufacturing protocol (cGMP) manufacturing of compB-01 was executed at the 200 L bioreactor scale with approximately 10% of the resulting bioreactor harvest cell paste used for subsequent downstream processing, resulting in 19.0 g of final drug substance intermediate (compB-01 GMP Lot B; 20-4076).

A comparison of the manufacturing scale for compB-01 Demo Lot A and compB-01 GMP Lot B is provided in Table 10. Successful scale-up of the compB-01 manufacturing process is highlighted by both the levels of cell paste recovered following bioreactor harvest, and a 7-fold increase in final drug substance intermediate through downstream processing using a 10-fold higher level of inclusion bodies for the GMP process. Successful scale-up of the compB-01 manufacturing process is also demonstrated by the analytical characterization data provided in Table 11 where both lots of material were demonstrated to have comparable product quality attributes.

TABLE 10
Scalable Manufacturing of CompB-01 Drug Substance Intermediate

| Stage | Demo (Lot A) | GMP (Lot B) | Scale Factor |
|---|---|---|---|
| Thaw and | 2 × 200 mL in 1 L flask | 4 × 400 mL in 2 L flask | 4-fold |
| Expansion | | | |
| Production | 2 × 2.5 L working | 200 L working volume | 40-fold |
| Bioreactor | volume STR | STR | |
| Bioreactor | 1.25 kg cell paste | 29.3 kg cell paste | 23-fold |
| Harvest | harvested | harvested | |
| IB Isolation | 1.25 kg starting cell | 3.8 kg starting cell paste¹ | 3-fold |
| | paste | | |
| IB resuspension | 0.1 kg starting washed | 1.0 kg starting washed | 10-fold |
| | IB¹ | IB¹ | |
| DEAE | 14.3 g of solubilized IB | 105 g of solubilized IB | 7-fold |
| Chromatography | processed using 4 | processed using 1 cycle | |
| | cycles over 0.2 L resin | over 12.4 L resin | |
| CHT | 3.9 g of DEAE elution | 25.7 g of DEAE elution | 7-fold |
| Chromatography | processed using 3 | processed using 1 cycle | |
| | cycles over 200 mL | over 5.4 L resin | |
| | resin | | |
| UF/DF | 3.0 L CHT elution | 12.7 L CHT elution | 4-fold |
| | processed using 0.1 m² | processed using 0.5 m² | |
| | membrane | membrane | |
| Bulk Fill | 2.6 g of compB-01 drug | 19.0 g of compB-01 drug | 7-fold |
| | substance intermediate | substance intermediate | |

¹Process scale deliberately adjusted to accommodate downstream unit operations.

TABLE 11
Analytical Characterization of CompB-01 Drug Substance Intermediate.

| | Results | |
|---|---|---|
| Attribute | B (GMP) | A (Non-GMP) |
| pH | 7.2 | 7.41 |
| Identification by SE-HPLC | Conforms | Conforms |
| Identification by CE-SDS | Conforms | Conforms |
| Protein content (mg/mL) | 2.6 | 2.9 |
| Purity by CE-SDS (%) | | |
| Reduced | >99 | 98.0 |
| Non-reduced | 99 | 96.6 |
| Host cell DNA (ng/mg) | <0.001 | 9.462 × 10⁻³ |
| Host cell protein (ng/mg) | 1 | 9 |
| Assembly competence | 1.99 | 0.8 |
| Endotoxin (EU/mg) | 1.186 | 10 |
| Bioburden (CFU/mL) | | 0 |
| TAMC | 0 | |
| TYMC | 0 | |

CE-SDS = capillary electrophoresis sodium dodecyl sulfate;
CFU = colony forming unit;
DNA = deoxyribonucleic acid;
EU = endotoxin unit;
SE-HPLC = size exclusion high-performance liquid chromatography

### Alternate CompB Production

For current GMP manufacturing, bompB-01 (I53-50B sequence) is produced in E. coli and extracted as a soluble pentamer for downstream processing.

For current GMP manufacturing of I53-50B bompB-01, the production strain IXCB10 DCB (Lot 191100308) was generated by cloning of the I53-50B sequence into the pCTY13 vector backbone for transformation of E. coli strain CBM179. The pCYT13 vector using a phoA promoter that drives compB-01 expression following phosphate depletion in the media and results in soluble compB-01 pentamer that can be extracted from the insoluble fraction under relatively mild conditions.

For evaluation of the alternate molecule using the sample manufacturing platform, the I53-dn5A DNA sequence was also cloned into the pCTY13 vector backbone for transformation of E. coli strain CBM179, resulting in the expression strain IVXB30. For evaluation of I53-dn5A compB production, a 1000 mL shake flask production culture was grown using standard procedures for evaluation of I53-dn5Aexpression. Following production, soluble and insoluble fractions were isolated from cell pellets and evaluated by SDS-PAGE.

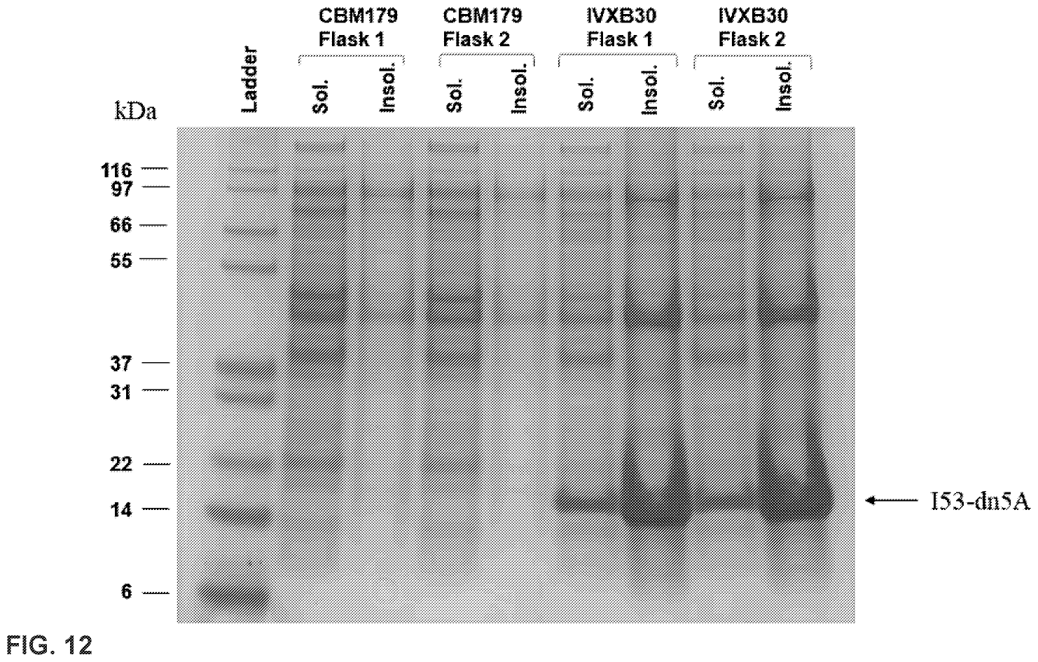

As shown in FIG. 12, significant levels of I53-dn5A were observed in the insoluble fraction from E. coli production cultures. These results are consistent with expression patterns observed for I53-50B compB-01 and support use of a common manufacturing platform for distinct soluble pentamers.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A method of making a nanostructure, comprising solubilizing a recombinant component B (compB) protein from inclusion bodies with a solubilization solution, thereby generating a product sample comprising product compB protein.

2. The method of claim 1, wherein the solubilization solution comprises urea.

3. The method of claim 1, wherein the solubilization solution is a buffered solution having a pH of 7-8.

4. The method of claim 1, wherein the solubilization solution comprises a zwitterionic surfactant.

5. The method of claim 1, wherein the solubilization solution comprises 3-[(3-cholamidopropyl)dimethylammonio]-1-propanesulfonate (CHAPS).

6. The method of claim 1, wherein the method comprises, prior to the solubilization step, washing the inclusion bodies with a wash solution comprising urea.

7. The method of claim 1, wherein the method comprises contacting the compB protein with an anion exchange resin; and eluting the compB protein from the resin using an elution solution.

8. The method of claim 7, wherein the method comprises, before the eluting step, washing the anion exchange resin with a column-wash solution, the column-wash solution comprising:
a zwitterionic surfactant, or
a nonionic surfactant.

9. The method of claim 7, wherein the elution solution comprises sodium chloride (NaCl) at a NaCl concentration of 400 mM to 600 mM.

10. The method of claim 1, wherein the method comprises purifying the compB protein with a mixed-mode resin.

11. The method of claim 1, wherein the inclusion bodies were generated in a bacterial cell comprising a polynucleotide encoding the compB protein, the polynucleotide operatively linked to a promoter.

12. The method of claim 11, wherein the bacterial cell is cultured at less than about 33° C.

13. The method of claim 11, wherein the bacterial cell is an E. coli cell.

14. The method of claim 13, wherein the bacterial cell is a B-strain E. coli cell.

15. The method of claim 13, wherein the bacterial cell is a K12-strain E. coli cell.

16. The method of claim 11, wherein the promoter is a PhoA promoter.

17. The method of claim 11, wherein the promoter is a promoter other than a T7 promoter.

18. The method of claim 11, wherein the method comprises lysing the bacterial cell in a lysis solution, wherein the lysis solution is substantially free of agents that promote solubility of inclusion bodies; and recovering the inclusion bodies.

19. The method of claim 18, wherein the lysis solution is substantially free of detergents.

20. The method of claim 1, wherein the product compB protein has at least 50% solubility.

21. The method of claim 20, wherein solubility is measured by gel filtration chromatography.

22. The method of claim 1, wherein the product compB protein has at least 80% purity calculated as weight by weight of total protein (w/w).

23. The method of claim 22, wherein purity is measured by poly-acrylamide gel electrophoresis.

24. The method of claim 1, wherein the product compB protein is at least 70% w/w assembly competent.

25. The method of claim 24, wherein percentage of assembly competent compB protein is defined as the percentage of compB protein in the product solution, weight by weight (w/w), that assembles into a protein-based Virus-Like Particle (vpVLP) when the compB protein is mixed with a solution comprising component A (compA) protein in excess.

26. The method of claim 1, wherein the product solution comprises less than 50 endotoxin units per milligram of total protein (EU/mg).

27. The method of claim 1, wherein the method does not comprise denaturing the compB protein [[and/]]or does not comprises refolding the compB protein.

28. The method of claim 1, wherein the yield of compB protein is between about 170-190 g/L wet cell weight (WCW) at harvest or about 1 g/L WCW compB protein in the inclusion bodies.

29. The method of claim 1, wherein the compB protein is a I53-50B protein.

30. The method of claim 29, wherein the I53-50B protein shares at least 95% identity to I53-50B.1 (SEQ ID NO:32), I53-50B.1NegT2 (SEQ ID NO:33), or I53-50B.4PosT1 (SEQ ID NO: 34).

31. The method of claim 29, wherein the I53-50B is any one of the proteins represented by SEQ ID NO: 40 (I53-50B genus).

32. The method of claim 29, wherein the I53-50B protein shares at least 99% identity to I53-50B.1 (SEQ ID NO:32), I53-50B.1NegT2 (SEQ ID NO:33), or I53-50B.4PosT1 (SEQ ID NO: 34).

33. The method of claim 29, wherein the I53-50B protein shares 100% identity to I53-50B.1 (SEQ ID NO:32), I53-50B.1NegT2 (SEQ ID NO:33), or I53-50B.4PosT1 (SEQ ID NO: 34).

34. The method of claim 1, wherein the compB protein is a I53_dn5A protein. 35-37. (Canceled)

38. A composition comprising compB protein produced by the method of of claim 1.

39. A composition comprising compB protein, wherein the compB protein is:
a. at least 50% soluble;
b. at least 80% pure, wherein purity is calculated as weight by weight of total protein (w/w); or
c. at least 70% w/w assembly competent.

40-43. (canceled)

44. A nanostructure, comprising a component A (compA) protein and a component B (compB) protein, wherein the compB protein is solubilized from inclusion bodies with a solubilization solution.

45-50. (canceled)

51. A method of generating an immune response in a subject in need thereof, comprising administering an effective amount of the nanostructure of claim 4 to the subject.

52-54. (canceled)
